(19) United States
(12) Patent Application Publication
(10) Pub. No.: US 2012/0045809 A1
(43) Pub. Date: Feb. 23, 2012

(54) Engineered Microorganisms for the Production of One or More Target Compounds

(75) Inventors: Thomas Buelter, Denver, CO (US); Peter Meinhold, Denver, CO (US); Christopher Smith, Englewood, CO (US); Aristos Aristidou, Highlands Ranch, CO (US); Catherine Asleson Dundon, Englewood, CO (US); Jun Urano, Aurora, CO (US)

(73) Assignee: GEVO, INC., Englewood, CO (US)

(21) Appl. No.: 13/141,648

(22) PCT Filed: Dec. 23, 2009
(86) PCT No.: PCT/US2009/069390

§ 371 Date: Oct. 14, 2011

Related U.S. Application Data
(60) Provisional application No. 61/140,611, filed on Dec. 23, 2008.
(60) Provisional application No. 61/213,918, filed on Jul. 29, 2009.
(60) Provisional application No. 61/213,919, filed on Jul. 29, 2009.

(51) Int. Cl. C12P 7/16; C12N 1/19

(52) U.S. Cl. 435/160; 435/254.2

(57) ABSTRACT

The present invention provides recombinant microorganisms comprising an isobutanol producing metabolic pathway and methods of using said recombinant microorganisms to produce isobutanol. In various aspects of the invention, the recombinant microorganisms comprise isobutanol producing metabolic pathway with one or more isobutanol pathway enzymes localized in the mitochondria. In various embodiments described herein, the recombinant microorganisms may be Crabtree-negative yeast microorganisms, microorganisms of the Saccharomyces clade, Crabtree-positive yeast microorganisms, post-WGD (whole genome duplication) yeast microorganisms, pre-WGD (whole genome duplication) yeast microorganisms, and non-fermenting yeast microorganisms.

### CROSS-REFERENCE TO RELATED APPLICATIONS

This application claims priority to U.S. Provisional Application Ser. No. 61/140,611, filed Dec. 23, 2008, U.S. Provisional Application Ser. No. 61/213,918, filed Jul. 29, 2009, and U.S. Provisional Application Ser. No. 61/213,919, filed Jul. 29, 2009, each of which are hereby incorporated by reference in their entireties for all purposes.

### TECHNICAL FIELD

The invention is generally related to metabolically engineered microorganisms and methods of their use for the production of beneficial metabolites including C3-C5 alcohols such as isobutanol.

### BACKGROUND

Biofuels have a long history ranging back to the beginning of the 20th century. As early as 1900, Rudolf Diesel demonstrated at the World Exhibition in Paris, France, an engine running on peanut oil. Soon thereafter, Henry Ford demonstrated his Model T running on ethanol derived from corn. Petroleum-derived fuels displaced biofuels in the 1930s and 1940s due to increased supply, and efficiency at a lower cost.

Market fluctuations in the 1970s coupled to the decrease in US oil production led to an increase in crude oil prices and a renewed interest in biofuels. Today, many interest groups, including policy makers, industry planners, aware citizens, and the financial community, are interested in substituting petroleum-derived fuels with biomass-derived biofuels. The leading motivations for developing biofuels are of economical, political, and environmental nature.

One is the threat of ‘peak oil’, the point at which the consumption rate of crude oil exceeds the supply rate, thus leading to significantly increased fuel cost results in an increased demand for alternative fuels. In addition, instability in the Middle East and other oil-rich regions has increased the demand for domestically produced biofuels. Also, environmental concerns relating to the possibility of carbon dioxide related climate change is an important social and ethical driving force which is starting to result in government regulations and policies such as caps on carbon dioxide emissions from automobiles, taxes on carbon dioxide emissions, and tax incentives for the use of biofuels.

Ethanol is the most abundant fermentatively produced fuel today but has several drawbacks when compared to gasoline. Butanol, in comparison, has several advantages over ethanol as a fuel: it can be made from the same feedstocks as ethanol but, unlike ethanol, it is compatible with gasoline at any ratio and can also be used as a pure fuel in existing combustion engines without modifications. Unlike ethanol, butanol does not absorb water and can thus be stored and distributed in the existing petrochemical infrastructure. Due to its higher energy content which is close to that of gasoline, the fuel economy (miles per gallon) is better than that of ethanol. Also, butanol-gasoline blends have lower vapor pressure than ethanol-gasoline blends, which is important in reducing evaporative hydrocarbon emissions.

Isobutanol has the same advantages as butanol with the additional advantage of having a higher octane number due to its branched carbon chain. Isobutanol is also useful as a commodity chemical and is also a precursor to MTBE. Isobutanol can be produced in microorganisms expressing a heterologous metabolic pathway, but these microorganisms are not of commercial relevance due to their inherent low performance characteristics, which include low productivity, low titer, low yield, and the requirement for oxygen during the fermentation process.

The present inventors have overcome these problems by developing metabolically engineered microorganisms that exhibit increased isobutanol productivity, titer, and/or yield.

### SUMMARY OF THE INVENTION

The present invention provides recombinant microorganisms that comprise an isobutanol producing metabolic pathway and methods of using said recombinant microorganisms to produce isobutanol.

In a first aspect, the invention provides recombinant microorganisms comprising an isobutanol producing metabolic pathway. In one embodiment, the isobutanol producing metabolic pathway comprises enzymes catalyzing the conversions (a-e): a) pyruvate to acetolactate; b) acetolactate to dihydroxyisovalerate; c) dihydroxy isovalerate to ketoisovalerate; d) ketoisovalerate to isobutyraldehyde; and e) isobutyraldehyde to isobutanol. In another embodiment, the isobutanol producing metabolic pathway comprises at least one of the enzymes selected from (a-f): a) acetolactate synthase (ALS); b) ketolacid reductoisomerase (KARI); c) dihydroxy acid dehydratese (DHAD); d) ketoisovalerate decarboxylase (KIVD); e) alcohol dehydrogenase (ADH); and f) branched chain amino acid aminotransferase.

In one embodiment, the recombinant microorganisms of the present invention comprise an isobutanol producing metabolic pathway with at least one isobutanol pathway enzyme localized in the mitochondria. In another embodiment, the recombinant microorganisms comprise an isobutanol producing metabolic pathway with at least two isobutanol pathway enzymes localized in the mitochondria. In another embodiment, the recombinant microorganisms comprise an isobutanol producing metabolic pathway with at least three isobutanol pathway enzymes localized in the mitochondria. In another embodiment, the recombinant microorganisms comprise an isobutanol producing metabolic pathway with at least four isobutanol pathway enzymes localized in the mitochondria. In yet another embodiment, the recombinant microorganisms comprise an isobutanol producing metabolic pathway with five isobutanol pathway enzymes localized in the mitochondria.

In one embodiment, the present invention provides a recombinant eukaryotic microorganism capable of producing isobutanol from a carbon source, said recombinant eukaryotic microorganism comprising an isobutanol producing metabolic pathway, wherein said metabolic pathway comprises enzymes catalyzing the conversions (a-e): a) pyruvate to acetolactate; b) acetolactate to dihydroxyisovalerate; c) dihydroxy isovalerate to ketoisovalerate; d) ketoisovalerate to isobutyraldehyde; and e) isobutyraldehyde to isobutanol, and wherein at least one of the conversions (a-e) occurs in the mitochondria. In one embodiment, the conversion is (a) pyruvate to acetolactate. In one embodiment, at least two of the conversions (a-e) occur in the mitochondria. In another embodiment, at least three of the conversions (a-e) occur in the mitochondria. In another embodiment, at least four of the conversions (a-e) occur in the mitochondria. In yet another embodiment, all five of the conversions (a-e) occur in the mitochondria.

In another embodiment, the present invention provides a recombinant eukaryotic microorganism capable of producing isobutanol from a carbon source, said recombinant eukaryotic microorganism comprising an isobutanol producing metabolic pathway, wherein said metabolic pathway comprises at least one of the enzymes selected from (a-f): a) acetolactate synthase (ALS); b) ketolacid reductoisomerase (KARI); c) dihydroxy acid dehydratese (DHAD); d) ketoisovalerate decarboxylase (KIVD); e) alcohol dehydrogenase (ADH); and a f) branched chain amino acid aminotransferase, and wherein at least one of the enzymes (a-f) is overexpressed and targeted to the mitochondria. In one embodiment, the enzyme is ALS. In one embodiment, at least two of the enzymes (a-f) are overexpressed and targeted to the mitochondria. In another embodiment, at least three of the enzymes (a-f) are overexpressed and targeted to the mitochondria. In another embodiment, at least four of the enzymes (a-f) are overexpressed and targeted to the mitochondria. In yet another embodiment, five of the enzymes are overexpressed and targeted to the mitochondria.

In another embodiment, the present invention provides methods of producing isobutanol using one or more recombinant microorganisms of the invention. In one embodiment, the method includes cultivating one or more recombinant microorganisms in a culture medium containing a feedstock providing the carbon source until a recoverable quantity of the isobutanol is produced and optionally, recovering the isobutanol. In one embodiment, the microorganism is selected to produce isobutanol from a carbon source at a yield of at least about 5 percent theoretical. In another embodiment, the microorganism is selected to produce isobutanol at a yield of at least about 10 percent, at least about 15 percent, about least about 20 percent, at least about 25 percent, at least about 30 percent, at least about 35 percent, at least about 40 percent, at least about 45 percent, at least about 50 percent, at least about 55 percent, at least about 60 percent, at least about 65 percent, at least about 70 percent, at least about 75 percent, or at least about 80 percent theoretical.

In one embodiment, the recombinant microorganisms of the invention produce isobutanol at a specific productivity of at least about 0.003 g/L/h/OD. In another embodiment, the microorganism produces isobutanol at a specific productivity of at least about 0.006 g/L/h/OD, at least about 0.009 g/L/h/OD, at least about 0.012 g/L/h/OD, at least about 0.015 g/L/h/OD, or at least about 0.020 g/L/h/OD.

In one embodiment, the recombinant microorganisms of the invention produce isobutanol at a titer of at least about 2.7 g/L. In another embodiment, the microorganism produces isobutanol at a total titer of at least about 4 g/L, at least about 6 g/L, at least about 8 g/L, at least about 10 g/L, or at least about 12 g/L.

In one embodiment, the recombinant microorganisms of the invention produce isobutanol at a total titer of at least about 21 g/L. In another embodiment, the microorganism produces isobutanol at a total titer of at least about 25 g/L, at least about 30 g/L, at least about 35 g/L, at least about 40 g/L, or at least about 50 g/L.

In one embodiment, the present invention provides a method of producing isobutanol, comprising the steps of (a) providing a recombinant microorganism comprising an isobutanol producing metabolic pathway with at least one isobutanol pathway enzyme localized in the mitochondria, wherein said recombinant microorganism is selected to produce isobutanol from a carbon source at a yield of at least about 5 percent theoretical; (b) cultivating said recombinant microorganism in a culture medium containing a feedstock providing the carbon source until a recoverable quantity of isobutanol is produced; and (c) recovering the isobutanol.

In another embodiment, the present invention provides a method of producing isobutanol, comprising the steps of (a) providing a recombinant microorganism comprising: (i) an isobutanol producing metabolic pathway with at least one isobutanol pathway enzyme localized in the mitochondria, wherein said recombinant microorganism is selected to produce isobutanol from a carbon source; and a (ii) metabolic pathway for the conversion of a carbon source to isobutanol which is at least partially balanced with respect to cofactor usage; (b) cultivating said recombinant microorganism in a culture medium containing a feedstock providing the carbon source until a recoverable quantity of isobutanol is produced; and (c) recovering the isobutanol.

In various embodiments described herein, the isobutanol pathway enzyme(s) is/are selected from the group consisting of acetolactate synthase (ALS), ketol-acid reductoisomerase (KARI), dihydroxyacid dehydratase (DHAD), 2-keto-acid decarboxylase (KIVD), and isobutyraldehyde dehydrogenase (IDH).

In some embodiments, the present invention provides recombinant microorganisms that have been engineered to express a heterologous metabolic pathway for conversion of pyruvate to isobutanol. In one embodiment, the recombinant microorganism is further engineered to increase the activity of a native metabolic pathway for conversion of pyruvate to isobutanol. In another embodiment, the recombinant microorganism is further engineered to include at least one enzyme encoded by a heterologous gene and at least one enzyme encoded by a native gene. In yet another embodiment, the recombinant microorganism is selected to include a native metabolic pathway for conversion of pyruvate to isobutanol. In yet another embodiment, the recombinant microorganism comprises a reduction in the activity of a native metabolic pathway as compared to a parental microorganism. In various embodiments described herein, one or more of the enzymes catalyzing the conversion of pyruvate to isobutanol is/are localized in the mitochondria.

In another embodiment, the microorganism may further be engineered to overexpress the BAT1 gene, BAT2 gene, or both the BAT1 and BAT2 genes.

In another embodiment, the recombinant microorganisms of the present invention may further be engineered to reduce ethanol production. In one embodiment, the recombinant microorganism may be further engineered to eliminate ethanol production. In one embodiment, the recombinant microorganism may further be engineered to include reduced pyruvate decarboxylase (PDC) activity as compared to a parental microorganism. In one embodiment, PDC activity is eliminated. PDC catalyzes the decarboxylation of pyruvate to acetaldehyde, which is reduced to ethanol by alcohol dehydrogenases via the oxidation of NADH to NAD+. In one embodiment, the recombinant microorganism includes a mutation in at least one PDC gene resulting in a reduction of PDC activity of a polypeptide encoded by said gene. In another embodiment, the recombinant microorganism includes a partial deletion of a PDC gene resulting in a reduction of PDC activity of a polypeptide encoded by said gene. In another embodiment, the recombinant microorganism comprises a complete deletion of a PDC gene resulting in a reduction of PDC activity of a polypeptide encoded by said gene. In yet another embodiment, the recombinant microorganism includes a modification of the regulatory region associated with at least one PDC gene resulting in a reduction of PDC activity of a polypeptide encoded by said gene. In yet another embodiment, the recombinant microorganism comprises a modification of the transcriptional regulator resulting in a reduction of PDC gene transcription. In yet another embodiment, the recombinant microorganism comprises mutations in all PDC genes resulting in a reduction of PDC activity of the polypeptides encoded by said genes.

In another embodiment, the recombinant microorganisms of the present invention may further be engineered to reduce glycerol production. In one embodiment the recombinant microorganism may be further engineered to eliminate glycerol production. In one embodiment, the recombinant microorganism may further be engineered to include reduced glycerol-3-phosphate dehydrogenase (GPD) activity as compared to a parental microorganism. In one embodiment, GPD activity is eliminated. GPD catalyzes the reduction of dihydroxyacetone phosphate (DHAP) to glycerol-3-phosphate (G3P) via the oxidation of NADH to NAD+. Glycerol is produced from G3P by Glycerol-3-phosphatase (GPP). In one embodiment, the recombinant microorganism includes a mutation in at least one GPD gene resulting in a reduction of GPD activity of a polypeptide encoded by said gene. In another embodiment, the recombinant microorganism includes a partial deletion of a GPD gene resulting in a reduction of GPD activity of a polypeptide encoded by the gene. In another embodiment, the recombinant microorganism comprises a complete deletion of a GPD gene resulting in a reduction of GPD activity of a polypeptide encoded by the gene. In yet another embodiment, the recombinant microorganism includes a modification of the regulatory region associated with at least one GPD gene resulting in a reduction of GPD activity of a polypeptide encoded by said gene. In yet another embodiment, the recombinant microorganism comprises a modification of the transcriptional regulator resulting in a reduction of GPD gene transcription. In yet another embodiment, the recombinant microorganism comprises mutations in all GPD genes resulting in a reduction of GPD activity of a polypeptide encoded by the gene.

In another embodiment, the recombinant microorganism is engineered with an isobutanol producing metabolic pathway that is at least partially balanced with respect to cofactor usage. In one embodiment, the isobutanol producing metabolic pathway is partially balanced with respect to cofactor usage by providing an NADH dependent alcohol dehydrogenase (ADH). In one embodiment, the NADH dependent ADH is encoded by DmADH from Drosophila melanogaster. In one embodiment, the NADH dependent ADH is encoded by adhA from Lactococcus lactis. In another embodiment, the isobutanol producing metabolic pathway is partially balanced with respect to cofactor usage by providing an NADH dependent ketol-acid reductoisomerase (KARI). In one embodiment, said KARI is engineered to have increased activity using NADH as the cofactor as compared to the S. cerevisiae Ilv5 protein and E. coli YqhD protein, respectively. In one embodiment, the NADH dependent KARI is encoded by EcilvCcoSc^{P2D1-A1-his6}.

In another embodiment, the recombinant microorganism is engineered with an isobutanol producing metabolic pathway that is balanced with respect to cofactor usage. In one embodiment, said metabolic pathway is balanced by providing an NADH dependent pathway for the conversion of pyruvate to isobutanol comprising an NADH dependent KARI enzyme and an NADH dependent ADH enzyme. In one embodiment, said KARI and said ADH are engineered to have increased activity using NADH as the cofactor as compared to the S. cerevisiae Ilv5 protein and E. coli YqhD protein, respectively. In another embodiment, said KARI and said ADH are identified in nature with increased activity using NADH as the cofactor as compared to the S. cerevisiae Ilv5 protein and E. coli YqhD protein, respectively.

In one embodiment, the metabolic pathway is balanced with respect to cofactor usage. In some embodiments, the metabolic pathway is balanced with respect to cofactor usage by the malate pathway. In additional embodiments, the metabolic pathway is balanced with respect to cofactor usage by the expression of a transhydrogenase. In some embodiments, the transhydrogenase is overexpressed. In one embodiment, the transhydrogenase is localized to the cytoplasmic membrane. In another embodiment, the transhydrogenase is localized to the mitochondrial membrane. In yet another embodiment, the transhydrogenase is localized to the mitochondrial membrane and the cytoplasmic membrane. In one embodiment, the transhydrogenase is a bacterial membrane bound transhydrogenase. In another embodiment, the transhydrogenase is a mammalian transhydrogenase. In yet another embodiment, the transhydrogenase is a fungal transhydrogenase. In an exemplary embodiment, the fungal transhydrogenase is derived from Neurospora crassa (GI:164426165).

In one embodiment, the metabolic pathway is balanced with respect to cofactor usage by providing an NADH dependent ADH and by the malate pathway. In another embodiment, the metabolic pathway is balanced with respect to cofactor usage by providing an NADH dependent ADH and the expression of a transhydrogenase.

In another embodiment, the recombinant microorganisms are further engineered to grow on glucose independently of C2-compounds at a growth rate substantially equivalent to the growth rate of a parental microorganism without altered PDC activity.

In various embodiments described herein, the microorganisms of the invention may produce isobutanol anaerobically under anaerobic conditions at a rate of at least about 10-fold higher than a parental microorganism comprising a native or unmodified metabolic pathway.

In another embodiment, the recombinant microorganism further comprises a pathway for the fermentation of isobutanol from a pentose sugar. In one embodiment, the pentose sugar is xylose. In one embodiment, the recombinant microorganism is engineered to express a functional xylose isomerase (XI). In another embodiment, the recombinant microorganism further comprises a deletion or disruption of a native gene encoding for an enzyme that catalyzes the conversion of xylose to xylitol. In one embodiment, the native gene is xylose reductase (XR). In another embodiment, the native gene is xylitol dehydrogenase (XDH). In yet another embodiment, both native genes are deleted or disrupted. In yet another embodiment, the recombinant microorganism further is engineered to overexpress either a heterologous or native gene encoding for an enzyme that catalyzes the conversion of xylulose to xylulose-5-phosphate.

In various embodiments described herein, the recombinant microorganisms may be microorganisms of the Saccharomyces clade, Saccharomyces sensu stricto group microorganisms, Crabtree-negative yeast microorganisms, Crabtree-positive yeast microorganisms, post-WGD (whole genome duplication) yeast microorganisms, pre-WGD (whole genome duplication) yeast microorganisms, and non-fermenting yeast microorganisms.

In some embodiments, the methods of the present invention utilize a yeast recombinant microorganism of the Saccharomyces clade.

In some embodiments, the methods of the present invention utilize a yeast recombinant microorganism of the Saccharomyces sensu stricto group.

In some embodiments, the methods of the present invention utilize a Crabtree-negative recombinant yeast microorganism. In one embodiment, the Crabtree-negative yeast microorganism is classified into a genera selected from the group consisting of Kluyveromyces, Pichia, Hansenula, or Candida. In additional embodiments, the Crabtree-negative yeast microorganism is selected from Kluyveromyces lactis, Kluyveromyces marxianus, Pichia anomala, Pichia stipitis, Pichia kudriavzevii, Hanensula anomala, Candida utilis and Kluyveromyces waltii.

In some embodiments, the methods of the present invention utilize a Crabtree-positive recombinant yeast microorganism. In one embodiment, the Crabtree-positive yeast microorganism is classified into a genera selected from the group consisting of Saccharomyces, Kluyveromyces, Zygosaccharomyces, Debaryomyces, Candida, Pichia and Schizosaccharomyces. In additional embodiments, the Crabtree-positive yeast microorganism is selected from the group consisting of Saccharomyces cerevisiae, Saccharomyces uvarum, Saccharomyces bayanus, Saccharomyces paradoxus, Saccharomyces castelli, Saccharomyces kluyveri, Kluyveromyces thermotolerans, Candida glabrata, Z. bailli, Z. rouxii, Debaryomyces hansenii, Pichia pastorius, Schizosaccharomyces pombe, and Saccharomyces uvarum.

In some embodiments, the methods of the present invention utilize a post-WGD (whole genome duplication) yeast recombinant microorganism. In one embodiment, the post-WGD yeast recombinant microorganism is classified into a genera selected from the group consisting of Saccharomyces or Candida. In additional embodiments, the post-WGD yeast is selected from the group consisting of Saccharomyces cerevisiae, Saccharomyces uvarum, Saccharomyces bayanus, Saccharomyces paradoxus, Saccharomyces castelli, and Candida glabrata.

In some embodiments, the methods of the present invention utilize a pre-WGD (whole genome duplication) yeast recombinant microorganism. In one embodiment, the pre-WGD yeast recombinant microorganism is classified into a genera selected from the group consisting of Saccharomyces, Kluyveromyces, Candida, Pichia, Debaryomyces, Hansenula, Pachysolen, Yarrowia and Schizosaccharomyces. In additional embodiments, the pre-WGD yeast is selected from the group consisting of Saccharomyces kluyveri, Kluyveromyces thermotolerans, Kluyveromyces marxianus, Kluyveromyces waltii, Kluyveromyces lactis, Candida tropicalis, Pichia pastoris, Pichia anomala, Pichia stipitis, Pichia kudriavzevii, Debaryomyces hansenii, Hansenula anomala, Pachysolen tannophilis, Yarrowia lipolytica, and Schizosaccharomyces pombe.

In some embodiments, the methods of the present invention utilize a microorganism that is a non-fermenting yeast microorganism, including, but not limited to those, classified into a genera selected from the group consisting of Tricosporon, Rhodotorula, or Myxozyma.

### BRIEF DESCRIPTION OF THE DRAWINGS

Illustrative embodiments of the invention are illustrated in the drawings, in which:

FIG. 1 illustrates an exemplary metabolic pathway for the conversion of glucose to isobutanol via pyruvate.

FIG. 2 illustrates an exemplary metabolic pathway for the mitochondrial conversion of glucose to isobutanol via pyruvate.

FIG. 3 illustrates the cofactor balance of an isobutanol producing yeast strain achieved by use of NADH dependent KARI and ADH.

FIG. 4 illustrates the cofactor balance of an isobutanol producing yeast strain achieved by use of NADH dependent KARI and ADH.

FIG. 5 illustrates the cofactor balance of an isobutanol producing yeast strain achieved by use of the malate bypass.

FIG. 6 illustrates the cofactor balance of an isobutanol producing yeast strain achieved by use of the malate bypass and a NADH dependent ADH.

FIG. 7 illustrates the cofactor balance of an isobutanol producing yeast strain achieved by use of a transhydrogenase.

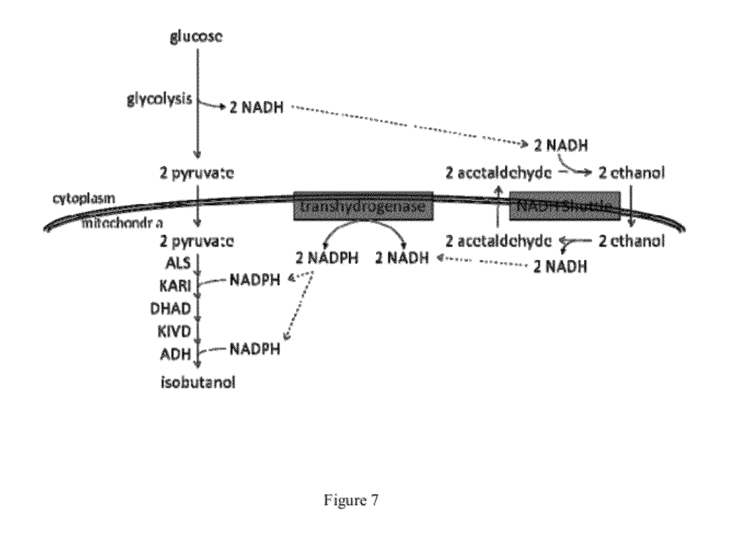

FIG. 8 illustrates the cofactor balance of an isobutanol producing yeast strain achieved by use of a transhydrogenase and a NADH dependent ADH.

FIG. 9 illustrates the results of fermentations using GEVO2062 and GEVO2072. Shown are isobutanol production, ethanol production, glucose consumption, and cell density (OD₆₀₀) over time. Shown are the averages and standard deviations of three replicate shake flasks for each strain.

FIG. 10 illustrates a proposed mitochondrial/cytosolic isobutanol pathway.

FIG. 11 shows the isobutanol production of four GEVO2072 transformant combinations.

FIGS. 12a-b illustrate titers of isobutanol (a) and (b) ethanol during shake flask fermentation experiments with GEVO1947 and eight transformants, including GEVO2087 (Transformant #10) and GEVO2088 (Transformant #7).

FIGS. 13a-b illustrate the production of (a) isobutanol and (b) ethanol in GEVO2087 and GEVO2072 compared to the negative control strains, GEVO1947 and GEVO1186, respectively.

FIG. 14 shows the isobutanol production in fermentations using GEVO2087, GEVO1969, GEVO2276, and GEVO2277. Isobutanol was measured after 0, 4.5, 8, 24, 49.5, and 71 hours incubation. The strains were GEVO2087 (PDC+, PATHWAY+), GEVO1969 (Pdc-minus, PATHWAY−), GEVO2276 (Pdc-minus, PATHWAY+, BsalsS-minus), and GEVO2277 (PDC+, PATHWAY+). The values are volume corrected.

### DETAILED DESCRIPTION

### Definitions

As used herein and in the appended claims, the singular forms “a,” “an,” and “the” include plural referents unless the context clearly dictates otherwise. Thus, for example, reference to “a polynucleotide” includes a plurality of such polynucleotides and reference to “the microorganism” includes reference to one or more microorganisms, and so forth.

Unless defined otherwise, all technical and scientific terms used herein have the same meaning as commonly understood to one of ordinary skill in the art to which this disclosure belongs. Although methods and materials similar or equivalent to those described herein can be used in the practice of the disclosed methods and compositions, the exemplary methods, devices and materials are described herein.

Any publications discussed above and throughout the text are provided solely for their disclosure prior to the filing date of the present application. Nothing herein is to be construed as an admission that the inventors are not entitled to antedate such disclosure by virtue of prior disclosure.

The term “microorganism” includes prokaryotic and eukaryotic microbial species from the Domains Archaea, Bacteria and Eucarya, the latter including yeast and filamentous fungi, protozoa, algae, or higher Protista. The terms “microbial cells” and “microbes” are used interchangeably with the term microorganism. The term “prokaryotes” is art recognized and refers to cells which contain no nucleus or other cell organelles. The prokaryotes are generally classified in one of two domains, the Bacteria and the Archaea. The definitive difference between organisms of the Archaea and Bacteria domains is based on fundamental differences in the nucleotide base sequence in the 16S ribosomal RNA.

The term “algae” means a eukaryotic microorganism that contains a chloroplast, and optionally that is capable of performing photosynthesis, or a prokaryotic microorganism capable of performing photosynthesis. Algae include obligate photoautotrophs, which cannot metabolize a fixed carbon source as energy, as well as heterotrophs, which can live solely off of a fixed carbon source. Algae can refer to unicellular organisms that separate from sister cells shortly after cell division, such as Chlamydomonas, and can also refer to microorganisms such as, for example, Volvox, which is a simple multicellular photosynthetic microbe of two distinct cell types. “Algae” can also refer to cells such as Chlorella and Dunaliella. “Algae” also includes other photosynthetic microorganisms that exhibit cell-cell adhesion, such as Agmenellum, Anabaena, and Pyrobotrys. “Algae” also includes obligate heterotrophic microorganisms that have lost the ability to perform photosynthesis, such as certain dinoflagellate algae species.

The term “Archaea” refers to a categorization of organisms of the division Mendosicutes, typically found in unusual environments and distinguished from the rest of the prokaryotes by several criteria, including the number of ribosomal proteins and the lack of muramic acid in cell walls. On the basis of ssrRNA analysis, the Archaea consist of two phylogenetically-distinct groups: Crenarchaeota and Euryarchaeota. On the basis of their physiology, the Archaea can be organized into three types: methanogens (prokaryotes that produce methane); extreme halophiles (prokaryotes that live at very high concentrations of salt (NaCl); and extreme (hyper) thermophilus (prokaryotes that live at very high temperatures). Besides the unifying archaeal features that distinguish them from Bacteria (i.e., no murein in cell wall, ester-linked membrane lipids, etc.), these prokaryotes exhibit unique structural or biochemical attributes which adapt them to their particular habitats. The Crenarchaeota consists mainly of hyperthermophilic sulfur-dependent prokaryotes and the Euryarchaeota contains the methanogens and extreme halophiles.

“Bacteria” or “eubacteria” refers to a domain of prokaryotic organisms. Bacteria include at least 11 distinct groups as follows: (1) Gram-positive (gram+) bacteria, of which there are two major subdivisions: (1) high G+C group (Actinomycetes, Mycobacteria, Micrococcus, others) (2) low G+C group (Bacillus, Clostridia, Lactobacillus, Staphylococci, Streptococci, Mycoplasmas); (2) Proteobacteria, e.g., Purple photosynthetic+non-photosynthetic Gram-negative bacteria (includes most “common” Gram-negative bacteria); (3) Cyanobacteria, e.g., oxygenic phototrophs; (4) Spirochetes and related species; (5) Planctomyces; (6) Bacteroides, Flavobacteria; (7) Chlamydia; (8) Green sulfur bacteria; (9) Green non-sulfur bacteria (also anaerobic phototrophs); (10) Radioresistant micrococci and relatives; (11) Thermotoga and Thermosipho thermophiles.

“Gram-negative bacteria” include cocci, nonenteric rods, and enteric rods. The genera of Gram-negative bacteria include, for example, Neisseria, Spirillum, Pasteurella, Brucella, Yersinia, Francisella, Haemophilus, Bordetella, Escherichia, Salmonella, Shigella, Klebsiella, Proteus, Vibrio, Pseudomonas, Bacteroides, Acetobacter, Aerobacter, Agrobacterium, Azotobacter, Spirilla, Serratia, Vibrio, Rhizobium, Chlamydia, Rickettsia, Treponema, and Fusobacterium.

“Gram positive bacteria” include cocci, nonsporulating rods, and sporulating rods. The genera of gram positive bacteria include, for example, Actinomyces, Bacillus, Clostridium, Corynebacterium, Erysipelothrix, Lactobacillus, Listeria, Mycobacterium, Myxococcus, Nocardia, Staphylococcus, Streptococcus, and Streptomyces.

The term “genus” is defined as a taxonomic group of related species according to the Taxonomic Outline of Bacteria and Archaea (Garrity, G. M., Lilburn, T. G., Cole, J. R., Harrison, S. H., Euzeby, J., and Tindall, B. J. (2007) The Taxonomic Outline of Bacteria and Archaea. TOBA Release 7.7, March 2007. Michigan State University Board of Trustees. [http://www.taxonomicoutline.org/]).

The term “species” is defined as a collection of closely related organisms with greater than 97% 16S ribosomal RNA sequence homology and greater than 70% genomic hybridization and sufficiently different from all other organisms so as to be recognized as a distinct unit.

The term “recombinant microorganism” and “recombinant host cell” are used interchangeably herein and refer to microorganisms that have been genetically modified to express or over-express endogenous polynucleotides, or to express heterologous polynucleotides, such as those included in a vector, or which have a reduction in expression of an endogenous gene. The polynucleotide generally encodes a target enzyme involved in a metabolic pathway for producing a desired metabolite. It is understood that the terms “recombinant microorganism” and “recombinant host cell” refer not only to the particular recombinant microorganism but to the progeny or potential progeny of such a microorganism. Because certain modifications may occur in succeeding generations due to either mutation or environmental influences, such progeny may not, in fact, be identical to the parent cell, but are still included within the scope of the term as used herein.

The term “wild-type microorganism” describes a cell that occurs in nature, i.e. a cell that has not been genetically modified. A wild-type microorganism can be genetically modified to express or overexpress a first target enzyme. This microorganism can act as a parental microorganism in the generation of a microorganism modified to express or overexpress a second target enzyme. In turn, the microorganism modified to express or overexpress a first and a second target enzyme can be modified to express or overexpress a third target enzyme.

Accordingly, a “parental microorganism” functions as a reference cell for successive genetic modification events. Each modification event can be accomplished by introducing a nucleic acid molecule in to the reference cell. The introduction facilitates the expression or over-expression of a target enzyme. It is understood that the term “facilitates” encompasses the activation of endogenous polynucleotides encoding a target enzyme through genetic modification of e.g., a promoter sequence in a parental microorganism. It is further understood that the term “facilitates” encompasses the introduction of heterologous polynucleotides encoding a target enzyme in to a parental microorganism.

The term “engineer” refers to any manipulation of a microorganism that result in a detectable change in the microorganism, wherein the manipulation includes but is not limited to inserting a polynucleotide and/or polypeptide heterologous to the microorganism and mutating a polynucleotide and/or polypeptide native to the microorganism.

As used herein, the term “metabolically engineered” or “metabolic engineering” involves rational pathway design and assembly of biosynthetic genes, genes associated with operons, and control elements of such polynucleotides, for the production of a desired metabolite. “Metabolically engineered” can further include optimization of metabolic flux by regulation and optimization of transcription, translation, protein stability and protein functionality using genetic engineering and appropriate culture condition including the reduction of, disruption, or knocking out of, a competing metabolic pathway that competes with an intermediate leading to a desired pathway.

The terms “metabolically engineered microorganism” and “modified microorganism” are used interchangeably herein and refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

The term “mutation” as used herein indicates any modification of a nucleic acid and/or polypeptide which results in an altered nucleic acid or polypeptide. Mutations include, for example, point mutations, deletions, or insertions of single or multiple residues in a polynucleotide, which includes alterations arising within a protein-encoding region of a gene as well as alterations in regions outside of a protein-encoding sequence, such as, but not limited to, regulatory or promoter sequences. A genetic alteration may be a mutation of any type. For instance, the mutation may constitute a point mutation, a frame-shift mutation, an insertion, or a deletion of part or all of a gene. In addition, in some embodiments of the modified microorganism, a portion of the microorganism genome has been replaced with a heterologous polynucleotide. In some embodiments, the mutations are naturally-occurring. In other embodiments, the mutations are the results of artificial mutation pressure. In still other embodiments, the mutations in the microorganism genome are the result of genetic engineering.

The term “biosynthetic pathway”, also referred to as “metabolic pathway”, refers to a set of anabolic or catabolic biochemical reactions for converting one chemical species into another. Gene products belong to the same “metabolic pathway” if they, in parallel or in series, act on the same substrate, produce the same product, or act on or produce a metabolic intermediate (i.e., metabolite) between the same substrate and metabolite end product.

The term “heterologous” as used herein with reference to molecules and in particular enzymes and polynucleotides, indicates molecules that are expressed in an organism other than the organism from which they originated or are found in nature, independently on the level of expression that can be lower, equal or higher than the level of expression of the molecule in the native microorganism.

On the other hand, the term “native” or “endogenous” as used herein with reference to molecules, and in particular enzymes and polynucleotides, indicates molecules that are expressed in the organism in which they originated or are found in nature, independently on the level of expression that can be lower equal or higher than the level of expression of the molecule in the native microorganism. It is understood that expression of native enzymes or polynucleotides may be modified in recombinant microorganisms.

The term “overexpressed” as used herein with reference to a gene indicates that the gene is expressed differently than in its native wild-type microorganism. Overexpressed genes include heterologous as well as native genes. Any engineering of a strain that leads to a change in the expression level of a native gene or that leads to a change in the regulation of said native gene renders said native gene “overexpressed.”

The term “mitochondrially targeted” as used herein refers to proteins that are completely or partially localized in the mitochondria of their host microorganism. This term can refer to proteins that are produced in their native form or it can refer to proteins that have been engineered to cause their mitochondrial localization.

The term “carbon source” generally refers to a substance suitable to be used as a source of carbon for eukaryotic cell growth. Carbon sources include, but are not limited to, biomass hydrolysates, starch, sucrose, cellulose, hemicellulose, xylose, and lignin, as well as monomeric components of these substrates. Carbon sources can comprise various organic compounds in various forms, including, but not limited to polymers, carbohydrates, acids, alcohols, aldehydes, ketones, amino acids, peptides, etc. These include, for example, various monosaccharides such as glucose, dextrose (D-glucose), maltose, oligosaccharides, polysaccharides, saturated or unsaturated fatty acids, succinate, lactate, acetate, ethanol, etc., or mixtures thereof. Photosynthetic organisms can additionally produce a carbon source as a product of photosynthesis. In some embodiments, carbon sources may be selected from biomass hydrolysates and glucose.

As used herein, the term “C2-compound” refers to organic compounds comprised of two carbon atoms, including but not limited to ethanol and acetate. As used herein, a “C2-independent organism” encompasses organisms that do not require ethanol or acetate for growth. Such a C2-independent organism may grow on glucose.

The term “feedstock” is defined as a raw material or mixture of raw materials supplied to a microorganism or fermentation process from which other products can be made. For example, a carbon source, such as biomass or the carbon compounds derived from biomass are a feedstock for a microorganism that produces a biofuel in a fermentation process. However, a feedstock may contain nutrients other than a carbon source.

The term “substrate” or “suitable substrate” refers to any substance or compound that is converted or meant to be converted into another compound by the action of an enzyme. The term includes not only a single compound, but also combinations of compounds, such as solutions, mixtures and other materials which contain at least one substrate, or derivatives thereof. Further, the term “substrate” encompasses not only compounds that provide a carbon source suitable for use as a starting material, such as any biomass derived sugar, but also intermediate and end product metabolites used in a pathway associated with a metabolically engineered microorganism as described herein.

The term “fermentation” or “fermentation process” is defined as a process in which a microorganism is cultivated in a culture medium containing raw materials, such as feedstock and nutrients, wherein the microorganism converts raw materials, such as a feedstock, into products.

The term “cell dry weight” or “CDW” refers to the weight of the microorganism after the water contained in the microorganism has been removed using methods known to one skilled in the art. CDW is reported in g/L.

The term “biofuel” refers to a fuel in which all carbon contained within the fuel is derived from biomass and is biochemically converted, at least in part, in to a fuel by a microorganism. A biofuel is further defined as a non-ethanol compound which contains less than 0.5 oxygen atoms per carbon atom. A biofuel is a fuel in its own right, but may be blended with petroleum-derived fuels to generate a fuel. A biofuel may be used as a replacement for petrochemically-derived gasoline, diesel fuel, or jet fuel.

The term “volumetric productivity” or “production rate” is defined as the amount of product formed per volume of medium per unit of time. Volumetric productivity is reported in gram per liter per hour (g/L/h).

The term “specific productivity” is defined as the rate of formation of the product. To describe productivity as an inherent parameter of the microorganism and not of the fermentation process, productivity is herein further defined as the specific productivity in gram product per gram of cell dry weight (CDW) per hour (g/g CDW/h). Using the relation of CDW to OD₆₀₀ for the given microorganism specific productivity can also be expressed as gram product per liter culture medium per optical density of the culture broth at 600 nm (OD) per hour (g/L/h/OD)

The term “yield” is defined as the amount of product obtained per unit weight of raw material and may be expressed as g product per g substrate (g/g). Yield may be expressed as a percentage of the theoretical yield. “Theoretical yield” is defined as the maximum amount of product that can be generated per a given amount of substrate as dictated by the stoichiometry of the metabolic pathway used to make the product. For example, the theoretical yield for one typical conversion of glucose to isobutanol is 0.41 g/g. As such, a yield of butanol from glucose of 0.39 g/g would be expressed as 95% of theoretical or 95% theoretical yield.

The term “titre” or “titer” is defined as the strength of a solution or the concentration of a substance in solution. For example, the titre of a biofuel in a fermentation broth is described as g of biofuel in solution per liter of fermentation broth (g/L).

The term “total titer” is defined as the sum of all biofuel produced in a process, including but not limited to the biofuel in solution, the biofuel in gas phase, and any biofuel removed from the process and recovered relative to the initial volume in the process or the operating volume in the process.

A “facultative anaerobic organism” or a “facultative anaerobic microorganism” is defined as an organism that can grow in either the presence or in the absence of oxygen.

A “strictly anaerobic organism” or a “strictly anaerobic microorganism” is defined as an organism that cannot grow in the presence of oxygen and which does not survive exposure to any concentration of oxygen.

An “anaerobic organism” or an “anaerobic microorganism” is defined as an organism that cannot grow in the presence of oxygen.

“Aerobic conditions” are defined as conditions under which the oxygen concentration in the fermentation medium is sufficiently high for an aerobic or facultative anaerobic microorganism to use as a terminal electron acceptor.

In contrast, “Anaerobic conditions” are defined as conditions under which the oxygen concentration in the fermentation medium is too low for the microorganism to use as a terminal electron acceptor. Anaerobic conditions may be achieved by sparging a fermentation medium with an inert gas such as nitrogen until oxygen is no longer available to the microorganism as a terminal electron acceptor. Alternatively, anaerobic conditions may be achieved by the microorganism consuming the available oxygen of the fermentation until oxygen is unavailable to the microorganism as a terminal electron acceptor.

“Dissolved oxygen,” abbreviated as “DO” is expressed throughout as the percentage of saturating concentration of oxygen in water.

“Aerobic metabolism” refers to a biochemical process in which oxygen is used as a terminal electron acceptor to make energy, typically in the form of ATP, from carbohydrates. Aerobic metabolism occurs e.g. via glycolysis and the TCA cycle, wherein a single glucose molecule is metabolized completely into carbon dioxide in the presence of oxygen.

In contrast, “anaerobic metabolism” refers to a biochemical process in which oxygen is not the final acceptor of electrons contained in NADH. Anaerobic metabolism can be divided into anaerobic respiration, in which compounds other than oxygen serve as the terminal electron acceptor, and substrate level phosphorylation, in which the electrons from NADH are utilized to generate a reduced product via a “fermentative pathway.”

In “fermentative pathways”, NAD(P)H donates its electrons to a molecule produced by the same metabolic pathway that produced the electrons carried in NAD(P)H. For example, in one of the fermentative pathways of certain yeast strains, NAD(P)H generated through glycolysis transfers its electrons to pyruvate, yielding lactate. Fermentative pathways are usually active under anaerobic conditions but may also occur under aerobic conditions, under conditions where NADH is not fully oxidized via the respiratory chain. For example, above certain glucose concentrations, Crabtree positive yeasts produce large amounts of ethanol under aerobic conditions.

The term “fermentation product” means any main product plus its coupled product. A “coupled product” is produced as part of the stoichiometric conversion of the carbon source to the main fermentation product. An example for a coupled product is the two molecules of CO₂ that are produced with every molecule of isobutanol during production of isobutanol from glucose according the biosynthetic pathway described herein.

The term “byproduct” means an undesired product related to the production of a biofuel. Byproducts are generally disposed as waste, adding cost to a biofuel production process.

The term “co-product” means a secondary or incidental product related to the production of biofuel. Co-products have potential commercial value that increases the overall value of biofuel production, and may be the deciding factor as to the viability of a particular biofuel production process.

The term “non-fermenting yeast” is a yeast species that fails to demonstrate an anaerobic metabolism in which the electrons from NADH are utilized to generate a reduced product via a fermentative pathway such as the production of ethanol and CO₂ from glucose. Non-fermentative yeast can be identified by the “Durham Tube Test” (J. A. Barnett, R. W. Payne, and D. Yarrow. 2000. Yeasts Characteristics and Identification. 3^{rd} edition. p. 28-29. Cambridge University Press, Cambridge, UK.) or by monitoring the production of fermentation productions such as ethanol and CO₂.

The term “polynucleotide” is used herein interchangeably with the term “nucleic acid” and refers to an organic polymer composed of two or more monomers including nucleotides, nucleosides or analogs thereof, including but not limited to single stranded or double stranded, sense or antisense deoxyribonucleic acid (DNA) of any length and, where appropriate, single stranded or double stranded, sense or antisense ribonucleic acid (RNA) of any length, including siRNA. The term “nucleotide” refers to any of several compounds that consist of a ribose or deoxyribose sugar joined to a purine or a pyrimidine base and to a phosphate group, and that are the basic structural units of nucleic acids. The term “nucleoside” refers to a compound (as guanosine or adenosine) that consists of a purine or pyrimidine base combined with deoxyribose or ribose and is found especially in nucleic acids. The term “nucleotide analog” or “nucleoside analog” refers, respectively, to a nucleotide or nucleoside in which one or more individual atoms have been replaced with a different atom or with a different functional group. Accordingly, the term polynucleotide includes nucleic acids of any length, DNA, RNA, analogs and fragments thereof. A polynucleotide of three or more nucleotides is also called nucleotidic oligomer or oligonucleotide.

It is understood that the polynucleotides described herein include “genes” and that the nucleic acid molecules described herein include “vectors” or “plasmids.” Accordingly, the term “gene”, also called a “structural gene” refers to a polynucleotide that codes for a particular sequence of amino acids, which comprise all or part of one or more proteins or enzymes, and may include regulatory (non-transcribed) DNA sequences, such as promoter sequences, which determine for example the conditions under which the gene is expressed. The transcribed region of the gene may include untranslated regions, including introns, 5′-untranslated region (UTR), and 3′-UTR, as well as the coding sequence.

The term “expression” with respect to a gene sequence refers to transcription of the gene and, as appropriate, translation of the resulting mRNA transcript to a protein. Thus, as will be clear from the context, expression of a protein results from transcription and translation of the open reading frame sequence.

The term “operon” refers two or more genes which are transcribed as a single transcriptional unit from a common promoter. In some embodiments, the genes comprising the operon are contiguous genes. It is understood that transcription of an entire operon can be modified (i.e., increased, decreased, or eliminated) by modifying the common promoter. Alternatively, any gene or combination of genes in an operon can be modified to alter the function or activity of the encoded polypeptide. The modification can result in an increase in the activity of the encoded polypeptide. Further, the modification can impart new activities on the encoded polypeptide. Exemplary new activities include the use of alternative substrates and/or the ability to function in alternative environmental conditions.

In various embodiments described herein, NAD(P)H refers to either NADH or NADPH. NAD(P)⁺ refers to either NAD⁺ or NADP⁺.

A “vector” is any means by which a nucleic acid can be propagated and/or transferred between organisms, cells, or cellular components. Vectors include viruses, bacteriophage, pro-viruses, plasmids, phagemids, transposons, and artificial chromosomes such as YACs (yeast artificial chromosomes), BACs (bacterial artificial chromosomes), and PLACs (plant artificial chromosomes), and the like, that are “episomes,” that is, that replicate autonomously or can integrate into a chromosome of a host cell. A vector can also be a naked RNA polynucleotide, a naked DNA polynucleotide, a polynucleotide composed of both DNA and RNA within the same strand, a poly-lysine-conjugated DNA or RNA, a peptide-conjugated DNA or RNA, a liposome-conjugated DNA, or the like, that are not episomal in nature, or it can be an organism which comprises one or more of the above polynucleotide constructs such as an agrobacterium or a bacterium.

“Transformation” refers to the process by which a vector is introduced into a host cell. Transformation (or transduction, or transfection), can be achieved by any one of a number of means including electroporation, microinjection, biolistics (or particle bombardment-mediated delivery), or agrobacterium mediated transformation.

The term “enzyme” as used herein refers to any substance that catalyzes or promotes one or more chemical or biochemical reactions, which usually includes enzymes totally or partially composed of a polypeptide, but can include enzymes composed of a different molecule including polynucleotides.

The term “protein” or “polypeptide” as used herein indicates an organic polymer composed of two or more amino acidic monomers and/or analogs thereof. As used herein, the term “amino acid” or “amino acidic monomer” refers to any natural and/or synthetic amino acids including glycine and both D or L optical isomers. The term “amino acid analog” refers to an amino acid in which one or more individual atoms have been replaced, either with a different atom, or with a different functional group. Accordingly, the term polypeptide includes amino acidic polymer of any length including full length proteins, and peptides as well as analogs and fragments thereof. A polypeptide of three or more amino acids is also called a protein oligomer or oligopeptide

The term “homologs” used with respect to an original enzyme or gene of a first family or species refers to distinct enzymes or genes of a second family or species which are determined by functional, structural or genomic analyses to be an enzyme or gene of the second family or species which corresponds to the original enzyme or gene of the first family or species. Most often, homologs will have functional, structural or genomic similarities. Techniques are known by which homologs of an enzyme or gene can readily be cloned using genetic probes and PCR. Identity of cloned sequences as homolog can be confirmed using functional assays and/or by genomic mapping of the genes.

A protein has “homology” or is “homologous” to a second protein if the nucleic acid sequence that encodes the protein has a similar sequence to the nucleic acid sequence that encodes the second protein. Alternatively, a protein has homology to a second protein if the two proteins have “similar” amino acid sequences. (Thus, the term “homologous proteins” is defined to mean that the two proteins have similar amino acid sequences).

The term “analog” or “analogous” refers to nucleic acid or protein sequences or protein structures that are related to one another in function only and are not from common descent or do not share a common ancestral sequence. Analogs may differ in sequence but may share a similar structure, due to convergent evolution. For example, two enzymes are analogs or analogous if the enzymes catalyze the same reaction of conversion of a substrate to a product, are unrelated in sequence, and irrespective of whether the two enzymes are related in structure.

### The Microorganism in General

Microorganism Characterized by Producing Isobutanol from Pyruvate Via an Overexpressed Isobutanol Pathway

Native producers of 1-butanol, such as Clostridium acetobutylicum, are known, but these organisms also generate byproducts such as acetone, ethanol, and butyrate during fermentations. Furthermore, these microorganisms are relatively difficult to manipulate, with significantly fewer tools available than in more commonly used production hosts such as E. coli. Additionally, the physiology and metabolic regulation of these native producers are much less well understood, impeding rapid progress towards high-efficiency production. Furthermore, no native microorganisms have been identified that can metabolize glucose into isobutanol in industrially relevant quantities.

The production of isobutanol and other fusel alcohols by various yeast species, including Saccharomyces cerevisiae is of special interest to the distillers of alcoholic beverages, for whom fusel alcohols constitute often undesirable off-notes. Production of isobutanol in wild-type yeasts has been documented on various growth media, ranging from grape must from winemaking (Romano, et al., Metabolic diversity of Saccharomyces cerevisiae strains from spontaneously fermented grape musts, 19:311-315, 2003), in which 12-219 mg/L isobutanol were produced, to supplemented minimal media (Oliviera, et al. (2005) World Journal of Microbiology and Biotechnology 21:1569-1576), producing 16-34 mg/L isobutanol. Work from Dickinson, et al. (J Biol. Chem. 272(43):26871-8, 1997) has identified the enzymatic steps utilized in an endogenous S. cerevisiae pathway converting branch-chain amino acids (e.g., valine or leucine) to isobutanol.

Recombinant microorganisms provided herein can express a plurality of heterologous and/or native target enzymes involved in pathways for the production isobutanol from a suitable carbon source.

Accordingly, metabolically “engineered” or “modified” microorganisms are produced via the introduction of genetic material into a host or parental microorganism of choice and/or by modification of the expression of native genes, thereby modifying or altering the cellular physiology and biochemistry of the microorganism. Through the introduction of genetic material and/or the modification of the expression of native genes the parental microorganism acquires new properties, e.g. the ability to produce a new, or greater quantities of, an intracellular metabolite. As described herein, the introduction of genetic material and/or the modification of the expression of native genes into a parental microorganism results in a new or modified ability to produce isobutanol. The genetic material introduced into and/or the genes modified for expression in the parental microorganism contains gene(s), or parts of genes, coding for one or more of the enzymes involved in a biosynthetic pathway for the production of isobutanol and may also include additional elements for the expression and/or regulation of expression of these genes, e.g. promoter sequences.

An engineered or modified microorganism can also include in the alternative or in addition to the introduction of a genetic material into a host or parental microorganism, the disruption, deletion or knocking out of a gene or polynucleotide to alter the cellular physiology and biochemistry of the microorganism. Through the reduction, disruption or knocking out of a gene or polynucleotide the microorganism acquires new or improved properties (e.g., the ability to produce a new metabolite or greater quantities of an intracellular metabolite, improve the flux of a metabolite down a desired pathway, and/or reduce the production of undesirable by-products).

Recombinant microorganisms provided herein may also produce metabolites in quantities not available in the parental microorganism. A “metabolite” refers to any substance produced by metabolism or a substance necessary for or taking part in a particular metabolic process. A metabolite can be an organic compound that is a starting material (e.g., glucose or pyruvate), an intermediate (e.g., 2-ketoisovalerate), or an end product (e.g., isobutanol) of metabolism. Metabolites can be used to construct more complex molecules, or they can be broken down into simpler ones. Intermediate metabolites may be synthesized from other metabolites, perhaps used to make more complex substances, or broken down into simpler compounds, often with the release of chemical energy.

Exemplary metabolites include glucose, pyruvate, and isobutanol. The metabolite isobutanol can be produced by a recombinant microorganism metabolically engineered to express or over-express a metabolic pathway that converts pyruvate to isobutanol. An exemplary metabolic pathway that converts pyruvate to isobutanol may be comprised of a acetohydroxy acid synthase (ALS) enzyme encoded by, for example, BsalsS from B. subtilis, a ketolacid reductoisomerase (KARI) encoded by, for example ilvC from E. coli, a dihydroxy-acid dehydratase (DHAD), encoded by, for example ilvD from E. coli, a 2-keto-acid decarboxylase (KIVD) encoded by, for example LlkivD (SEQ ID NO: 153) or LlkivD2 (SEQ ID NO: 155) from L. lactis, and an isobutyraldehyde dehydrogenase (IDH), encoded by, for example, a native S. cerevisiae alcohol dehydrogenase gene like ADH7.

Accordingly, provided herein are recombinant microorganisms that produce isobutanol and in some aspects may include the elevated expression of target enzymes such as ALS (encoded e.g. by ILV2 and ILV6 from Saccharomyces cerevisiae or BsalsS from Bacillus subtilis), KARI (encoded e.g. by ILV5 from S. cerevisiae or ilvC from E. coli), DHAD (encoded, e.g. by ILV3 from S. cerevisiae or by ilvD from E. coli), and KIVD (encoded, e.g. by PDC1, PDC5 and PDC6 from S. cerevisiae, ARO10 from S. cerevisiae, THI3 from S. cerevisiae, LlkivD from L. lactis, or pdc from Z. mobilis).

The disclosure identifies specific genes useful in the methods, compositions and organisms of the disclosure; however it will be recognized that absolute identity to such genes is not necessary. For example, changes in a particular gene or polynucleotide comprising a sequence encoding a polypeptide or enzyme can be performed and screened for activity. Typically such changes comprise conservative mutation and silent mutations. Such modified or mutated polynucleotides and polypeptides can be screened for expression of a functional enzyme using methods known in the art.

Due to the inherent degeneracy of the genetic code, other polynucleotides which encode substantially the same or a functionally equivalent polypeptide can also be used to clone and express the polynucleotides encoding such enzymes.

As will be understood by those of skill in the art, it can be advantageous to modify a coding sequence to enhance its expression in a particular host. The genetic code is redundant with 64 possible codons, but most organisms typically use a subset of these codons. The codons that are utilized most often in a species are called optimal codons, and those not utilized very often are classified as rare or low-usage codons. Codons can be substituted to reflect the preferred codon usage of the host, a process sometimes called “codon optimization” or “controlling for species codon bias.”

Optimized coding sequences containing codons preferred by a particular prokaryotic or eukaryotic host (see also, Murray et al. (1989) Nucl. Acids Res. 17:477-508) can be prepared, for example, to increase the rate of translation or to produce recombinant RNA transcripts having desirable properties, such as a longer half-life, as compared with transcripts produced from a non-optimized sequence. Translation stop codons can also be modified to reflect host preference. For example, typical stop codons for S. cerevisiae and mammals are UAA and UGA, respectively. The typical stop codon for monocotyledonous plants is UGA, whereas insects and E. coli commonly use UAA as the stop codon (Dalphin et al. (1996) Nucl. Acids Res. 24: 216-218). Methodology for optimizing a nucleotide sequence for expression in a plant is provided, for example, in U.S. Pat. No. 6,015,891, and the references cited therein.

Those of skill in the art will recognize that, due to the degenerate nature of the genetic code, a variety of DNA compounds differing in their nucleotide sequences can be used to encode a given enzyme of the disclosure. The native DNA sequence encoding the biosynthetic enzymes described above are referenced herein merely to illustrate an embodiment of the disclosure, and the disclosure includes DNA compounds of any sequence that encode the amino acid sequences of the polypeptides and proteins of the enzymes utilized in the methods of the disclosure. In similar fashion, a polypeptide can typically tolerate one or more amino acid substitutions, deletions, and insertions in its amino acid sequence without loss or significant loss of a desired activity. The disclosure includes such polypeptides with different amino acid sequences than the specific proteins described herein so long as they modified or variant polypeptides have the enzymatic anabolic or catabolic activity of the reference polypeptide. Furthermore, the amino acid sequences encoded by the DNA sequences shown herein merely illustrate embodiments of the disclosure.

In addition, homologs of enzymes useful for generating metabolites are encompassed by the microorganisms and methods provided herein.

As used herein, two proteins (or a region of the proteins) are substantially homologous when the amino acid sequences have at least about 30%, 40%, 50% 60%, 65%, 70%, 75%, 80%, 85%, 90%, 91%, 92%, 93%, 94%, 95%, 96%, 97%, 98%, or 99% identity. To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In one embodiment, the length of a reference sequence aligned for comparison purposes is at least 30%, typically at least 40%, more typically at least 50%, even more typically at least 60%, and even more typically at least 70%, 80%, 90%, 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

When “homologous” is used in reference to proteins or peptides, it is recognized that residue positions that are not identical often differ by conservative amino acid substitutions. A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. In cases where two or more amino acid sequences differ from each other by conservative substitutions, the percent sequence identity or degree of homology may be adjusted upwards to correct for the conservative nature of the substitution. Means for making this adjustment are well known to those of skill in the art (see, e.g., Pearson et al., 1994, hereby incorporated herein by reference).

The following six groups each contain amino acids that are conservative substitutions for one another: 1) Serine (S), Threonine (T); 2) Aspartic Acid (D), Glutamic Acid (E); 3) Asparagine (N), Glutamine (Q); 4) Arginine (R), Lysine (K); 5) Isoleucine (I), Leucine (L), Methionine (M), Alanine (A), Valine (V), and 6) Phenylalanine (F), Tyrosine (Y), Tryptophan (W).

Sequence homology for polypeptides, which is also referred to as percent sequence identity, is typically measured using sequence analysis software. See, e.g., the Sequence Analysis Software Package of the Genetics Computer Group (GCG), University of Wisconsin Biotechnology Center, 910 University Avenue, Madison, Wis. 53705. Protein analysis software matches similar sequences using measure of homology assigned to various substitutions, deletions and other modifications, including conservative amino acid substitutions. For instance, GCG contains programs such as “Gap” and “Bestfit” which can be used with default parameters to determine sequence homology or sequence identity between closely related polypeptides, such as homologous polypeptides from different species of organisms or between a wild-type protein and a mutant protein. See, e.g., GCG Version 6.1.

A typical algorithm used comparing a molecule sequence to a database containing a large number of sequences from different organisms is the computer program BLAST (Altschul, S. F., et al. (1990) “Basic local alignment search tool.” J. Mol. Biol. 215:403-410; Gish, W. and States, D. J. (1993) “Identification of protein coding regions by database similarity search.” Nature Genet. 3:266-272; Madden, T. L., et al. (1996) “Applications of network BLAST server” Meth. Enzymol. 266:131-141; Altschul, S. F., et al. (1997) “Gapped BLAST and PSI-BLAST: a new generation of protein database search programs.” Nucleic Acids Res. 25:3389-3402; Zhang, J. and Madden, T. L. (1997) “PowerBLAST: A new network BLAST application for interactive or automated sequence analysis and annotation.” Genome Res. 7:649-656), especially blastp or tblastn (Altschul, S. F., et al. (1997) “Gapped BLAST and PSI-BLAST: a new generation of protein database search programs.” Nucleic Acids Res. 25:3389-3402). Typical parameters for BLASTp are: Expectation value: 10 (default); Filter: seg (default); Cost to open a gap: 11 (default); Cost to extend a gap: 1 (default); Max. alignments: 100 (default); Word size: 11 (default); No. of descriptions: 100 (default); Penalty Matrix: BLOSUM62.

When searching a database containing sequences from a large number of different organisms, it is typical to compare amino acid sequences. Database searching using amino acid sequences can be measured by algorithms other than blastp known in the art. For instance, polypeptide sequences can be compared using FASTA, a program in GCG Version 6.1. FASTA provides alignments and percent sequence identity of the regions of the best overlap between the query and search sequences (Pearson, W. R. (1990) “Rapid and Sensitive Sequence Comparison with FASTP and FASTA” Meth. Enzymol. 183:63-98). For example, percent sequence identity between amino acid sequences can be determined using FASTA with its default parameters (a word size of 2 and the PAM250 scoring matrix), as provided in GCG Version 6.1, hereby incorporated herein by reference.

The disclosure provides metabolically engineered microorganisms comprising a biochemical pathway for the production of isobutanol from a suitable substrate at a high yield. A metabolically engineered microorganism of the disclosure comprises one or more recombinant polynucleotides within the genome of the organism or external to the genome within the organism. The microorganism can comprise a reduction, disruption or knockout of a gene found in the wild-type organism and/or introduction of a heterologous polynucleotide and/or expression or overexpression of an endogenous polynucleotide.

In one aspect, the disclosure provides a recombinant microorganism comprising elevated or altered expression of at least one target enzyme as compared to a parental microorganism or encodes an enzyme not found in the parental organism. In another or further aspect, the microorganism comprises a reduction, disruption or knockout of at least one gene encoding an enzyme that competes with a metabolite necessary for the production of isobutanol. The recombinant microorganism produces at least one metabolite involved in a biosynthetic pathway for the production of isobutanol. In general, the recombinant microorganisms comprises at least one recombinant metabolic pathway that comprises a target enzyme and may further include a reduction in activity or expression of an enzyme in a competitive biosynthetic pathway. The pathway acts to modify a substrate or metabolic intermediate in the production of isobutanol. The target enzyme is encoded by, and expressed from, a polynucleotide derived from a suitable biological source. In some embodiments, the polynucleotide comprises a gene derived from a prokaryotic or eukaryotic source and recombinantly engineered into the microorganism of the disclosure. In other embodiments, the polynucleotide comprises a gene that is native to the host organism.

It is understood that a range of microorganisms can be modified to include a recombinant metabolic pathway suitable for the production of isobutanol. In various embodiments, microorganisms may be selected from yeast microorganisms. Yeast microorganisms for the production of isobutanol at high yield may be selected based on certain characteristics:

Another characteristic may include the property that the microorganism is selected to convert various carbon sources into isobutanol. Accordingly, in one embodiment, the recombinant microorganism herein disclosed can convert a variety of carbon sources to products, including but not limited to glucose, galactose, mannose, xylose, arabinose, lactose, sucrose, and mixtures thereof.

The recombinant microorganism may thus further include a pathway for the fermentation of isobutanol from five-carbon (pentose) sugars including xylose. Most yeast species metabolize xylose via a complex route, in which xylose is first reduced to xylitol via a xylose reductase (XR) enzyme. The xylitol is then oxidized to xylulose via a xylitol dehydrogenase (XDH) enzyme. The xylulose is then phosphorylated via a xylulokinase (XK) enzyme. This pathway operates inefficiently in yeast species because it introduces a redox imbalance in the cell. The xylose-to-xylitol step uses NADH as a cofactor, whereas the xylitol-to-xylulose step uses NADPH as a cofactor. Other processes must operate to restore the redox imbalance within the cell. This often means that the organism cannot grow anaerobically on xylose or other pentose sugar. Accordingly, a yeast species that can efficiently ferment xylose and other pentose sugars into a desired fermentation product is therefore very desirable.

Thus, in one aspect, the recombinant is engineered to express a functional exogenous xylose isomerase. Exogenous xylose isomerases functional in yeast are known in the art. See, e.g., Rajgarhia et al, US20060234364, which is herein incorporated by reference in its entirety. In an embodiment according to this aspect, the exogenous xylose isomerase gene is operatively linked to promoter and terminator sequences that are functional in the yeast cell. In a preferred embodiment, the recombinant microorganism further has a deletion or disruption of a native gene that encodes for an enzyme (e.g. XR and/or XDH) that catalyzes the conversion of xylose to xylitol. In a further preferred embodiment, the recombinant microorganism also contains a functional, exogenous xylulokinase (XK) gene operatively linked to promoter and terminator sequences that are functional in the yeast cell. In one embodiment, the xylulokinase (XK) gene is overexpressed.

Another characteristic may include the property that the wild-type or parental microorganism is non-fermenting. In other words, it cannot metabolize a carbon source anaerobically while the yeast is able to metabolize a carbon source in the presence of oxygen. Non-fermenting yeast refers to both naturally occurring yeasts as well as genetically modified yeast. During anaerobic fermentation with fermentative yeast, the main pathway to oxidize the NADH from glycolysis is through the production of ethanol. Ethanol is produced by alcohol dehydrogenase (ADH) via the reduction of acetaldehyde, which is generated from pyruvate by pyruvate decarboxylase (PDC). Thus, in one embodiment, a fermentative yeast can be engineered to be non-fermentative by the reduction or elimination of the native PDC activity. Thus, most of the pyruvate produced by glycolysis is not consumed by PDC and is available for the isobutanol pathway. Deletion of this pathway increases the pyruvate and the reducing equivalents available for the isobutanol pathway. Fermentative pathways contribute to low yield and low productivity of isobutanol. Accordingly, deletion of PDC may increase yield and productivity of isobutanol. In one embodiment, the yeast microorganisms may be selected from the “Saccharomyces Yeast Clade”, defined as an ascomycetous yeast taxonomic class by Kurtzman and Robnett in 1998 (“Identification and phylogeny of ascomycetous yeast from analysis of nuclear large subunit (26S) ribosomal DNA partial sequences.” Antonie van Leeuwenhoek 73: 331-371). They were able to determine the relatedness of yeast of approximately 500 yeast species by comparing the nucleotide sequence of the D1/D2 domain at the 5′ end of the gene encoding the large ribosomal subunit 26S. In pair-wise comparisons of the D1/D2 nucleotide sequence of S. cerevisiae and the two most distant yeast in the Saccharomyces clade: K. lactis and K. marxianus, yeast from this clade share greater than 80% identity.

The term “Saccharomyces sensu stricto” taxonomy group is a cluster of yeast species that are highly related to S. cerevisiae (Rainieri, S. et al 2003. Saccharomyces Sensu Stricto: Systematics, Genetic Diversity and Evolution. J. Biosci Bioengin 96(1)1-9. Saccharomyces sensu stricto yeast species include but are not limited to S. cerevisiae, S. cerevisiae, S. kudriavzevii, S. mikatae, S. bayanus, S. uvarum, S. carocanis and hybrids derived from these species (Masneuf et al. 1998. New Hybrids between Saccharomyces Sensu Stricto Yeast Species Found Among Wine and Cider Production Strains. Yeast 7(1)61-72).

An ancient whole genome duplication (WGD) event occurred during the evolution of hemiascomycete yeast was discovered using comparative genomics tools (Kellis et al 2004 “Proof and evolutionary analysis of ancient genome duplication in the yeast S. cerevisiae.” Nature 428:617-624. Dujon et al 2004 “Genome evolution in yeasts.” Nature 430:35-44. Langkjaer et al 2003 “Yeast genome duplication was followed by asynchronous differentiation of duplicated genes.” Nature 428:848-852. Wolfe and Shields 1997 “Molecular evidence for an ancient duplication of the entire yeast genome.” Nature 387:708-713.) Using this major evolutionary event, yeast can be divided into species that diverged from a common ancestor following the WGD event (termed “post-WGD yeast” herein) and species that diverged from the yeast lineage prior to the WGD event (termed “pre-WGD yeast” herein).

Accordingly, in one embodiment, the yeast microorganism may be selected from a post-WGD yeast genus, including but not limited to Saccharomyces and Candida. The favored post-WGD yeast species include: S. cerevisiae, S. uvarum, S. bayanus, S. paradoxus, S. castelli, and C. glabrata.

In another embodiment, the yeast microorganism may be selected from a pre-whole genome duplication (pre-WBD) yeast genus including but not limited to Saccharomyces, Kluyveromyces, Candida, Pichia, Debaryomyces, Hansenula, Pachysolen, Yarrowia and, Schizosaccharomyces. Representative pre-WGD yeast species include: S. kluyveri, K. thermotolerans, K. marxianus, K. waltii, K. lactis, C. tropicalis, P. pastoris, P. anomala, P. stipitis, P. kudriavzevii, D. hansenii, H. anomala, P. tannophilis, Y. lipolytica, and S. pombe.

A yeast microorganism may be either Crabtree-negative or Crabtree-positive. A yeast cell having a Crabtree-negative phenotype is any yeast cell that does not exhibit the Crabtree effect. The term “Crabtree-negative” refers to both naturally occurring and genetically modified organisms. Briefly, the Crabtree effect is defined as the inhibition of oxygen consumption by a microorganism when cultured under aerobic conditions due to the presence of a high concentration of glucose (e.g., 50 g-glucose L⁻¹). In other words, a yeast cell having a Crabtree-positive phenotype continues to ferment irrespective of oxygen availability due to the presence of glucose, while a yeast cell having a Crabtree-negative phenotype does not exhibit glucose mediated inhibition of oxygen consumption.

Accordingly, in one embodiment the yeast microorganism may be selected from yeast with a Crabtree-negative phenotype including but not limited to the following genera: Kluyveromyces, Pichia, Hansenula, and Candida. Crabtree-negative species include but are not limited to: K. lactis, K. marxianus, P. anomala, P. stipitis, P. kudriavzevii, H. anomala, and C. utilis.

In another embodiment, the yeast microorganism may be selected from a yeast with a Crabtree-positive phenotype, including but not limited to Saccharomyces, Kluyveromyces, Zygosaccharomyces, Debaryomyces, Pichia and Schizosaccharomyces. Crabtree-positive yeast species include but are not limited to: S. cerevisiae, S. uvarum, S. bayanus, S. paradoxus, S. castelli, S. kluyveri, K. thermotolerans, C. glabrata, Z. bailli, Z. rouxii, D. hansenii, P. pastorius, and S. pombe.

In one embodiment, a yeast microorganism is engineered to convert a carbon source, such as glucose, to pyruvate by glycolysis and the pyruvate is converted to isobutanol via an engineered isobutanol pathway (See, e.g., PCT/US2006/041602 and PCT/US2008/053514). Alternative pathways for the production of isobutanol have been described in International Patent Application No PCT/US2006/041602 and in Dickinson et al., Journal of Biological Chemistry 273: 25751-15756 (1998).

Accordingly, the engineered isobutanol pathway to convert pyruvate to isobutanol can be, but is not limited to, the following reactions:

1. 2 pyruvate→acetolactate+CO₂

2. acetolactate+NAD(P)H→2,3-dihydroxyisovalerate+NAD(P)⁺

3. 2,3-dihydroxyisovalerate→alpha-ketoisovalerate

4. alpha-ketoisovalerate→isobutyraldehyde+CO₂

5. isobutyraldehyde+NAD(P)H→isobutanol+NAD(P)⁺

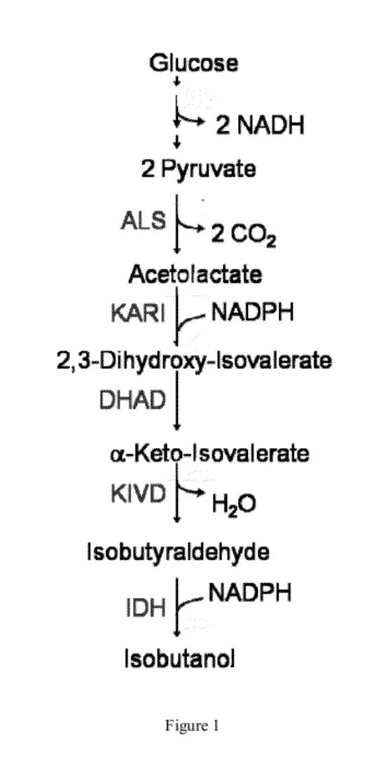

These reactions are carried out by the enzymes 1) Acetolactate Synthase (ALS), 2) Ketol-acid Reducto-Isomerase (KARI), 3) Dihydroxy-acid dehydratase (DHAD), 4) Keto-isovalerate decarboxylase (KIVD), and 5) an Isobutyraldehyde Dehydrogenase (IDH) (FIG. 1).

In another embodiment, the yeast microorganism is engineered to overexpress these enzymes. For example, ALS can be encoded by the alsS gene of B. subtilis, alsS of L. lactis, or the ilvK gene of K. pneumonia. For example, KARI can be encoded by the ilvC genes of E. coli, C. glutamicum, M. maripaludis, or Piromyces sp E2. For example, DHAD can be encoded by the ilvD genes of E. coli or C. glutamicum. KIVD can be encoded by the LlkivD gene of L. lactis. IDH can be encoded by ADH2, ADH6, or ADH7 of S. cerevisiae. Isobutyraldehyde dehydrogenase or IDH is defined as an alcohol dehydrogenase (ADH) that catalyzes the conversion of isobutyraldehyde to isobutanol.

The yeast microorganism of the invention may be engineered to have increased ability to convert pyruvate to isobutanol. In one embodiment, the yeast microorganism may be engineered to have increased ability to convert pyruvate to isobutyraldehyde. In another embodiment, the yeast microorganism may be engineered to have increased ability to convert pyruvate to keto-isovalerate. In another embodiment, the yeast microorganism may be engineered to have increased ability to convert pyruvate to 2,3-dihydroxyisovalerate. In another embodiment, the yeast microorganism may be engineered to have increased ability to convert pyruvate to acetolactate.

Furthermore, any of the genes encoding the foregoing enzymes (or any others mentioned herein (or any of the regulatory elements that control or modulate expression thereof)) may be optimized by genetic/protein engineering techniques, such as directed evolution or rational mutagenesis, which are known to those of ordinary skill in the art. Such action allows those of ordinary skill in the art to optimize the enzymes for expression and activity in yeast.

It is understood that various microorganisms can act as “sources” for genetic material encoding target enzymes suitable for use in a recombinant microorganism provided herein. For example, In addition, genes encoding these enzymes can be identified from other fungal and bacterial species and can be expressed for the modulation of this pathway. A variety of organisms could serve as sources for these enzymes, including, but not limited to, Saccharomyces spp., including S. cerevisiae and S. uvarum, Kluyveromyces spp., including K. thermotolerans, K. lactis, and K. marxianus, Pichia spp., Hansenula spp., including H. polymorpha, Candida spp., Trichosporon spp., Yamadazyma spp., including Y. stipitis, Schizosaccharomyces spp., including S. pombe, Cryptococcus spp., Aspergillus spp., or Neurospora spp. Sources of genes from anaerobic fungi include, but not limited to, Piromyces spp., Orpinomyces spp., or Neocallimastix spp. Sources of prokaryotic enzymes that are useful include, but not limited to, Escherichia. coli, Zymomonas mobilis, Staphylococcus aureus, Bacillus spp., Clostridium spp., Corynebacterium spp., Pseudomonas spp., Lactococcus spp., Enterobacter spp., and Salmonella spp.

### Methods in General

### Gene Expression

In another embodiment a method of producing a recombinant microorganism that converts a suitable carbon substrate to isobutanol is provided. The method includes transforming a microorganism with one or more recombinant polynucleotides encoding polypeptides that include but are not limited to, for example, ALS, KARI, DHAD, KIVD, IDH. Polynucleotides that encode enzymes useful for generating metabolites including homologs, variants, fragments, related fusion proteins, or functional equivalents thereof, are used in recombinant nucleic acid molecules that direct the expression of such polypeptides in appropriate host cells, such as bacterial or yeast cells. It is understood that the addition of sequences which do not alter the encoded activity of a polynucleotide, such as the addition of a non-functional or non-coding sequence, is a conservative variation of the basic nucleic acid. The “activity” of an enzyme is a measure of its ability to catalyze a reaction resulting in a metabolite, i.e., to “function”, and may be expressed as the rate at which the metabolite of the reaction is produced. For example, enzyme activity can be represented as the amount of metabolite produced per unit of time or per unit of enzyme (e.g., concentration or weight), or in terms of affinity or dissociation constants.

Those of skill in the art will recognize that, due to the degenerate nature of the genetic code, a variety of DNA compounds differing in their nucleotide sequences can be used to encode a given amino acid sequence of the disclosure. The native DNA sequence encoding the biosynthetic enzymes described herein are referenced herein merely to illustrate an embodiment of the disclosure, and the disclosure includes DNA compounds of any sequence that encode the amino acid sequences of the polypeptides and proteins of the enzymes utilized in the methods of the disclosure. In similar fashion, a polypeptide can typically tolerate one or more amino acid substitutions, deletions, and insertions in its amino acid sequence without loss or significant loss of a desired activity. The disclosure includes such polypeptides with alternate amino acid sequences, and the amino acid sequences encoded by the DNA sequences shown herein merely illustrate embodiments of the disclosure.

The disclosure provides nucleic acid molecules in the form of recombinant DNA expression vectors or plasmids, as described in more detail below, that encode one or more target enzymes. Generally, such vectors can either replicate in the cytosol of the host microorganism or integrate into the chromosomal DNA of the host microorganism. In either case, the vector can be a stable vector (i.e., the vector remains present over many cell divisions, even if only with selective pressure) or a transient vector (i.e., the vector is gradually lost by host microorganisms with increasing numbers of cell divisions). The disclosure provides DNA molecules in isolated (i.e., not pure, but existing in a preparation in an abundance and/or concentration not found in nature) and purified (i.e., substantially free of contaminating materials or substantially free of materials with which the corresponding DNA would be found in nature) forms.

Provided herein are methods for the expression of one or more of the biosynthetic genes involved in isobutanol biosynthesis and recombinant DNA expression vectors useful in the method. Thus, included within the scope of the disclosure are recombinant expression vectors that include such nucleic acids. The term expression vector refers to a nucleic acid that can be introduced into a host microorganism or cell-free transcription and translation system. An expression vector can be maintained permanently or transiently in a microorganism, whether as part of the chromosomal or other DNA in the microorganism or in any cellular compartment, such as a replicating vector in the cytosol. An expression vector also comprises a promoter that drives expression of an RNA, which typically is translated into a polypeptide in the microorganism or cell extract. For efficient translation of RNA into protein, the expression vector also typically contains a ribosome-binding site sequence positioned upstream of the start codon of the coding sequence of the gene to be expressed. Other elements, such as enhancers, secretion signal sequences, transcription termination sequences, and one or more marker genes by which host microorganisms containing the vector can be identified and/or selected, may also be present in an expression vector. Selectable markers, i.e., genes that confer antibiotic resistance or sensitivity, are used and confer a selectable phenotype on transformed cells when the cells are grown in an appropriate selective medium.

The various components of an expression vector can vary widely, depending on the intended use of the vector and the host cell(s) in which the vector is intended to replicate or drive expression. Expression vector components suitable for the expression of genes and maintenance of vectors in E. coli, yeast, Streptomyces, and other commonly used cells are widely known and commercially available. For example, suitable promoters for inclusion in the expression vectors of the disclosure include those that function in eukaryotic or prokaryotic host microorganisms. Promoters can comprise regulatory sequences that allow for regulation of expression relative to the growth of the host microorganism or that cause the expression of a gene to be turned on or off in response to a chemical or physical stimulus. For E. coli and certain other bacterial host cells, promoters derived from genes for biosynthetic enzymes, antibiotic-resistance conferring enzymes, and phage proteins can be used and include, for example, the galactose, lactose (lac), maltose, tryptophan (tip), beta-lactamase (bla), bacteriophage lambda PL, and T5 promoters. In addition, synthetic promoters, such as the tac promoter (U.S. Pat. No. 4,551,433), can also be used. For E. coli expression vectors, it is useful to include an E. coli origin of replication, such as from pUC, p1P, p1, and pBR.

Thus, recombinant expression vectors contain at least one expression system, which, in turn, is composed of at least a portion of a biosynthetic gene coding sequences operably linked to a promoter and optionally termination sequences that operate to effect expression of the coding sequence in compatible host cells. The host cells are modified by transformation with the recombinant DNA expression vectors of the disclosure to contain the expression system sequences either as extrachromosomal elements or integrated into the chromosome.

Moreover, methods for expressing a polypeptide from a nucleic acid molecule that are specific to yeast microorganisms are well known. For example, nucleic acid constructs that are used for the expression of heterologous polypeptides within Kluyveromyces and Saccharomyces are well known (see, e.g., U.S. Pat. Nos. 4,859,596 and 4,943,529, each of which is incorporated by reference herein in its entirety for Kluyveromyces and, e.g., Gellissen et al., Gene 190(1):87-97 (1997) for Saccharomyces. Yeast plasmids have a selectable marker and an origin of replication, also known as Autonomously Replicating Sequences (ARS). In addition certain plasmids may also contain a centromeric sequence. These centromeric plasmids are generally a single or low copy plasmid. Plasmids without a centromeric sequence and utilizing either a 2 micron (S. cerevisiae) or 1.6 micron (K lactis) replication origin are high copy plasmids. The selectable marker can be either prototrophic, such as HIS3, TRP1, LEU2, URA3 or ADE2, or antibiotic resistance, such as, bar, ble, hph, or kan.

A nucleic acid of the disclosure can be amplified using cDNA, mRNA or alternatively, genomic DNA, as a template and appropriate oligonucleotide primers according to standard PCR amplification techniques and those procedures described in the Examples section below. The nucleic acid so amplified can be cloned into an appropriate vector and characterized by DNA sequence analysis. Furthermore, oligonucleotides corresponding to nucleotide sequences can be prepared by standard synthetic techniques, e.g., using an automated DNA synthesizer.

It is also understood that an isolated nucleic acid molecule encoding a polypeptide homologous to the enzymes described herein can be created by introducing one or more nucleotide substitutions, additions or deletions into the nucleotide sequence encoding the particular polypeptide, such that one or more amino acid substitutions, additions or deletions are introduced into the encoded protein. Mutations can be introduced into the polynucleotide by standard techniques, such as site-directed mutagenesis and PCR-mediated mutagenesis. In contrast to those positions where it may be desirable to make a non-conservative amino acid substitutions (see above), in some positions it is preferable to make conservative amino acid substitutions. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

### Identification of Genes in a Host Microorganism

Any method can be used to identify genes that encode for enzymes with a specific activity. Generally, homologous or analogous genes with similar activity can be identified by functional, structural, and/or genetic analysis. In most cases, homologous or analogous genes with similar activity will have functional, structural, or genetic similarities. Techniques known to those skilled in the art may be suitable to identify homologous genes and homologous enzymes. Generally, analogous genes and/or analogous enzymes can be identified by functional analysis and will have functional similarities. Techniques known to those skilled in the art may be suitable to identify analogous genes and analogous enzymes. For example, to identify homologous or analogous genes, proteins, or enzymes, techniques may include, but not limited to, cloning a gene by PCR using primers based on a published sequence of a gene/enzyme or by degenerate PCR using degenerate primers designed to amplify a conserved region among a gene. Further, one skilled in the art can use techniques to identify homologous or analogous genes, proteins, or enzymes with functional homology or similarity. Techniques include examining a cell or cell culture for the catalytic activity of an enzyme through in vitro enzyme assays for said activity, then isolating the enzyme with said activity through purification, determining the protein sequence of the enzyme through techniques such as Edman degradation, design of PCR primers to the likely nucleic acid sequence, amplification of said DNA sequence through PCR, and cloning of said nucleic acid sequence. To identify homologous or analogous genes with similar activity, techniques also include comparison of data concerning a candidate gene or enzyme with databases such as BRENDA, KEGG, or MetaCYC. The candidate gene or enzyme may be identified within the above mentioned databases in accordance with the teachings herein. Furthermore, enzymatic activity can be determined phenotypically. For example, ethanol production under fermentative conditions can be assessed. A lack of ethanol production may be indicative of a microorganism lacking an alcohol dehydrogenase.

### Genetic Insertions and Deletions

Any method can be used to introduce a nucleic acid molecule into the chromosomal DNA of a microorganism and many such methods are well known. For example, lithium acetate transformation and electroporation are common methods for introducing nucleic acid into yeast microorganisms. See, e.g., Gietz et al., Nucleic Acids Res. 27:69-74 (1992); Ito et al., J. Bacterol. 153:163-168 (1983); and Becker and Guarente, Methods in Enzymology 194:182-187 (1991).

In an embodiment, the integration of a gene of interest into a DNA fragment or target gene of a yeast microorganism occurs according to the principle of homologous recombination. According to this embodiment, an integration cassette containing a module comprising at least one yeast marker gene and/or the gene to be integrated (internal module) is flanked on either side by DNA fragments homologous to those of the ends of the targeted integration site (recombinogenic sequences). After transforming the yeast with the cassette by appropriate methods, a homologous recombination between the recombinogenic sequences may result in the internal module replacing the chromosomal region in between the two sites of the genome corresponding to the recombinogenic sequences of the integration cassette. (Orr-Weaver et al., PNAS USA 78:6354-6358 (1981))

In an embodiment, the integration cassette for integration of a gene of interest into a yeast microorganism includes the heterologous gene under the control of an appropriate promoter and terminator together with the selectable marker flanked by recombinogenic sequences for integration of a heterologous gene into the yeast chromosome. In an embodiment, the heterologous gene includes an appropriate native gene desired to increase the copy number of a native gene(s). The selectable marker gene can be any marker gene used in yeast, including but not limited to, HIS3, TRP1, LEU2, URA3, bar, ble, hph, and kan. The recombinogenic sequences can be chosen at will, depending on the desired integration site suitable for the desired application.

Additionally, in an embodiment pertaining to yeast microorganisms, certain introduced marker genes are removed from the genome using techniques well known to those skilled in the art. For example, URA3 marker loss can be obtained by plating URA3 containing cells in FOA (5-fluoro-orotic acid) containing medium and selecting for FOA resistant colonies (Boeke, J. et al, 1984, Mol. Gen. Genet, 197, 345-47).

Integration of all the genes of a metabolic pathway that lead to a product into the genome of the production strain eliminates the need of a plasmid expression system, as the enzymes are produced from the chromosome. The integration of pathway genes avoids loss of productivity over time due to plasmid loss. This is important for long fermentation times and for fermentations in large scale where the seed train is long and the production strain has to go through many doublings from the first inoculation to the end of the large scale fermentation.

Integrated genes are maintained in the strain without selection. This allows the construction of production strains that are free of marker genes which are commonly used for maintenance of plasmids. Production strains with integrated pathway genes can contain minimal amounts of foreign DNA since there are no origins of replication and other non coding DNA necessary that have to be in plasmid based systems. The biocatalyst with integrated pathway genes improves the yield of a production process because it avoids energy and carbon requiring processes. These processes are the replication of many copies of plasmids and the production of non-pathway active proteins like marker proteins in the production strain.

The expression of pathway genes on multi-copy plasmids can lead to overexpression phenotypes for certain genes. These phenotypes can be growth retardation, inclusion bodies, and cell death. Therefore the expression levels of genes on multi copy plasmids has to be controlled effectively by using inducible expression systems, optimizing the time of induction of said expression system, and optimizing the amount of inducer provided. The time of induction has to be correlated to the growth phase of the biocatalyst, which can be followed by measuring of optical density in the fermentation broth. A biocatalyst that has all pathway genes integrated on its chromosome is far more likely to allow constitutive expression since the lower number of gene copies avoids over expression phenotypes.

### Overexpression of Heterologous Genes

Methods for overexpressing a polypeptide from a native or heterologous nucleic acid molecule are well known. Such methods include, without limitation, constructing a nucleic acid sequence such that a regulatory element promotes the expression of a nucleic acid sequence that encodes the desired polypeptide. Typically, regulatory elements are DNA sequences that regulate the expression of other DNA sequences at the level of transcription. Thus, regulatory elements include, without limitation, promoters, enhancers, and the like. For example, the exogenous genes can be under the control of an inducible promoter or a constitutive promoter. Moreover, methods for expressing a polypeptide from an exogenous nucleic acid molecule in yeast are well known. For example, nucleic acid constructs that are used for the expression of exogenous polypeptides within Kluyveromyces and Saccharomyces are well known (see, e.g., U.S. Pat. Nos. 4,859,596 and 4,943,529, for Kluyveromyces and, e.g., Gellissen et al., Gene 190(1):87-97 (1997) for Saccharomyces). Yeast plasmids have a selectable marker and an origin of replication. In addition certain plasmids may also contain a centromeric sequence. These centromeric plasmids are generally a single or low copy plasmid. Plasmids without a centromeric sequence and utilizing either a 2 micron (S. cerevisiae) or 1.6 micron (K lactis) replication origin are high copy plasmids. The selectable marker can be either prototrophic, such as HIS3, TRP1, LEU2, URA3 or ADE2, or antibiotic resistance, such as, bar, ble, hph, or kan.

In another embodiment, heterologous control elements can be used to activate or repress expression of endogenous genes. Additionally, when expression is to be repressed or eliminated, the gene for the relevant enzyme, protein or RNA can be eliminated by known deletion techniques.

As described herein, any yeast within the scope of the disclosure can be identified by selection techniques specific to the particular enzyme being expressed, over-expressed or repressed. Methods of identifying the strains with the desired phenotype are well known to those skilled in the art. Such methods include, without limitation, PCR, RT-PCR, and nucleic acid hybridization techniques such as Northern and Southern analysis, altered growth capabilities on a particular substrate or in the presence of a particular substrate, a chemical compound, a selection agent and the like. In some cases, immunohistochemistry and biochemical techniques can be used to determine if a cell contains a particular nucleic acid by detecting the expression of the encoded polypeptide. For example, an antibody having specificity for an encoded enzyme can be used to determine whether or not a particular yeast cell contains that encoded enzyme. Further, biochemical techniques can be used to determine if a cell contains a particular nucleic acid molecule encoding an enzymatic polypeptide by detecting a product produced as a result of the expression of the enzymatic polypeptide. For example, transforming a cell with a vector encoding acetolactate synthase and detecting increased cytosolic acetolactate concentrations compared to a cell without the vector indicates that the vector is both present and that the gene product is active. Methods for detecting specific enzymatic activities or the presence of particular products are well known to those skilled in the art. For example, the presence of acetolactate can be determined as described by Hugenholtz and Starrenburg, Appl. Microbiol. Biotechnol. 38:17-22 (1992).

### Reduction of Enzymatic Activity

Host microorganisms within the scope of the invention may have reduced enzymatic activity such as reduced alcohol dehydrogenase activity. The term “reduced” as used herein with respect to a particular enzymatic activity refers to a lower level of enzymatic activity than that measured in a comparable host cell of the same species. Thus, host cells lacking alcohol dehydrogenase activity are considered to have reduced alcohol dehydrogenase activity since most, if not all, comparable host cells of the same species have at least some alcohol dehydrogenase activity. Such reduced enzymatic activities can be the result of lower enzyme expression level, lower specific activity of an enzyme, or a combination thereof. Many different methods can be used to make host cells having reduced enzymatic activity. For example, a host cell can be engineered to have a disrupted enzyme-encoding locus using common mutagenesis or knock-out technology. See, e.g., Methods in Yeast Genetics (1997 edition), Adams, Gottschling, Kaiser, and Stems, Cold Spring Harbor Press (1998), Datsenko and Wanner, Proc. Natl. Acad. Sci. USA 97, 6640-6645, 2000.

In addition, certain point-mutation(s) can be introduced which results in an enzyme with reduced activity.

Alternatively, antisense technology can be used to reduce enzymatic activity. For example, host cells can be engineered to contain a cDNA that encodes an antisense molecule that prevents an enzyme from being made. The term “antisense molecule” as used herein encompasses any nucleic acid molecule that contains sequences that correspond to the coding strand of an endogenous polypeptide. An antisense molecule also can have flanking sequences (e.g., regulatory sequences). Thus antisense molecules can be ribozymes or antisense oligonucleotides. A ribozyme can have any general structure including, without limitation, hairpin, hammerhead, or axhead structures, provided the molecule cleaves RNA.

Host cells having a reduced enzymatic activity can be identified using many methods. For example, host cells having reduced alcohol dehydrogenase activity can be easily identified using common methods, which may include, for example, measuring ethanol formation via gas chromatography.

### Increase of Enzymatic Activity

Host microorganisms of the invention may be further engineered to have increased activity of enzymes. The term “increased” as used herein with respect to a particular enzymatic activity refers to a higher level of enzymatic activity than that measured in a comparable yeast cell of the same species. For example, overexpression of a specific enzyme can lead to an increased level of activity in the cells for that enzyme. Increased activities for enzymes involved in glycolysis or the isobutanol pathway would result in increased productivity and yield of isobutanol.

Methods to increase enzymatic activity are known to those skilled in the art. Such techniques may include increasing the expression of the enzyme by increasing plasmid copy number and/or use of a stronger promoter and/or use of activating riboswitches, introduction of mutations to relieve negative regulation of the enzyme, introduction of specific mutations to increase specific activity and/or decrease the Km for the substrate, or by directed evolution. See, e.g., Methods in Molecular Biology (vol. 231), ed. Arnold and Georgiou, Humana Press (2003).

### Microorganism in Detail

Microorganism Characterized by Production of Isobutanol from Pyruvate Via an Isobutanol Pathway Expressed in the Mitochondria at High Yield

For a biocatalyst to produce isobutanol most economically, it is desired to produce a high yield. Preferably, the only product produced is isobutanol. Extra products lead to a reduction in product yield and an increase in capital and operating costs, particularly if the extra products have little or no value. Extra products also require additional capital and operating costs to separate these products from isobutanol.

The microorganism may convert one or more carbon sources derived from biomass into isobutanol with a yield of greater than 5% of theoretical. In one embodiment, the yield is greater than 10%. In one embodiment, the yield is greater than 50% of theoretical.

In one embodiment, the yield is greater than 60% of theoretical. In another embodiment, the yield is greater than 70% of theoretical. In yet another embodiment, the yield is greater than 80% of theoretical. In yet another embodiment, the yield is greater than 85% of theoretical. In yet another embodiment, the yield is greater than 90% of theoretical. In yet another embodiment, the yield is greater than 95% of theoretical. In still another embodiment, the yield is greater than 97.5% of theoretical.

More specifically, the microorganism converts glucose, which can be derived from biomass into isobutanol with a yield of greater than 5% of theoretical. In one embodiment, the yield is greater than 10% of theoretical. In one embodiment, the yield is greater than 50% of theoretical. In one embodiment the yield is greater than 60% of theoretical. In another embodiment, the yield is greater than 70% of theoretical. In yet another embodiment, the yield is greater than 80% of theoretical. In yet another embodiment, the yield is greater than 85% of theoretical. In yet another embodiment the yield is greater than 90% of theoretical. In yet another embodiment, the yield is greater than 95% of theoretical. In still another embodiment, the yield is greater than 97.5% of theoretical

Microorganism Characterized by Production of Isobutanol from Pyruvate Via an Isobutanol Pathway Expressed in the Mitochondria of a PDC-Minus Yeast

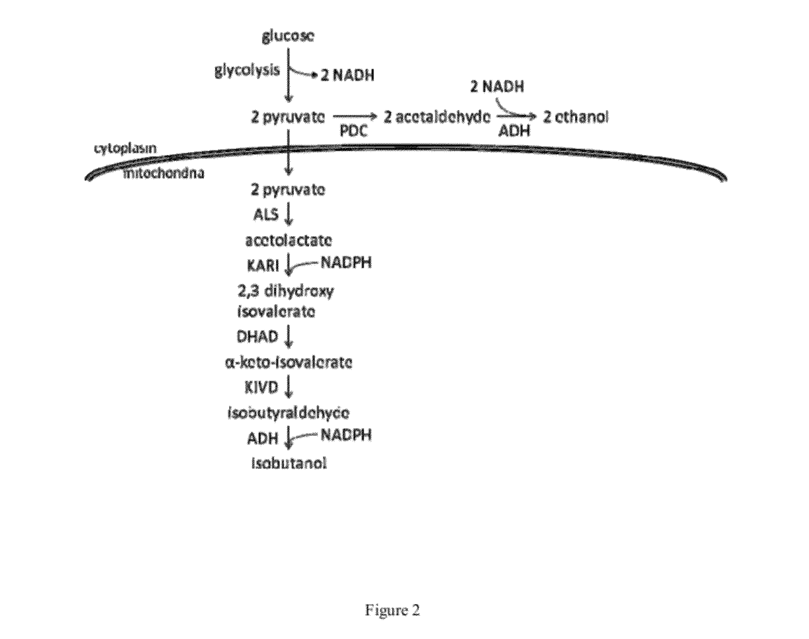

In yeast, the conversion of pyruvate to acetaldehyde is a major drain on the pyruvate pool (FIG. 2), and, hence, a major source of competition with the isobutanol pathway. This reaction is catalyzed by the pyruvate decarboxylase (PDC) enzyme. Reduction of this enzymatic activity in the yeast microorganism results in an increased availability of pyruvate and reducing equivalents to the isobutanol pathway and may improve isobutanol production and yield in a yeast microorganism that expresses a pyruvate-dependent isobutanol pathway.

Reduction of PDC activity can be accomplished by 1) mutation or deletion of a positive transcriptional regulator for the structural genes encoding for PDC or 2) mutation or deletion of all PDC encoding genes in a given organism. For example, in S. cerevisiae, the PDC2 gene, which encodes for a positive transcriptional regulator of PDC1, 5, 6 genes can be deleted; a S. cerevisiae in which the PDC2 gene is deleted is reported to have only ˜10% of wildtype PDC activity (Hohmann, Mol Gen Genet, 241:657-666 (1993)). Alternatively, for example, all structural genes for PDC (e.g. in S. cerevisiae, PDC1, PDC5, and PDC6, or in K. marxianus, PDC1) are deleted.

In addition to reduced ability to convert pyruvate to acetaldehyde, the yeast microorganism is engineered to have increased ability to convert ketoisovalerate to isobutyraldehyde. In many yeast microorganisms, both of these reactions can be catalyzed by the enzyme, pyruvate decarboxylase. For example, the ability to convert pyruvate to acetaldehyde is reduced as described above and the ketoisovalerate to isobutyraldehyde conversion is increased by introducing an enzyme that has a higher specificity to ketoisovalerate, such as the KIVD enzyme from L. lactis. Specifically, the microorganism has sufficiently low ability to convert pyruvate to acetaldehyde and sufficiently high ability to convert ketoisovalerate to isobutyraldehyde to result in an isobutanol yield of greater than 50%, 60%, 70%, 80%, 90%, 95%, 97.5% of theoretical.

Crabtree-positive yeast strains such as Saccharomyces.cerevisiae strain that contains disruptions in all three of the PDC alleles no longer produce ethanol by fermentation. However, a downstream product of the reaction catalyzed by PDC, acetyl-CoA, is needed for anabolic production of necessary molecules. Therefore, the Pdc-mutant is unable to grow solely on glucose, and requires a two-carbon carbon source, either ethanol or acetate, to synthesize acetyl-CoA. (Flikweert M T, de Swaaf M, van Dijken J P, Pronk J T. FEMS Microbiol Lett. 1999 May 1; 174(1):73-9. PMID:10234824 and van Maris A J, Geertman J M, Vermeulen A, Groothuizen M K, Winkler A A, Piper M D, van Dijken J P, Pronk J T. Appl Environ Microbiol. 2004 January; 70(1):159-66. PMID: 14711638).

Thus, in an embodiment, such a Crabtree-positive yeast strain may be evolved to generate variants of the PDC mutant that do not have the requirement for a two-carbon molecule and has a growth rate similar to wild-type on glucose. Any method, including chemostat evolution or serial dilution may be utilized to generate variants of strains with deletion of three PDC loci that can grow on glucose as the sole carbon source at a rate similar to wild type.

Most of the enzymatic activities that are needed for the metabolic conversion of pyruvate to isobutanol are present in yeast. ALS, KARI and DHAD activities are present as part of the branched chain amino acid biosynthetic pathway. These three enzymes are localized in the yeast mitochondria. Ketoacid decarboxylase (KIVD) activity is present in the yeast cytosol. The native S. cerevisiae enzymes catalyzing this conversion are Pdc1p, Pdc5p, and Pdc1p, Aro10p and Thi3p. THI3 is annotated as coding for an -ketoisocaproate decarboxylase and its gene product may have a role in catabolism of amino acids to long-chain and complex alcohols. It was shown that Thi3p is mainly responsible for the decarboxylation of -ketoisocaproate to isoamyl alcohol (Dickinson J R, et al. The Journal of Biological Chemistry 1997, 278:8028-8034). Deletion of THI3 did not have an effect on isobutanol production from valine (Dickinson, J R, et al. The Journal of Biological Chemistry 1998, 273:25751-25756). THI3 is required for expression of enzymes involved in thiamine biosynthesis. Aro10p is a phenylpyruvate decarboxylase, catalyzing the decarboxylation of phenylpyruvate to phenylacetaldehyde, which is the first specific step in the Ehrlich pathway. Aro10p was shown to have activity with ketoisovalerate when produced in E. coli (WO 2008/098227). The overexpression of ARO10 in S. cerevisiae indicated the involvement of posttranscriptional regulation and/or a second protein in the Aro10p dependent broad substrate specificity decarboxylase activity (Vuralhan, Z., et al. 2005, Appl. and Environ. Microbiol. 71: 3276-3284). Isobutanol production from ketoisovalerate in S. cerevisiae is catalyzed by any of the three PDC enzymes (Dickinson, J R, et al. 1998, J. Biol. Chem. 273: 25751-25756). The last step of the isobutanol pathway is catalyzed by an isobutyraldehyde dehydrogenase. In yeast there are several enzymes that potentially catalyze this reaction. Adh5p, Adh6p, and Adh7p are NADPH dependent and Adh1p, Adh2p, Adh3p, and Adh4p are NADH dependent. Adh1p, Adh2p, Adh5p, Adh6p, and Adh7p are cytosolic and Adh3p, and Adh4 are mitochondrial. An assessment of substrate specificity of Adh1p, Adh2p, Adh6p, and Adh7p showed that Adh7p shows the highest activity with isobutyraldehyde. Adh3p is involved in the acetaldehyde ethanol shuttle that transfers mitochondrial NADH into the cytosol under anaerobic conditions. Adh1-5proteins are all involved in ethanol metabolism. Sfa1p is localized in the cytosol as well as in the mitochondria. Sfa1p is involved in the formation of long chain and complex alcohols. It is a bifunctional enzyme which also reduces hydroxymethylfurfural using the cofactor NADH. This indicates that all ADHs that are expressed in the yeast mitochondria are NADH-dependent.

For the entire isobutanol pathway to be expressed in the yeast mitochondria KIVD has to be expressed in this compartment. All other activities are already natively expressed in the mitochondria. However to insure sufficient capacity of the pathway and to avoid the down regulation of the native expression levels of the pathway enzymes all or some of the pathway enzymes can be overexpressed. The overexpression of the enzymes that are already natively targeted to the mitochondria can be done by expressing the genes under the control of constitutive yeast promoters like P_{ADH}, or P_{PDC1}. Expression levels can be adjusted by strength of promoter or copy number. For the expression of ARO10 or THI3, which are not natively localized to the mitochondria a yeast mitochondrial targeting sequence (MTS) has to be added to the coding sequence of the genes of interest. Several MTS have been described in the literature. Examples are the Cox4p MTS and the MTS of ScHMI1p. The MTS of proteins can be predicted using software that detects the typical arrangement of charged and hydrophobic residues that can be found in proteins targeted to the mitochondria. These programs also predict the likely localization of heterologous proteins in a yeast host cell. Examples of such programs are mitoprot and psort.

Addition of yeast mitochondrial targeting sequences is likely necessary for the mitochondrial expression of homologs of the isobutanol pathway enzymes that are not native to the host organism. These leader sequences are 10-80 amino acids long and are usually on the N-terminus of the proteins although one native example for a C-terminal leader sequence has been reported. Also, N-terminal leader sequences have been shown to be functional if attached to the C-terminus of a protein. In addition to native leader sequences artificial leader sequences have been constructed that were functional but did not reach the efficiency of their natural counterparts. Leader sequences have a secondary structure that leads to an alpha helix that is positively charged on one side and hydrophobic on the opposite side. The charged as well as the hydrophobic side of the helix facilitate the transfer of the protein through the two mitochondrial membranes into the mitochondrial matrix. Once the leader sequence is inside the matrix it is removed from the protein by the mitochondrial protein peptidase (MPP) activity. The specificity of the protease is such that a basic amino acid, in most cases arginine is found at the −2 position of the MPP cleavage site. In addition of this recognition of sequence close to its cleavage site MPP also recognizes the amino terminal targeting sequence. In some cases the leader sequence is cut twice. After the cleavage by MPP the protein is cut again by mitochondrial intermediate peptidase (MIP) or the terminal amino acid after the MPP cut is removed by ICP55. N-terminal as well as C-terminal extensions of a protein sequence can impair enzymatic activity as well as protein folding. Depending on the properties of each enzyme a decision can be made on whether a 3′ or a 5′ attachment of the leader sequence is more likely to render a correctly folded and active protein.

Overexpression of isobutanol pathway enzymes in the mitochondria of a yeast cell can cause overexpression phenotypes ranging from growth retardation to non-viability. To avoid these phenotypes and to enable fast cell growth prior to the isobutanol production phase the expression of the pathway genes can be limited and controlled. Limitation of the expression level can be accomplished by the use of weak promoters and by reduction of the copy number of the expression cassette. However these measures can limit the productivity of the strain if the pathway enzyme activity is limiting the isobutanol productivity of the strain. Control of expression can be accomplished by use of inducible promoters. Repression of the expression of one or more pathway genes during growth allows for fast production of biomass. Induction of the pathway enzymes allows for high productivity during isobutanol production. Inducible promoters that are useful in yeast comprise P_{MET3}, and P_{MET17}, which are repressed by methionine, P_{CUP} which is induced by copper, P_{PDC1}, and P_{ADH1} which are induced by glucose. Given are the gene and promoter names for Saccharomyces cerevisiae. To insure proper regulation native promoters should be used in each of the different host organisms.

In addition to or separate from the deletion or reduction in the activity of PDC, the recombinant microorganisms of the present invention may further include the deletion or reduction of the activity of additional enzymes that (a) directly consume a precursor of the product, e.g. an isobutanol precursor, (b) indirectly consume a precursor of the product, e.g. of isobutanol, or (c) repress the expression or function of a pathway that supplies a precursor of the product, e.g. of isobutanol. These enzymes include glycerol-3-phosphate dehydrogenase (encoded, e.g. by GPD1 or GPD2 of S. cerevisiae) an alcohol dehydrogenase (encoded, e.g., by adhE of E. coli or ADH1, ADH2, ADH3, ADH4, ADH5, ADH6, or ADH7 of S. cerevisiae), 2-isopropylmalate synthase (encoded, e.g. by LEU4 or LEU9 of S. cerevisiae), valine transaminase (encoded, e.g. by BAT1 or BAT2 of S. cerevisiae), Threonine deaminase (encoded, e.g. by ilvA of E. coli or CHA1 or ILV1 of S. cerevisiae), or any combination thereof, to increase the availability of pyruvate or reduce enzymes that compete for a metabolite in a desired biosynthetic pathway.

### Microorganism Characterized by Balancing Cofactor Usage

The ideal production microorganism produces a desirable product at close to theoretical yield. For example the ideal isobutanol producing organism produces isobutanol according to the following equation:

glucose→isobutanol+2CO₂+H₂O

Accordingly, 66% of the glucose carbon results in isobutanol, while 33% is lost as CO₂. In exemplary metabolic pathways for the conversion of pyruvate to isobutanol described by Atsumi et al. (Atsumi et al., Nature, 2008 Jan. 3; 451(7174):86-9; International Patent Application No PCT/US2008/053514, which is herein incorporated by reference) two of the five enzymes used to convert pyruvate into isobutanol according to the metabolic pathway outlined in FIG. 1 require the reduced cofactor nicotinamide adenine dinucleotide phosphate (NADPH). NADPH is produced only sparingly by the cell—the reduced cofactor nicotinamide adenine dinucleotide (NADH) is the preferred equivalent. Respiration is required to produce NADPH in the large quantities required to support high-level production of isobutanol.

Even if competing pathways can be eliminated or reduced in activity by metabolic engineering, yield is limited to about 83% of theoretical. Carbon loss to carbon dioxide (CO₂) remains the main limitation on yield in the aforementioned metabolic pathway for the production of isobutanol.

In a metabolically engineered cell utilizing the aforementioned metabolic pathway the production of isobutanol from glucose results in an imbalance between the cofactors reduced during glycolysis and the cofactors oxidized during the conversion of pyruvate to isobutanol. While glycolysis produces 2 NADH, the isobutanol pathway consumes 2 NADPH. This leads to a deficit of 2 NADPH and overproduction of 2 NADH per isobutanol molecule produced, a state described henceforth as cofactor imbalance.

The terms “NADH dependent” or “NADPH dependent”, refer to the property of an enzyme to preferentially use either of the redox cofactors. A NADH dependent enzyme has a higher catalytic efficiency (k_{cat}/K_{M}) with the cofactor NADH than with the cofactor NADPH as determined by in vitro enzyme activity assays.

The terms “cofactor balance” or “balanced with respect to cofactor usage” refer to a recombinant microorganism comprising a metabolic pathway converting a carbon source to a fermentation product and a modification that leads to the regeneration of all redox cofactors within the recombinant microorganism producing said fermentation product from a carbon source and wherein the re-oxidation or re-reduction of said redox cofactors does not require the pentose phosphate pathway, the TCA cycle or the generation of additional fermentation products.

Stated another way, the terms “cofactor balance” or “balanced with respect to cofactor usage” can refer to an advantageous modification that leads to the regeneration of all redox cofactors within the recombinant microorganism producing a fermentation product from a carbon source and wherein said re-oxidation or re-reduction of all redox cofactors does not require the production of byproducts or co-products.

Stated another way, the terms “cofactor balance” or “balanced with respect to cofactor usage” can refer to an advantageous modification that leads to the regeneration of all redox cofactors within the recombinant microorganism producing a fermentation product from a carbon source under anaerobic conditions and wherein the production of additional fermentation products is not required for re-oxidation or re-reduction of redox cofactors.

Stated another way, the terms “cofactor balance” or “balanced with respect to cofactor usage” can refer to an advantageous modification that leads to the regeneration of all redox cofactors within the recombinant microorganism producing a fermentation product from a carbon source and wherein said modification increases production of said fermentation product under anaerobic conditions compared to the parental or wild-type microorganism and wherein additional fermentation products are not required for the regeneration of said redox cofactors.

The terms “partial cofactor balance” or “partially balanced with respect to cofactor usage” refer to a recombinant microorganism comprising a metabolic pathway converting a carbon source to a fermentation product and a modification that leads to the regeneration of redox cofactors within the recombinant microorganism producing said fermentation product from a carbon source and wherein the re-oxidation or re-reduction of said redox cofactors shows a reduced requirement of the pentose phosphate pathway, the TCA cycle or the generation of additional fermentation products when compared to the same recombinant microorganism without said modification.

Stated another way, the terms “partial cofactor balance” or “partially balanced with respect to cofactor usage” can refer to an advantageous modification that leads to the regeneration of redox cofactors within the recombinant microorganism producing a fermentation product from a carbon source and wherein said re-oxidation or re-reduction of all redox cofactors produces less byproducts or co-products when compared to the same recombinant microorganism without said advantageous modification.

The cell has several options for resolving a cofactor imbalance. One is to change the relative fluxes going from glucose through glycolysis and through the pentose phosphate pathway (PPP). For each glucose molecule metabolized through the PPP, 2 NADPH are generated in addition to the 2 NADH that are generated through glycolysis (a total of 4 reducing equivalents). Therefore, use of the PPP results in the generation of excess reducing equivalents since only two reducing equivalents are consumed during the production of isobutanol. Flux through the PPP results in the loss of one additional molecule of CO₂ per molecule of glucose consumed, which limits the yield of isobutanol that can be achieved. The production of excess reducing equivalents leads to the production of reduced byproducts or it necessitates respiration.

Another way the cell can generate NADPH is via the TCA cycle. Flux through the TCA cycle results in carbon loss through CO₂ and in production of NADH in addition to the NADPH required for the isobutanol pathway. The excess NADH would have to be utilized for energy production through respiration or for byproduct formation. Thus any flux through the TCA cycle will reduce the yield of the isobutanol production and it will require oxygen.

In addition to this basic cofactor imbalance, in yeast, NAD(P)H is not able to efficiently cross the mitochondrial membrane. Thus, to make available reducing equivalents for the isobutanol pathway in the mitochondria, an NADH shuttle (reviewed in Bakker et al., 2001, FEMS Microbiology Reviews 25:15-37) could be used or under aerobic conditions, the TCA cycle can be used to generate NADH in the mitochondria. An example of an NADH shuttle is the ethanol/acetaldehyde shuttle. An alcohol dehydrogenase (ADH) in the cytosol can oxidize an NADH to NAD+ while converting an acetaldehyde to ethanol. As both ethanol and acetaldehyde freely transverse membranes, generated ethanol can be used to reduce an NAD+ to NADH in the mitochondria by a mitochondrially localized ADH and generate acetaldehyde. In this fashion, a cytosolic NADH can be shuttled into the mitochondria. The use of the TCA cycle or the NADH shuttles would still result in a cofactor imbalance in the mitochondria as the isobutanol pathway consumes NADPH.

An economically competitive isobutanol process requires a high yield from a carbon source. Lower yield means that more feedstock is required to produce the same amount of isobutanol. Feedstock cost is the major component of the overall operating cost, regardless of the nature of the feedstock and its current market price. From an economical perspective, this is important because the cost of isobutanol is dependent on the cost of the biomass-derived sugars. An increase in feedstock cost results in an increase in isobutanol cost.

Thus, in one embodiment of the invention, a recombinant microorganism comprising a modification of the metabolic pathway for the production of a fermentation product wherein said modification balances the cofactor usage of the recombinant microorganism producing said fermentation product from a carbon source is provided.

In a specific aspect, a microorganism is provided in which cofactor usage is balanced during the production of isobutanol, in this case, production of isobutanol from pyruvate utilizes the same cofactor that is produced during glycolysis and shuttled into the mitochondria.

In another embodiment, a microorganism is provided in which cofactor usage is balanced during the production of a fermentation product and the microorganism produces the fermentation product at a higher yield compared to a modified microorganism in which the cofactor usage in not balanced.

In a specific aspect, a microorganism is provided in which cofactor usage is balanced during the production of isobutanol and the microorganism produces isobutanol at a higher yield compared to a modified microorganism in which the cofactor usage in not balanced.

In yet another embodiment, a modified microorganism in which cofactor usage is balanced during the production of a fermentation product may allow the microorganism to produce said fermentation product under anaerobic conditions, conditions under which a modified microorganism in which the cofactor usage in not balanced during production of a fermentation product may not be able to produce a fermentation product.

In a specific aspect, a modified microorganism in which cofactor usage is balanced during the production of isobutanol may allow the microorganism to produce said isobutanol under anaerobic conditions, conditions under which a modified microorganism in which the cofactor usage is not balanced during production of isobutanol may not be able to produce isobutanol.

One compound to be produced by the recombinant microorganism according to the present invention is isobutanol. However, the present invention is not limited to isobutanol. The invention may be applicable to any metabolic pathway that is imbalanced with respect to cofactor usage. One skilled in the art is able to identify pathways that are imbalanced with respect to cofactor usage and apply this invention to provide recombinant microorganisms in which the same pathway is balanced with respect to cofactor usage. One skilled in the art will recognize that the identified pathways may be of longer or shorter length, contain more or fewer genes or proteins, and require more or fewer cofactors than the exemplary isobutanol pathway. Further, one skilled in the art will recognize that in certain embodiments, such as a recombinant microbial host that produces an excess of NADPH, certain embodiments of the present invention may be adapted to convert NADPH to NADH.

Microorganism Characterized by Providing Cofactor Balance Via KARI and ADH that are Able to Utilize NADH.

As detailed above, production of isobutanol from glucose using the aforementioned pathway in the mitochondria results in cofactor imbalance as 2 NADHs are generated in the cytosol via glycolysis and 2 NADPHs are consumed in the mitochondria by the isobutanol pathway. Yeast cells are able to transfer the reducing equivalents from the cytosol to the mitochondria via an NADH shuttle. This results in an NADH in the mitochondria, which would still result in a cofactor imbalance with the isobutanol pathway. This imbalance can be resolved by the use of a mitochondrial isobutanol pathway that consumes NADH instead of NADPH. For the mitochondrial isobutanol pathway to consume NADH, the NADPH dependent enzymes, KARI and ADH can be replaced by either an NADH dependent homolog or an enzyme that has been engineered to use NADH.

The NADH shuttles that are available to transfer cytosolic NADH into the mitochondria have a limited capacity and might limit the productivity of a yeast isobutanol production strain if said strain produces all isobutanol pathway enzymes in the mitochondria. Isobutyraldehyde is assumed to be membrane permeable. Isobutyraldehyde produced in the mitochondrial matrix can transverse the mitochondrial membrane and can be reduced to isobutanol by a cytosolic ADH. If an NADH dependent ADH is used in the isobutanol pathway, localization of said ADH to the cytosol instead of the mitochondrial matrix can reduce the flux through the NADH shuttle pathways by half. In this case 50% of the reduced cofactor consumed in the isobutanol pathway is regenerated in the same compartment where it is oxidized. This may improve productivity of an isobutanol production strain. This approach of expressing the isobutyraldehyde reducing activity in the cytosol is also viable for NADPH dependent ADHs, since most of the NADPH turnover in yeast cells occurs in the cytosol.

In yeast, an NADPH dependent KARI is expressed endogenously. As the endogenous enzyme could also function in the mitochondrial isobutanol pathway and consume NADPH, it would results in an imbalance in the cofactor usage. Therefore, the gene encoding this enzyme would be deleted and the activity complemented by the NADH dependent enzyme. Native yeast Ilv5p has two functions. One is the catalytic KARI activity and the other is stabilization of mitochondrial DNA. Many KARIs such as for example the bacterial homologs do not have the DNA stabilizing function. If the NADH dependent KARI that is used in the isobutanol producing yeast does not provide the DNA stabilizing function a mutant ILV5 which has lost its catalytic KARI activity but maintains its DNA stabilizing functionality can be used to ensure mitochondrial stability. Such mutants are known in the art. Alternatively a host strain containing wild-type ILV5 can be used to maintain mitochondrial DNA stability with a NADH dependent KARI overexpressed in addition. Exemplary NADH-dependent KARI enzymes are discussed and described in the commonly owned and co-pending U.S. application Ser. No. 12/610,784, hereby incorporated by reference in its entirety. There are two NADPH dependent alcohol dehydrogenases, Adh6p and Adh7p in S. cerevisiae, that are able to convert isobutyraldehyde to isobutanol. However, these enzymes are likely expressed in the cytosol of yeast and should not affect the cofactor balance in the mitochondria.

In one embodiment of the invention, a microorganism is provided in which the cofactor-dependent final step for the conversion of isobutyraldehyde to isobutanol is catalyzed by an NADH dependent alcohol dehydrogenase. In one specific embodiment, such an alcohol dehydrogenase may be encoded by the Drosophila melanogaster alcohol dehydrogenase (Accession: NT_{—}033779, Region: 14615555.14618902) (SEQ ID NO: 161) or homologs thereof. In another specific embodiment, such an alcohol dehydrogenase may be encoded by Lactococcus lactis adhA codon optimized for S. cerevisiae (Ll_adhA_coSc-1) (SEQ ID NO: 67) coding for L. lactis AdhA (SEQ ID NO: 68).

In one embodiment of this invention only one of the redox cofactor dependent conversions of the isobutanol pathway is catalyzed by an enzyme that is NADH dependent while the other enzyme is NADPH dependent. In a specific aspect the enzyme that is NADH dependent is the ADH converting isobutyraldehyde to isobutanol and the NADPH dependent enzyme is KARI converting acetolactate into dihydroxyisovalerate. This partial cofactor balance improves the yield of isobutanol production of a recombinant microorganism expressing an isobutanol pathway.

### Microorganism Characterized by Providing Cofactor Balance Via Malate Pathway

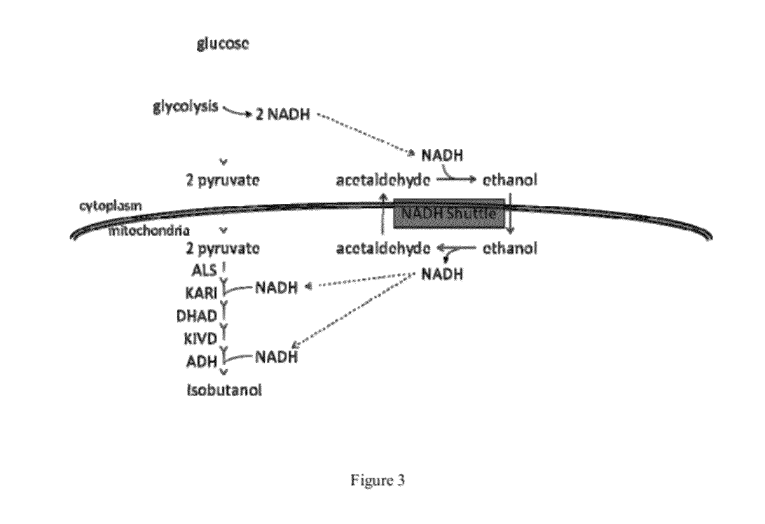

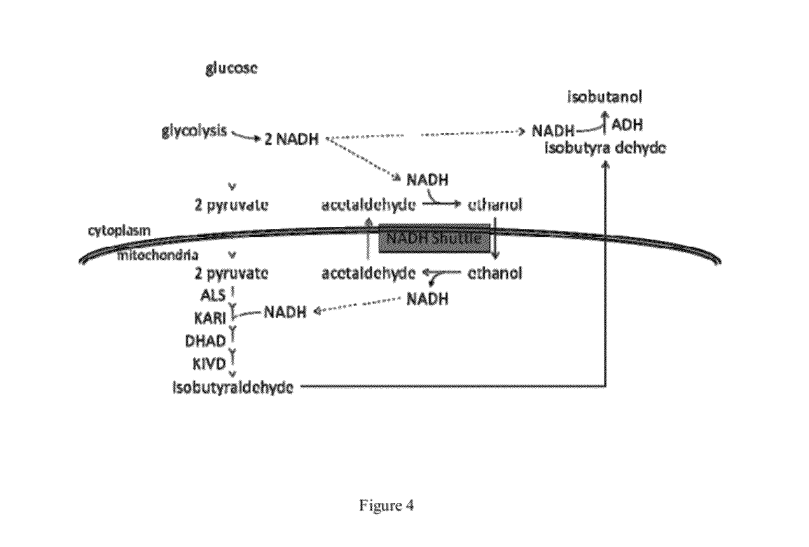

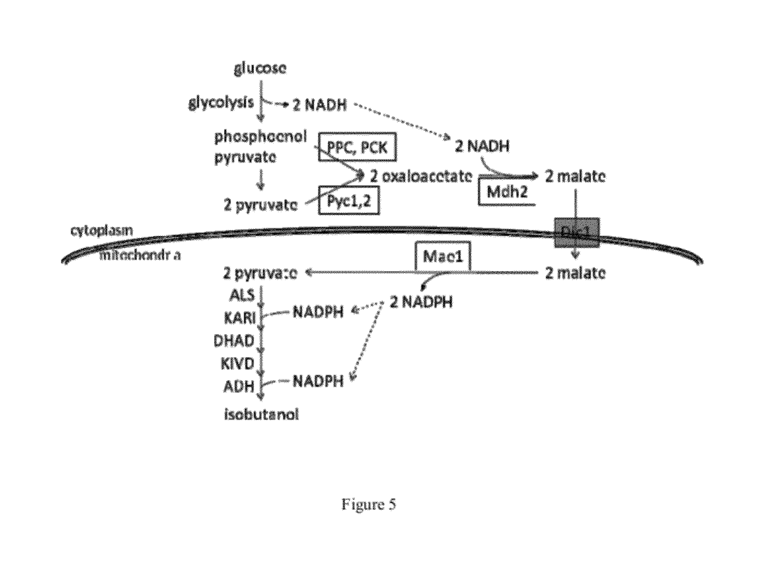

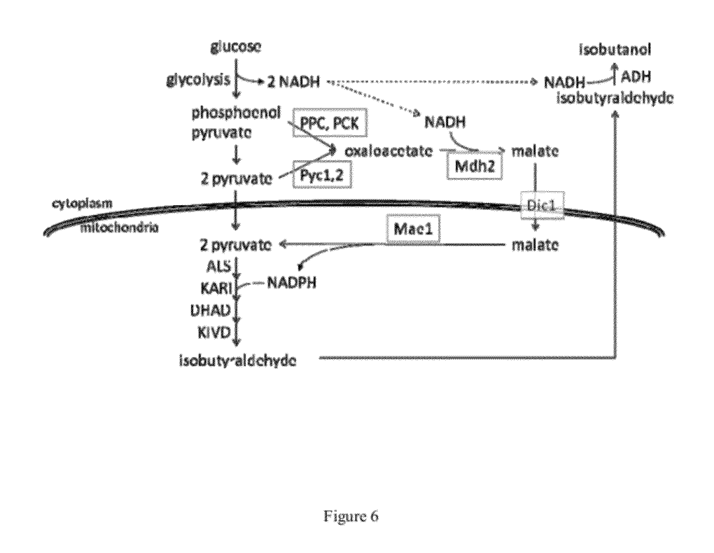

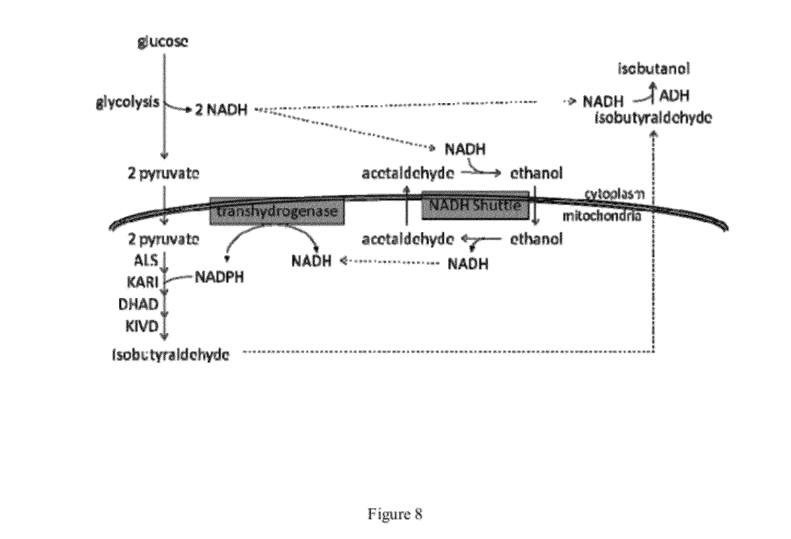

Production of isobutanol from glucose using the aforementioned pathway in the mitochondria results in cofactor imbalance as 2 NADHs are generated in the cytosol via glycolysis and 1 or 2 NADPHs are consumed in the mitochondria by the isobutanol pathway. Whether 1 or 2 redox cofactors are consumed in the mitochondria depends on the isobutanol pathway. If pyruvate is converted to isobutanol in the mitochondria as illustrated in FIGS. 2, 3, and 5 then 2 NAPH are consumed in this compartment. If the conversion of isobutyraldehyde to isobutanol takes place in the cytosol as is the case for the pathways illustrated in FIGS. 4, 6, 8, and 10 then one NADPH is consumed in the mitochondria. One approach to balance these cofactors is to introduce a bypass in which the NADHs in the cytosol is oxidized to generate a compound that is able to pass into the mitochondria and then for that compound to be utilized to reduce an NADP+ in the mitochondria. One such compound is malate. Malate can be transported into the mitochondria via the dicarboxylate carrier, Dic1p. Malate is generated in the cytosol from either pyruvate or phosphoenol pyruvate (PEP) via an intermediate, oxaloacetate (OAA). OAA is produced from pyruvate via Pyruvate carboxylase (Pyc1p or Pyc2p) or alternatively from PEP via phosphoenolpyruvate carboxylase (E. coli Ppc) or phosphoenolpyruvate carboxylkinase (Pck1). The conversion of OAA to malate by a malate dehydrogenase (Mdh2p) reoxidizes a cytosolic NADH. Malate in the mitochondria can then be converted to pyruvate using the mitochondrial malic enzyme, Mae1p, which in the process reduces an NADP+, thus generating a mitochondrial NADPH. Since 2 malate can be produced per glucose, 2 cytosolic NADHs are converted into 2 mitochondrial NADPHs by the use of this bypass.

Microorganism Characterized by Providing Cofactor Balance Via a Transhydrogenase

As detailed above, production of isobutanol from glucose using the aforementioned pathway in the mitochondria results in cofactor imbalance as 2 NADHs are generated in the cytosol via glycolysis and 2 NADPHs are consumed in the mitochondria by the isobutanol pathway. Yeast cells are able to transfer the reducing equivalents from the cytosol to the mitochondria via an NADH shuttle. This results in an NADH in the mitochondria, which would still result in a cofactor imbalance with the isobutanol pathway. This imbalance can be resolved by the use of a transhydrogenase.

Yeast do not contain transhydrogenases. The heterologous expression of bacterial, plant or other eukaryotic transhydrogenases in yeast can be used to provide cofactor balance. Previous attempts to express heterologous transhydrogenases in yeast resulted in the conversion of NADPH to NADH. The soluble transhydrogenase from Azotobacter vinelandii was functionally expressed in S. cerevisiae (Nissen, T L, et al. 2000 Yeast 16, 463-474.) to introduce an alternative pathway for the reoxidation of NADH with the goal of reducing glycerol production. The approach was unsuccessful because of the catalysis of the opposite reaction by the heterologous transhydrogenase. It was reported that the native soluble transhydrogenase in E. coli (SthA) catalyze the conversion of NADPH to NADH (Sauer, U., et al., 2004 The Journal of Biological Chemistry 279, 6613-6619.) The membrane bound transhydrogenase from E. coli coded by pntA and pntB was functionally expressed in S. cerevisiae (Anderlund et al., 1999 Applied and Environmental Microbiology 65, 2333-2340.) and it was observed that this enzyme also catalyzed the conversion of NADPH to NADH and hence was not useful for the reoxidation of NADH in the strains that were constructed. It was found that the transhydrogenase was inserted into the endoplasmic reticulum membrane. This targeting is the likely reason for the failure of the transhydrogenase to catalyze reoxidation of NADH, which is its native function in E. coli (Sauer, U., et al., The Journal of Biological Chemistry 2004 279 6613-6619.). The transhydrogenases that natively convert NADH to NADPH are generally membrane proteins that use the proton motive force to drive the reaction they are catalyzing. Bacterial transhydrogenases are in the cell membrane while plant and mammalian transhydrogenases are located in the inner mitochondrial membrane. For the heterologous transhydrogenase expression these enzymes can be targeted either to the cytoplasmic membrane or to the mitochondrial membrane in yeast. To achieve this leader sequences have to be added to the heterologous proteins. The mechanism of membrane targeting is well understood and the direction of normally cytosolic proteins to the mitochondria has been demonstrated. These targeting mechanisms are well conserved throughout the eukaryotes as demonstrated by the use of plant mitochondrial targeting sequences in yeast. Eukaryotic transhydrogenases can be expressed in yeast with their native targeting and sorting sequences. Bacterial transhydrogenases can be fused to mitochondrial targeting and membrane sorting sequences that have been characterized in yeast inner membrane proteins. For the expression of a transhydrogenase in yeast a fungal source organism is preferred and among the fungi an ascomycete is preferred. Several transhydrogenases have been found by homology searches. Preferred source organisms for the transhydrogenase include Neurospora crassa, Aspergillus clavatus, Aspergillus oryzae, Aspergillus Niger, Aspergillus fumigates, Aspergillus terreus, Phaeosphaeria nodorum, Coccidioides immitis, Neosartorya fischeri, Magnaporthe grisea, Ajellomyces capsulate, Botryotinia fuckeliana, Sclerotinia sclerotiorum, Podospora anserine, and Pyrenophora tritici-repentis. In a preferred embodiment the transhydrogenase of Neurospora crassa (GI:164426165) is expressed in yeast to achieve cofactor balance of a mitochondrially produced isobutanol pathway. The gene codes for a NAD(P) transhydrogenase, mitochondrial precursor and consists of two subunits alpha and beta coded by the regions PNTA and PNTB.

A preferred transhydrogenase under conditions in which the reduced cofactor NADPH is limiting is one that preferentially catalyzes the conversion of NADH to NADPH. For example, membrane-bound transhydrogenases have been described in eukaryotes as for example in mammalian cells (Rydstrom J., Trends in Biochemical Sciences, 2006, 31(7): 355-358) as well as in bacteria such as E. coli (Sauer U., et al., The Journal of Biological Chemistry 2004, 279: 6613-6619). Membrane bound transhydrogenases require energy in form of proton translocation to catalyze the reaction. As long as there is enough energy available to maintain the proton gradient across the cell membrane or across the inner mitochondrial membrane a transhydrogenase may thus be used to balance an otherwise imbalanced metabolic pathway. Thus, expression of these transhydrogenases in the mitochondria of yeast can resolve the cofactor imbalance in the mitochondria.

Microorganism Characterized by Providing Partial Cofactor Balance Via ADH that is Able to Utilize NADH.

As detailed above, production of isobutanol from glucose using the aforementioned pathway in the mitochondria results in cofactor imbalance as 2 NADHs are generated in the cytosol via glycolysis and 2 NADPHs are consumed in the mitochondria by the isobutanol pathway. Yeast cells are able to transfer the reducing equivalents from the cytosol to the mitochondria via an NADH shuttle. This results in an NADH in the mitochondria, which would still result in a cofactor imbalance with the isobutanol pathway. This imbalance can be partially resolved by the use of a mitochondrial isobutanol pathway that consumes 1 NADH and 1 NADPH instead of 2 NADPH. For the mitochondrial isobutanol pathway to consume NADH, the NADPH dependent enzyme, ADH can be replaced by either an NADH dependent homolog or an enzyme that has been engineered to use NADH.

There are two NADPH dependent alcohol dehydrogenases, Adh6p and Adh7p in S. cerevisiae, that are able to convert isobutyraldehyde to isobutanol. However, these enzymes are likely expressed in the cytosol of yeast and should not affect the cofactor balance in the mitochondria much as long as the conversion of isobutyraldehyde to isobutanol takes place in that compartment. If the reduction of isobutyraldehyde is catalyzed by a cytosolic NADH dependent ADH the endogenous NADPH dependent enzymes might interfere with the cofactor balance depending on the expression levels of these endogenous enzymes. In case of interference the endogenous ADH activities can be reduced or eliminated for example by disruption of the native coding genes.

In one embodiment of the invention, a microorganism is provided in which the cofactor-dependent final step for the conversion of isobutyraldehyde to isobutanol is catalyzed by an NADH dependent alcohol dehydrogenase. In a specific aspect, such an alcohol dehydrogenase may be encoded by the Drosophila melanogaster alcohol dehydrogenase (Accession: NT_{—}033779, Region: 14615555..14618902) or homologs thereof. In another specific aspect such an alcohol dehydrogenase may be Lactococcus lactis AdhA (SEQ ID NO: 68).

In one embodiment of this invention only one of the redox cofactor dependent conversions of the isobutanol pathway is catalyzed by an enzyme that is NADH dependent while the other enzyme is NADPH dependent. In a specific aspect the enzyme that is NADH dependent is the ADH converting isobutyraldehyde to isobutanol and the NADPH dependent enzyme is KARI converting acetolactate into dihydroxyisovalerate. This partial cofactor balance improves the yield of isobutanol production of a recombinant microorganism expressing an isobutanol pathway.

A partial cofactor balance as that achieved by use of a NADH dependent ADH in the isobutanol pathway can be combined with the use of the malate pathway and it can be combined with the expression of a transhydrogenase. These combined approaches can lead to complete cofactor balance of the production microorganism.

### Microorganism Characterized by Increased Capacity to Produce Intermediates of the Isobutanol Pathway

As a consequence of increased yield of isobutanol, it follows that this yeast microorganism exhibits a higher capacity to produce the intermediates of the isobutanol pathway including, but not limited to, acetolactate, 2,3-dihydroxyisovalerate, keto-isovalerate, and isobutyraldehyde.

### Method of Using Microorganism for High-Yield Isobutanol Fermentation

In a method to produce isobutanol from a carbon source at high yield, the yeast microorganism is cultured in an appropriate culture medium containing a carbon source.

Another exemplary embodiment provides a method for producing isobutanol comprising a recombinant yeast microorganism of the invention in a suitable culture medium containing a carbon source that can be converted to isobutanol by the yeast microorganism of the invention.

In certain embodiments, the method further includes isolating isobutanol from the culture medium. For example, isobutanol may be isolated from the culture medium by any method known to those skilled in the art, such as distillation, pervaporation, or liquid-liquid extraction. The GIFT® separation process is discussed and described in the commonly owned and co-pending U.S. patent application Ser. No. 12/342,992 (US Publication No. 20090171129), hereby incorporated by reference in its entirety.

### EXAMPLES

The following examples illustrate how yeast microorganisms are modified to produce isobutanol pathway enzymes in their mitochondria, allowing for the production of isobutanol under conditions that include anaerobic conditions.

### Sample Preparation

All samples (2 mL) from fermentation experiments performed in shake flasks are centrifuged at 14,000×g for 10 min and the supernatant is stored at 4° C. for later analysis. Analysis of substrates and products is performed using authentic standards (>99%, obtained from Sigma-Aldrich), and a 5-point calibration curve (with 1-pentanol as an internal standard for analysis by gas chromatography).

Determination of Optical Density and Cell Dry Weight The optical density of the yeast cultures is determined at 600 nm using a DU 800 spectrophotometer (Beckman-Coulter, Fullerton, Calif., USA). Samples are diluted as necessary to yield an optical density of between 0.1 and 0.8. The cell dry weight is determined by centrifuging 50 mL of culture prior to decanting the supernatant. The cell pellet is washed once with 50 mL of milliQ H₂O, centrifuged and the pellet is washed again with 25 mL of milliQ H₂O. The cell pellet is then dried at 80° C. for at least 72 hours. The cell dry weight is calculated by subtracting the weight of the centrifuge tube from the weight of the centrifuge tube containing the dried cell pellet.

### Gas Chromatography

Analysis of volatile organic compounds, including ethanol and isobutanol is performed on a HP 5890 gas chromatograph fitted with an HP 7673 Autosampler, a DB-FFAP column (J&W; 30 m length, 0.32 mm ID, 0.25 μM film thickness) or equivalent connected to a flame ionization detector (FID). The temperature program is as follows: 200° C. for the injector, 300° C. for the detector, 100° C. oven for 1 minute, 70° C./minute gradient to 235° C., and then hold for 2.5 min.

Analysis is performed using authentic standards (>99%, obtained from Sigma-Aldrich), and a 5-point calibration curve with 1-pentanol as the internal standard.

### High Performance Liquid Chromatography

Analysis of glucose and organic acids is performed on a HP-1100 High Performance Liquid Chromatography system equipped with an Aminex HPX-87H Ion Exclusion column (Bio-Rad, 300×7.8 mm) or equivalent and an H⁺ cation guard column (Bio-Rad) or equivalent. Organic acids are detected using an HP-1100 UV detector (210 nm, 8 nm 360 nm reference) while glucose is detected using an HP-1100 refractive index detector. The column temperature is 60° C. This method is Isocratic with 0.008 N sulfuric acid in water as mobile phase. Flow is set at 0.6 mL/min. Injection size is 20 μL and the run time is 30 minutes.

### Molecular Biology and Bacterial Cell Culture

Standard molecular biology methods for cloning and plasmid construction are generally used, unless otherwise noted (Sambrook, J., Russel, D. W. Molecular Cloning, A Laboratory Manual. 3 ed. 2001, Cold Spring Harbor, N.Y.: Cold Spring Harbor Laboratory Press).

Standard recombinant DNA and molecular biology techniques used in the Examples are well known in the art and are described by Sambrook, J., Russel, D. W. Molecular Cloning, A Laboratory Manual. 3 ed. 2001, Cold Spring Harbor, N.Y.: Cold Spring Harbor Laboratory Press; and by T. J. Silhavy, M. L. Bennan, and L. W. Enquist, Experiments with Gene Fusions, Cold Spring Harbor Laboratory Press, Cold Spring Harbor, N.Y. (1984) and by Ausubel, F. M. et al., Current Protocols in Molecular Biology, pub. by Greene Publishing Assoc. and Wiley-Interscience (1987).

General materials and methods suitable for the routine maintenance and growth of bacterial cultures are well known in the art. Techniques suitable for use in the following examples may be found as set out in Manual of Methods for General Bacteriology (Phillipp Gerhardt, R. G. E. Murray, Ralph N. Costilow, Eugene W. Nester, Willis A. Wood, Noel R. Krieg and G. Briggs Phillips, eds.), American Society for Microbiology, Washington, D.C. (1994)) or by Thomas D. Brock in Biotechnology: A Textbook of Industrial Microbiology, Second Edition, Sinauer Associates, Inc., Sunderland, Mass. (1989).

### Aerobic Batch Fermentations

3 mL overnight cultures in YPD are inoculated from colonies or patches of the strains to be tested. These overnight cultures are incubated in 14 mL culture tubes for 18 h at 30° C., in an orbital shaker at 250 rpm. These overnight cultures are then used to inoculate 100 mL YPD cultures in 1 L shake flasks. In the case of a PDC-deficient (Pdc-minus) C2-dependent S. cerevisiae strain, the overnight culture and the 100 mL culture are grown in YPEthanol (20 g-ethanol L⁻¹). The cultures are harvested at an OD600 of 0.6-1 in mid- to late-log phase. The cells are resuspended in 50 mL YPD medium containing 50 g/L glucose and the cultures are incubated in 250 mL shake flasks at 30° C., 250 rpm. Samples (2 mL) are taken at 24 and 48 hours and cells removed by centrifugation at ≧14000×g for 10 min in a microcentrifuge. The supernatants are kept at 4° C. until analysis by Gas Chromatography and/or High Performance Liquid Chromatography.

### Anaerobic Batch Fermentations

Anaerobic batch cultivations are performed at 30° C. in stoppered 100 mL serum bottles that are inoculated and sampled in an anaerobic chamber to maintain anaerobic conditions throughout the experiment. A 20 mL aliquot of medium with an initial glucose concentration of 20 g-glucose L⁻¹ is used (Kaiser et al., Methods in Yeast Genetics, a Cold Spring Harbor Laboratory Manual (1994)). Samples (2 mL) are taken at 24 and 48 hours. The fermentation is ended after 48 hours or when all glucose is consumed. Samples are processed and analyzed by Gas Chromatography and/or High Performance Liquid Chromatography as described above.

Yeast Transformations—S. cerevisiae

S. cerevisiae strains were transformed by the Lithium Acetate method (Gietz et al., Nucleic Acids Res. 27:69-74 (1992)). Cells from 50 mL YPD cultures were collected by centrifugation (2700 rcf, 2 minutes, 25° C.) once the cultures reached an OD₆₀₀ of 1.0. The cells were washed cells with 50 mL sterile water and collecte^{d} by centrifugation at 2700 rcf for 2 minutes at 25° C. The cells were washed again with 25 mL sterile water and cells were collected by centrifugation at 2700 rcf for 2 minutes at 25° C. The cells were resuspended in 1 mL of 100 mM lithium acetate and transferred to a 1.5 mL Eppendorf tube. The cells were collected by centrifugation for 20 sec at 18,000 rcf, 25° C. The cells were resuspended in a volume of 100 mM lithium acetate that was approximately 4× the volume of the cell pellet. A mixture of DNA (final volume of 15 μl with sterile water), 72 μl 50% PEG, 10 μl 1 M lithium acetate, and 3 μl denatured salmon sperm DNA was prepared for each transformation. In a 1.5 mL tube, 15 μl of the cell suspension was added to the DNA mixture (85 n1), and the transformation suspension was vortexed with 5 short pulses. The transformation was incubated at 30 minutes at 30° C., followed by incubation for 22 minutes at 42° C. The cells were collected by centrifugation for 20 sec at 18,000 rcf, 25° C. The cells were resuspended in 100 μl SOS (1 M sorbitol, 34% (v/v) YEP (1% yeast extract, 2% peptone), 6.5 mM CaCl₂) and spread over a SC+glucose-uracil plate.

### Yeast Colony PCR

Yeast colony PCR is performed using the Epicentre Failsafe PCR kit (using Buffer E). Reactions are set up according to manufacturer's protocol and a small amount of yeast cells are resuspended in the reaction. PCR reactions are run according to standard protocols.

Preparation of E. coli Electrocompetent Cells and Transformation

The acceptor strain culture is grown in SOB-medium (Sambrook, J., Russel, D. W. Molecular Cloning, A Laboratory Manual. 3 ed. 2001, Cold Spring Harbor, N.Y.: Cold Spring Harbor Laboratory Press) to an OD₆₀₀ of about 0.6 to 0.8. The culture is concentrated 100-fold, washed once with ice cold water and 3 times with ice cold 10% glycerol. The cells are then resuspended in 150 μL of ice-cold 10% glycerol and aliquoted into 50 μL portions. These aliquots are used immediately for standard transformation or stored at −80° C. These cells are transformed with the desired plasmid(s) via electroporation. After electroporation, SOC medium (Sambrook, J., Russel, D. W. Molecular Cloning, A Laboratory Manual. 3 ed. 2001, Cold Spring Harbor, N.Y.: Cold Spring Harbor Laboratory Press) is immediately added to the cells. After incubation for an hour at 37° C. the cells are plated onto LB-plates containing the appropriate antibiotics and incubated overnight at 37° C.

Transformation of Kluyveromyces marxianus

For the integration of the isobutanol pathway into the chromosome of the yeast strains a modular cassette approach is taken. Two plasmids are constructed wherein plasmid 1 contains from 5′ to 3′: P_{PDC1} promoter, pathway genes with their promoters and terminators, 5′ end of URA3 with its promoter. Plasmid 2 contains from 5′ to 3′:3′ end of URA3 with its terminator, pathway genes with their promoters and terminators, PDC1 terminator. The URA3 sequences overlap to provide homology between the two plasmids. These features of the plasmids can be cut out of the vectors using restriction enzymes or amplified by PCR to get linear DNA pieces. These pieces are then transformed into the host organism. Homologous recombination leads to integration of both DNA pieces into the PDC1 locus on the chromosome deleting PDC1. At the same time the URA3 pieces in each linear DNA fragment recombine to give a functional URA3 marker that is used for selection.

K. marxianus is grown in YPD medium at 30° C. and 250 rpm to an OD600 of 1.0-4.0. The cells are pelleted and washed with 10 mL of EB (10 mM Tris-Cl, 270 mM sucrose, 1 mM MgCl2, pH 7.5). The cells are again pelleted, and then resuspended in 10 mL IB (YPD+ fresh 25 mM DTT, 20 mM HEPES, pH 8.0) and incubated at 30° C. and 250 rpm for 30 minutes. The cells are then pelleted and washed with 10 mL EB, and the cells are kept on ice from this point on. The cells are pelleted and resuspended in 1 mL EB and transferred to a microfuge tube. The cells are pelleted in a microfuge, and then resuspended in the appropriate amount of EB to make the final cell concentration 30-380D/mL. 400 μL of the cell suspension is added to a chilled cuvette (4 mm gap), and 50 μL of linearized DNA (1-2 μg) is added and mixed by pipetting. The cells are incubated in the cuvette with DNA on ice for 15 to 30 minutes, and then electroporated at 1.8 kV, 1000Ω, and 25 μF. The cells are washed out of the cuvette with 1 mL YPD, and transferred to a fresh 50 mL tube and incubated at 30° C. and 250 rpm for 4 hours. After recovery, the transformation is plated on 7 selection plates (200 μL per plate) and incubated at 30° C.

### Construction of Strains and Plasmids

The Kluyveromyces marxianus strain GEVO1068 (NRRL-Y-7571) was obtained from the USDA collection.

GEVO1947 is a ura3Δ version of GEVO1068 and was generated by selection on 5-fluorouracil-6-carboxylic acid monohydrate (FOA). The K. marxianus URA3 gene was deleted by transformation of GEVO1068 with a PCR fragment of Km_URA3 carrying a deletion of 348 bp that was amplified from pGV1799 using primers 394 and 395. The Km_ura3Δ transformants were selected by plating on 5-FOA plates. The 5-FOA resistant colonies were screened for correct phenotype (auxotrophic for uracil) and for the correct genotype by colony PCR using primers 562 and 837. The wild-type Km_URA3 gene would result in a PCR product of 871 bp using this primer pairs while the presence of the deletion allele would result in a product of 527 bp. This analysis identified FOA^{R} transformants with the expected 350 bp PCR product for a strain containing the deletion allele). In addition, several transformants generated a slightly larger band using the same primer pair. This PCR result is consistent with the hypothesis that these transformants contain a smaller deletion (˜200 bp) in the KmURA3. A transformant with the putative smaller deletion was named GEVO1947 (Km_ura3Δ2).

The K. marxianus strain GEVO1969 was generated by deletion of PDC1 in GEVO1947. The K. marxianus PDC1 gene was replaced with a G418R marker (aminoglycoside-3′-phosphotransferase (aph) from Tn5 under the TEF1 promoter) by transformation of GEVO1947 with a disruption cassette which contained from 5′ to 3′, the Km_PDC1 promoter, the G418R marker, and the Km_PDC1 terminator sequences. The Km_PDC1 promoter was amplified from K. marxianus genomic DNA using primers 1671 and 1672. The G418R marker was amplified from pGV1503 using primers 1673 and 1674. The Km_PDC1 terminator was amplified from K. marxianus genomic DNA using primers 1675 and 1676. Primers 1673 and 1674 have 5′ extensions that are complementary to primers 1672 and 1675, respectively. These three fragments were combined via two rounds of SOE PCR to generate the disruption cassette. In the first round of SOE PCR, the Km_PDC1 promoter and the G418R marker were combined using primers 1671 and 1674, and the G418R marker and the Km_PDC1 terminator were combined using primers 1673 and 1676. In the second round of SOE PCR, the Km_PDC1 promoter-G418 marker fragment was combined with the Km_PDC1 terminator using primers 1671 and 1676, and the Km_PDC1 promoter was combined with the G418R marker-Km_PDC1 terminator fragment using primers 1671 and 1676. These two PCR products were pooled together, purified and transformed into GEVO1947. Transformants were selected on YPD+G418 plates. Transformants were confirmed for successful deletion of Km_PDC1 by their inability to grow anaerobically, colony PCR, and a lack of ethanol production. One such clone was named GEVO1969.

The PDC-deficient (Pdc-) S. cerevisiae strain GEVO1581 was obtained from Prof. Paul van Heusden at the University of Leiden in the Netherlands. Strain GEVO1584 was generated by crossing GEVO1187 and GEVO1537 and selecting for diploids from mating between the two strains by selecting for growth on minimal glucose media lacking uracil. The resulting diploids were sporulated and progeny with the appropriate genotype was isolated. Strain GEVO7777 is generated by crossing GEVO1187 and GEVO1537 and selecting for diploids from mating between the two strains by selecting for growth on minimal glucose media lacking uracil. The resulting diploids are sporulated and progeny with the appropriate genotype is isolated.

The C2-independent strain, GEVO1863, was generated from the PDC-deficient (Pdc-) S. cerevisiae GEVO1584 by evolution. This evolution was performed essentially as previously described (van Maris, A., et al., Applied and Environmental Microbiology, 2004, 70(1):159-166). Briefly, GEVO1584 was grown in a chemostat using minimal media with ethanol as a carbon source. The media was switched to minimal media with 7.125 g/L glucose and 0.375 g/L acetate. Once the culture was stabilized, the acetate level was decreased to 0 g/L over a period of 3 weeks. Over the same time period the glucose concentration in the feed was increased so that the carbon concentration in the feed was constant throughout the experiment. A single colony was isolated from the chemostat and was further evolved through 24 serial dilutions in YPD in test tubes. GEVO1863 is an isolate from this evolved culture. This strain does not require ethanol or acetate for growth.

GEVO1803 was made by transforming GEVO1186 with the 6.7 kb pGV1730 (contains S. cerevisiae TRP1 marker and the CUP1 promoter-driven B. subtilis BsalsS2 (SEQ ID NO: 151) that had been linearized by digestion with NruI. Completion of the digest was confirmed by running a small sample on a gel. The digested DNA was then purified using Zymo Research DNA Clean and Concentrator and used in the transformation. Trp+ clones were confirmed for the correct integration into the PDC1 locus by colony PCR using primer pairs 1440+1441 and 1442+1443 for the 5′ and 3′ junctions, respectively. Transcription of BsalsS2 was confirmed by qRT-PCR using primer pairs 1323+1324 (qRT-PCR).

GEVO2107 was made by transforming GEVO1803 with linearized, HpaI-digested pGV1914. Correct integration of pGV1914 at the PDC6 locus was confirmed by analyzing candidate Ura+ colonies by colony PCR using primers 1440 plus 1441, or 1443 plus 1633, to detect the 5′ and 3′ junctions of the integrated construct, respectively. Expression of all transgenes was confirmed by qRT-PCR using primer pairs 1321 plus 1322, 1587 plus 1588, and 1633 plus 1634 to examine BsalsS2, LlkivD2, and DmADH transcript levels, respectively.

GEVO2158 was made by transforming GEVO2107 with NruI-digested pGV1936. Correct integration of pGV1936 at the PDC5 locus was confirmed by analyzing candidate Ura+, Leu+ colonies by colony PCR using primers primers 1436 plus 1437, or 1595 plus 1439, to detect the 5′ and 3′ junctions of the integrated construct, respectively. Expression of all transgenes were confirmed by qRT-PCR using primer pairs 1321 plus 1322, 1597 plus 1598, 1566 plus 1567, 1587 plus 1588, 1633 plus 1634, and 1341 plus 1342 to examine mRNA levels of BsalsS2 (SEQ ID NO: 151), EcilvCco(Sc)^{Q110V} (SEQ ID NO: 159), Scilv3ΔN (SEQ ID NO: 160), LlkivD2 (SEQ ID NO:155), (SEQ ID NO: 161), and ACT1, respectively.

GEVO2302 was made by sporulating GEVO2158. Haploid spores were prepared for random spores analysis (as described above), and the spores were plated onto SCE-Trp, Leu, Ura medium. Candidate colonies were patched onto SCE-Trp, Leu, Ura plates and then replica plated onto YPD and YPE plates. Patches that grew on YPE but failed to grow on YPD were further analyzed by colony PCR to confirm mating type (and, hence, their status as haploid). Several verified haploid candidates were further analyzed for transgenic expression by qRT-PCR. GEVO2302 contains the full isobutanol pathway, and specifically contains a transgenic expressing EcilvCco(Sc)^{Q110V}.

GEVO2542 was made by transforming GEVO1969 with pGV2061 digested with EcoRI and BglII and pGV2062 digested with NheI and HindIII. The integration was confirmed by qRT-PCR which confirmed transcription of Bs_alsS, Ll_kivd and Dm_ADH. From this strain the ura3 marker was removed by selection on 5-FOA containing plates. Colonies from the plate were streaked on YPE and SCD-ura plates to confirm the loss of the ura3 marker. One confirmed clone was named GEVO2542.

The K. marxianus strains GEVO2346, GEVO2347, and GEVO2348 were constructed by transformation of GEVO1969 with pGV1990 and pGV2015 that had been linearized with the restriction enzyme PvuI and purified by ethanol precipitation. The transformation was plated on SCE-URA plates to select for integrants. Integrants were verified by colony PCR.

The K. marxianus strains GEVO2276 and GEVO2277 were constructed by transformation of KARL1969 with pGV1875 that had been linearized with the restriction enzyme PvuI and purified by ethanol precipitation. The transformation was plated on SCE-URA plates to select for integrants. Integrants were verified by colony PCR.

The K. marxianus strains GEVO2087 and GEVO2088 were constructed by transformation of KARL1947 with pGV1875 that had been linearized with the restriction enzyme PvuI. The transformation was plated on SC-URA plates to select for integrants. Integrants were verified by colony PCR.

The Saccharomyces cerevisiae strain GEVO2072 was constructed by transformation of GEVO1186 with pGV1875 that had been linearized with the restriction enzyme PvuI. The transformation was plated on SC-URA plates to select for integrants. Integrants were verified by colony PCR.

The Saccharomyces cerevisiae strain GEVO2119 was constructed by transformation of GEVO1186 with pGV1874 that had been linearized with the restriction enzyme PvuI. The transformation was plated on SC-URA plates to select for integrants. Integrants were verified by colony PCR.

The Saccharomyces cerevisiae strains GEVO2120 and GEVO2121 were constructed by transformation of GEVO1186 with pGV1877 that had been linearized with the restriction enzyme PvuI. The transformation was plated on SC-URA plates to select for integrants. Integrants were verified by colony PCR.

The Saccharomyces cerevisiae strains GEVO2122 and GEVO2123 were constructed by transformation of GEVO1186 with pGV1878 that had been linearized with the restriction enzyme PvuI. The transformation was plated on SC-URA plates to select for integrants. Integrants were verified by colony PCR.

The Saccharomyces cerevisiae strains GEVO2124 and GEVO2125 were constructed by transformation of GEVO1186 with pGV1879 that had been linearized with the restriction enzyme PvuI. The transformation was plated on SC-URA plates to select for integrants. Integrants were verified by colony PCR.

The Saccharomyces cerevisiae strain GEVO2126 was constructed by transformation of GEVO1186 with pGV1892 that had been linearized with the restriction enzyme PvuI. The transformation was plated on SC-URA plates to select for integrants. Integrants were verified by colony PCR.

Strain AP is constructed by transforming GEVO1947 with linear DNA from pGV1817 and additional linear DNA from pGV7001 resulting in strain AP. This combination of linear DNA contains genes coding for all five isobutanol pathway enzymes, and the uracil marker for selection. The transformation leads to random (non-targeted) insertion of the isobutanol pathway into the K. marxianus genome. Adh7p is targeted to its native compartment, the cytosol. All other pathway enzymes are targeted to the mitochondrium. Pathway enzymes that are not natively localized to the mitochondrium are fused to mitochondrial targeting sequences that direct them to the mitochondrium. The transformed cells are plated onto selective medium without uracil and incubated at 30° C. for 3 days. After 3 days colonies are patched onto selective plates and these patches are used for colony PCR and RT PCR to verify correct integration and transcription of the pathway genes.

To construct strain SCP1, GEVO1186, a diploid CEN.PK strain, is first transformed with linear DNA from pGV1817 and additional linear DNA from pGV7001 resulting in strain SCP1. The combination of linear DNA contains genes coding for all five isobutanol pathway enzymes, the S. cerevisiae PDC1 promoter (P_{ScPDC1}) and terminator (T_{ScPDC1}) sequences for homologous integration into the PDC1 locus and the uracil marker for selection. This homologous replacement event results in the simultaneous integration of the isobutanol pathway along with the deletion of the PDC1 coding sequence. Adh7p is targeted to its native compartment, the cytosol. All other pathway enzymes are targeted to the mitochondrium. Pathway enzymes that are not natively localized to the mitochondrium are fused to mitochondrial targeting sequences that direct them to the mitochondrium. The transformed cells are plated onto selective medium using ethanol as carbon source and lacking uracil and incubated at 30° C. for 3-4 days. After 3-4 days colonies are patched onto selective plates and these patches are used for colony PCR and RT PCR to verify correct integration and transcription of the pathway genes.

To construct strain SC1, GEVO7777, a haploid CEN.PK strain deleted for PDC5 and PDC6, is first transformed with linear DNA from pGV1817 and additional linear DNA from pGV7001 resulting in strain SC1. The combination of linear DNA contains genes coding for all five isobutanol pathway enzymes, the S. cerevisiae PDC1 promoter and terminator sequences for homologous integration into the PDC1 locus and the uracil marker for selection. This homologous replacement event results in the simultaneous integration of the isobutanol pathway along with the deletion of the PDC1 coding sequence. Adh7p is targeted to its native compartment, the cytosol. All other pathway enzymes are targeted to the mitochondrium. Pathway enzymes that are not natively localized to the mitochondrium are fused to mitochondrial targeting sequences that direct them to the mitochondrium. The transformed cells are plated onto selective medium using ethanol as carbon source and lacking uracil and incubated at 30° C. for 3-4 days. After 3-4 days colonies are patched onto selective plates and these patches are used for colony PCR and RT PCR to verify correct integration and transcription of the pathway genes.

The plasmid pGV1773 (SEQ ID NO: 1) was a clone obtained from DNA2.0 in which the sequence containing the ScPDC1 promoter (P_{ScPDC1}), BsalsS, ScTDH3 promoter (P_{ScTDH3}), LlkivD, ScADH1 promoter (P_{ScADH1}), ScADH7, ScFBA1 promoter (P_{ScFBA1}), and 5′ fragment of ScURA3 was synthesized. BsalsS, LlkivD and ScADH7 have been codon optimized for S. cerevisiae.

The plasmid pGV1774 (SEQ ID NO: 2) was a clone obtained from DNA2.0 in which the sequence containing the 3′ fragment of ScURA3 coding sequence and terminator, ScFBA1 promoter (P_{ScFBA1}), EcilvC codon optimized for S. cerevisiae (EcilvCco), ScTPI1 promoter (P_{ScTPI1}), EcilvD codon optimized for S. cerevisiae (EcilvDco), ScPDC1 terminator (T_{ScPDC1}) was synthesized.

The plasmid pGV1810 (SEQ ID NO: 3) was constructed to replace the EcilvCco gene in pGV1774 with Saccharomyces cerevisiae ILV5. It was constructed by PCR amplification of ILV5 from S. cerevisiae genomic DNA with primers 1615 and 1616, which add XhoI and BamHI sites flanking the full-length ILV5 sequence. The ScILV5 PCR product was digested with XhoI and BamHI. The vector pGV1774 was digested with XhoI and BglII. The digested ScILV5 PCR product and pGV1774 were ligated to yield pGV1810.

The plasmid pGV1811 (SEQ ID NO: 4) was constructed to replace the existing S. cerevisiae P_{ScPDC1} promoter region in pGV1773 with a Kluyveromyces marxianus P_{KmPDC1} promoter region. It was constructed by PCR amplification of a region of the K marxianus P_{PDC1} promoter from genomic DNA with primers 1658 and 1608, and of a region of pGV1773 with primers 1652 and 1653. The above PCR products were combined in a splicing by overlap extension (SOE) PCR reaction to splice the fragments as described by Horton, R M; Hunt, H D; Ho, S N; Pullen, J K; Pease, L R. Engineering hybrid genes without the use of restriction enzymes: gene splicing by overlap extension. Gene 77, 61-68 (1989). The SOE PCR reaction was required to introduce a BglII site to the 5′ end of the P_{PDC1} promoter. The resulting SOE PCR product and the vector, pGV1773, were digested with PvuI and SalI and ligated to yield pGV1811.

The plasmid pGV1812 (SEQ ID NO: 5) was constructed to replace the existing S. cerevisiae T_{ScPDC1} terminator region in pGV1774 with a K. marxianus T_{KmPDC1} terminator region. It was constructed by PCR amplification of a region of T_{KmPDC1} from genomic DNA with primers 1609 and 1610. The resulting PCR product and the vector, pGV1774, were digested with BamHI and HindIII and ligated to yield pGV1812.

The plasmid pGV1817 (SEQ ID NO: 6) was constructed to replace the EcilvDco gene in pGV1810 with S. cerevisiae ILV3. It was constructed by PCR amplification of ScILV3 from S. cerevisiae genomic DNA using primers 1617 and 1618. The PCR product and pGV1810 were digested with SalI and BamHI, and were ligated to yield pGV1817.

The plasmid pGV1832 (SEQ ID NO: 7) was constructed to replace the existing S. cerevisiae T_{ScPDC1} terminator region in pGV1817 with a K. marxianus T_{KmPDC1} terminator region. It was constructed by digestion of pGV1812 and pGV1817 with PvuI and BamHI. The T_{KmPDC1} from pGV1812 was ligated into the pGV1817 backbone to yield pGV1832.

The plasmid pGV1834 (SEQ ID NO: 8) was constructed to replace the existing Bacillus subtilis BsalsS gene and the Lactococcus lactis LlkivD gene in pGV1811 with the same genes, but with additional sequence encoding the 25 amino acid mitochondrial targeting sequence from the S. cerevisiae cytochrome oxidase subunit 4 gene (COX4) at the 5′ end of BsalsS and additional sequence encoding the mitochondrial targeting sequence from S. cerevisiae Hmi1p, a mitochondrial DNA helicase at the 3′ end of LlkivD. It was constructed by PCR amplification of BsalsS, S. cerevisiae P_{ScTDH3} promoter, and L. lactis LlkivD from pGV1773 with primers 1681 and 1661. Primer 1681 added nucleotides that encode part of the mitochondrial targeting sequence of COX4 from S. cerevisiae to the 5′ end of BsalsS, and primer 1661 added 55 nucleotides encoding part of the mitochondrial targeting sequence of ScHMI1p, to the 3′ end of the LlkivD gene. The above PCR product was then used in an SOE PCR reaction to splice each mitochondrial targeting sequence to the appropriate end of the PCR product. The DNA of mitochondrial targeting sequences was obtained from oligonucleotides of both strands of each mitochondrial targeting sequence. For each mitochondrial targeting sequence, both oligonucleotides were annealed to each other by reconstituting each oligonucleotide to a final concentration of 20 μM in TE (Tris 10 mM, EDTA 1 mM, 50 mM NaCl). 50 μL of each oligonucleotide was mixed with 50 μL of the corresponding oligonucleotide and incubated at 95° C. for 5 minutes in a heating block, and then cooled slowly to room temperature by turning the heating block off. The oligonucleotide numbers for the Cox4p mitochondrial targeting sequence are 1665 and 1666, and for the Hmi1p MTS are 1663 and 1664. The annealed oligonucleotides of the mitochondrial targeting sequences were combined with the PCR product in the SOE PCR reaction in which primers 1683 and 1684 were used. The resulting SOE PCR product was then digested with NheI and XhoI, and the vector, pGV1811, was digested with SalI and AvrII, and the two were ligated to yield pGV1834. The same SOE PCR product is also ligated into pGV1773 using the same restriction endonucleases to yield pGV7001 (SEQ ID NO: 9).

The plasmids pGV7101 (SEQ ID NO: 10) and pGV9834 (SEQ ID NO: 11), are constructed by ligating a SOE-PCR product containing nucleotides coding for the MTS of Adh3p from S. cerevisiae fused to the 5′ end of the ScADH7 gene into the plasmids pGV1834 and pGV7001 respectively, wherein the SOE-PCR product and the plasmids are digested with NdeI and NcoI. The SOE-PCR product is prepared by amplifying the 5′ end of the ScADH7 gene excluding the start codon using the primers ADH7F and ADH7R. ADH7F includes 20 nucleotides of homology to the 5′ sequence of ADH3. Two oligonucleotides (ADH3MTSF, ADH3MTSR) containing the sense and the antisense strands of the coding sequence for the first 29 amino acids of Adh3p, a NdeI site, and 5 nucleotides that are homologous to the 5′ end of ADH7 are synthesized and annealed. The PCR product and the annealed oligonucleotides are combined in a SOE reaction using the primers ADH37F and ADH7R.

A NADH dependent KARI containing a mitochondrial targeting sequence (EcilvCco(Sc)^{P2D1-A1-HIS6}(SEQ ID NO: 162) which is described in co-pending U.S. application Ser. No. 12/610,784) is cloned into pGV1817 replacing ScILV5 to generate pGV1817N (SEQ ID NO: 12).

The gene DmADH gene (GI:78706922) coding for the NADH dependent Drosophila melanogaster ADH is cloned into pGV1834 replacing ADH7 to generate pGV1834N (SEQ ID NO: 14). DmADH is amplified in a PCR reaction, using primers DmADHF and 1364 and the clone RH54514 (Drosophila Genome Resource Center) as a template. The ˜0.8 kb PCR product so generated is digested with NdeI and NotI and ligated with pGV1834 which is digested with NdeI and NotI to yield pGV1834N.

The gene DmADH gene (GI:78706922) coding for the NADH dependent Drosophila melanogaster ADH is fused to the sequence coding for the Adh3p-MTS using SOE-PCR. The SOE-PCR product is prepared by amplifying the 5′ end of the DmADH7 gene excluding the start codon using the primers DmADH3F and DmADH3R. DmADH3F includes 20 nucleotides of homology to the 5′ sequence of ADH3. Two oligonucleotides (DmADH3MTSF, DmADH3MTSR) containing the sense and the antisense strands of the coding sequence for the first 29 amino acids of Adh3p, a NdeI site, and 5 nucleotides that are homologous to the 5′ end of DmADH are synthesized and annealed. The PCR product and the annealed oligonucleotides are combined in a SOE reaction using the primers ADH37F and DmADH3R. The SOE-PCR product is cloned into pGV1834 replacing ADH7 to generate pGV9834N (SEQ ID NO: 17).

The gene DmADH gene (GI:78706922) coding for the NADH dependent Drosophila melanogaster ADH is cloned into pGV7001 replacing ADH7 to generate pGV7001N (SEQ ID NO: 15). DmADH is amplified in a PCR reaction, using primers DmADHF and 1364 and the clone RH54514 (Drosophila Genome Resource Center) as a template. The ˜0.8 kb PCR product so generated is digested with NdeI and NotI and ligated with pGV7001 which is digested with NdeI and NotI to yield pGV7001N (SEQ ID NO: 15).

The gene DmADH gene (GI:78706922) coding for the NADH dependent Drosophila melanogaster ADH is fused to the sequence coding for the Adh3p-MTS using SOE-PCR. The SOE-PCR product is prepared by amplifying the 5′ end of the DmADH7 gene excluding the start codon using the primers DmADH3F and DmADH3R. DmADH3F includes 20 nucleotides of homology to the 5′ sequence of ADH3. Two oligonucleotides (DmADH3MTSF, DmADH3MTSR) containing the sense and the antisense strands of the coding sequence for the first 29 amino acids of Adh3p, a NdeI site, and 5 nucleotides that are homologous to the 5′ end of DmADH are synthesized and annealed. The PCR product and the annealed oligonucleotides are combined in a SOE reaction using the primers ADH37F and DmADH3R. The SOE-PCR product is cloned into pGV7001 replacing ADH7 to generate pGV7101N (SEQ ID NO: 16).

A NADH dependent KARI containing a mitochondrial targeting sequence is cloned into pGV1832 replacing ILV5 to generate pGV1832N (SEQ ID NO: 13).

To provide cofactor balance via malate pathway the following genes are overexpressed in the yeast host: PCK1 (GI:83722562), MDH2 (GI:84626310), DIC1 (GI:85666119) and MAE1 (GI:83722562). The genes are amplified by PCR using S. cerevisiae genomic DNA as template and cloned into an integration vector yielding pGV8000 (SEQ ID NO: 18). In this vector the PCK1 gene is controlled by the promoter P_{ScTDH3}, MDH3 is controlled by P_{ScADH1}, DIC1 is controlled by P_{ScFBA1}, and MAE1 is controlled by the promoter P_{ScTP11}. The plasmid also expresses the hygromycin resistance gene, hph under the control of the promoter P_{ScTEF1}. In addition, pGV8000 carries a sequence which consists of, from 5′ to 3′, S. cerevisiae PDC6 terminator, a unique restriction site (HpaI), and the PDC6 promoter to target the integration of the HpaI linearized plasmid to PDC6 in S. cerevisiae.

The transhydrogenase gene from Neurospora crassa (GI:164426165) (SEQ ID NO: 96) is synthesized according to the sequence from the strain N. crassa 74-OR23-1VA (FGSC#2489). The synthetic gene is flanked by EcoRI and AvrII restriction sites at the 5′ and the 3′ end of the gene respectively. The gene is cloned into the plasmid pGV9000 (SEQ ID NO: 19) in which the expression of the gene is controlled by the strong constitutive promoter TDH3. The plasmid also contains the hygromycin resistance gene, hph under the control of the promoter PScTEF1 and 3′ and 5′ targeting regions that for integration into ScPDC6. The plasmid can be linearized using the restriction site HpaI which is located between the two targeting sequences.

The plasmid pGV1816 was constructed to replace the 5′ fragment of the URA3 marker in pGV1773 with full length URA3 and a region of the PDC1 terminator downstream in order to integrate genes into the S. cerevisiae genome at the PDC1 locus and to select for integrants. The 3′ region of URA3 was PCR amplified from pGV1774 with primers 1678 and 1679, and the PDC1 terminator was amplified from pGV1774 with primers 1435 and 1680. Primer 1680 added 9 nucleotides of homology to the 3′ fragment of URA3 onto the PDC1 terminator region, and primer 1679 added 13 nucleotides of homology to the PDC1 terminator region onto the 3′ fragment of URA3. The two PCR products were combined in an SOE PCR reaction with primers 1435 and 1680. The resulting SOE PCR product and pGV1773 were digested with EcoRI and SgrAI, and the two were ligated to yield pGV1816.

The plasmid pGV1874 was constructed to replace the existing Bacillus subtilis BsalsS gene and the Lactococcus lactis LlkivD gene in pGV1816 with the same genes, but adding nucleotides encoding the 25 amino acid mitochondrial targeting sequence from the S. cerevisiae COX4 gene at the 5′ end of BsalsS and adding nucleotides encoding the mitochondrial targeting sequence from S. cerevisiae ScHMI1 at the 3′ end of LlkivD. It was constructed by PCR amplification of B. subtilis BsalsS, S. cerevisiae TDH3 promoter, and L. lactis LlkivD from pGV1773 with primers 1681 and 1661. Primer 1681 added 25 nucleotides encoding part of the first 25 amino acids of the mitochondrial targeting sequence of COX4 from S. cerevisiae to the 5′ end of BsalsS, and primer 1661 added 55 nucleotides encoding part of the mitochondrial targeting sequence of ScHMI1, to the 3′ end of LlkivD. The above PCR product was then used in an SOE PCR reaction with oligonucleotides encoding the mitochondrial targeting sequences to splice each mitochondrial targeting sequence to the appropriate end of the PCR product using primers 1683 and 1684. The oligonucleotide numbers of the mitochondrial targeting sequences are 1665 and 1666 for COX4, and 1663 and 1664 for ScHMI1. The resulting SOE PCR product was digested with NheI and XhoI, and the vector, pGV1816, was digested with SalI and AvrII, and the two were ligated to yield pGV1874.

The plasmid pGV1877 was constructed to replace the existing B. subtilis BsalsS gene in pGV1816 with BsalsS that has a 31 amino acid mitochondrial targeting sequence at its 5′ end from the S. cerevisiae COX4 gene, and the L. lactis LlkivD gene in pGV1816 with ScARO10 from S. cerevisiae that has a 3′ mitochondrial targeting sequence from the S. cerevisiae ScHMI1 gene. It was constructed by PCR amplification of B. subtilis BsalsS and S. cerevisiae TDH3 promoter from pGV1773 with primers 1682 and 1688, and S. cerevisiae ARO10 from genomic DNA with primers 1689 and 1662. Primer 1682 added 25 nucleotides encoding part of the first 31 amino acids of the targeting sequence of COX4 from S. cerevisiae to the 5′ end of BsalsS, and primer 1688 added 21 nucleotides encoding part of ARO10 to the 3′ end of the TDH3 promoter. Primer 1689 added 18 nucleotides of homology to TDH3 to the 5′ end of ARO10, and primer 1662 added 55 nucleotides encoding part of the ScHMI1 targeting sequence to the 3′ end of ARO10. The two PCR products were combined in an SOE PCR reaction with oligonucleotides encoding the targeting sequences to splice the fragments and targeting sequences using primers 1683 and 1684. The oligonucleotide numbers of the leader sequences are 1667 and 1668 for COX4, and 1663 and 1664 for ScHMI1. The resulting SOE PCR product was digested with NheI and XhoI, and the vector, pGV1816, was digested with SalI and AvrII, and the two were ligated to yield pGV1877.

The plasmid pGV1878 was constructed to replace the existing B. subtilis BsalsS gene in pGV1816 with ILV2 from S. cerevisiae, and the existing L. lactis LlkivD with Llkivd that has a 3′ mitochondrial targeting sequence from the S. cerevisiae ScHMI1 gene. It was constructed by PCR amplification of S. cerevisiae ILV2 from genomic DNA with primers 1685 and 1686, and amplification of S. cerevisiae TDH3 promoter and L. lactis LlkivD from pGV1773 with primers 1687 and 1661. Primer 1686 added 21 nucleotides encoding part of the 5′ end of the TDH3 promoter to the 3′ end of ILV2. Primer 1687 added 20 nucleotides encoding part of the 3′ end of ILV2 to the 5′ end of the TDH3 promoter, and primer 1661 added 55 nucleotides encoding part of the targeting sequence from ScHMI1 to the 3′ end of LlkivD. The two PCR products were combined in an SOE PCR reaction with the oligonucleotide of the HMI targeting sequence (1663 and 1664) to splice the fragments using primers 1685 and 1684. The resulting SOE PCR product was digested with NheI and XhoI, and the vector, pGV1816, was digested with SalI and AvrII, and the two were ligated to yield pGV1878.

The plasmid pGV1879 was constructed to replace the existing B. subtilis BsalsS gene in pGV1816 with ILV2 from S. cerevisiae, and the L. lactis LlkivD gene in pGV1816 with ARO10 from S. cerevisiae plus nucleotides encoding the mitochondrial targeting sequence from the S. cerevisiae ScHMI1 at the 3′ end of ScARO10. It was constructed by PCR amplification of S. cerevisiae ILV2 from genomic DNA with primers 1685 and 1686, S. cerevisiae ARO10 from genomic DNA with primers 1689 and 1662, and S. cerevisiae TDH3 promoter from pGV1773 with primers 1690 and 1691. Primer 1686 added 21 nucleotides encoding part of the 5′ end of the TDH3 promoter to the 3′ end of ILV2. Primer 1689 added 18 nucleotides encoding part of the 3′ end of the TDH3 promoter to the 5′ end of ARO10, and primer 1662 added 55 nucleotides encoding part of the ScHMI1 mitochondrial targeting sequence to the 3′ end of ScARO10. The three PCR products were combined in an SOE PCR reaction with the oligonucleotides encoding the ScHMI1 mitochondrial targeting sequence (1663 and 1664) to splice the fragments using primers 1685 and 1684. The resulting SOE PCR product was digested with NheI and XhoI, and the vector, pGV1816, was digested with SalI and AvrII, and the two were ligated to yield pGV1879.

The plasmid pGV1892 was constructed to replace the existing S. cerevisiae ADH7 gene in pGV1878 with derepressed full length ILV6 (ILV6*) from S. cerevisiae. It was constructed by PCR amplification of the N terminus of ILV6 from S. cerevisiae genomic DNA with primers 1740 and 1741. Primer 1740 added an NdeI site to the 5′ end of ILV6. The rest of ILV6* was PCR amplified from pGV1315 with primer 1738, which has homology to the N terminus of ILV6, and 1739 which included amplification of an existing 3′ NotI site. The resulting PCR products were combined in an SOE PCR reaction with primers 1739 and 1740. The SOE PCR product and pGV1878 were digested with Nod and NdeI, and the two were ligated to yield pGV1892.

The plasmid pGV1875 (SEQ ID NO: 92) was constructed to replace the existing Bacillus subtilis BsalsS gene and the Lactococcus lactis LlkivD gene in pGV1816 with the same genes, but adding nucleotides encoding the 31 amino acid mitochondrial targeting sequence from the S. cerevisiae COX4 gene at the 5′ end of BsalsS and adding nucleotides encoding the mitochondrial targeting sequence from S. cerevisiae Hmi1p at the 3′ end of LlkivD. It was constructed by PCR amplification of B. subtilis BsalsS, S. cerevisiae TDH3 promoter, and L. lactis LlkivD from pGV1773 with primers 1682 and 1661. Primer 1682 added 25 nucleotides part of the first 31 amino acids of the mitochondrial targeting sequence of COX4 from S. cerevisiae to the 5′ end of BsalsS, and primer 1661 added 55 nucleotides encoding part of the mitochondrial targeting sequence of Hmi1p, to the 3′ end of LlkivD. The above PCR product was then used in an SOE PCR reaction with oligonucleotides encoding the mitochondrial targeting sequences to splice each mitochondrial targeting sequence to the appropriate end of the PCR product using primers 1683 and 1684. The oligonucleotide numbers of the mitochondrial targeting sequences are 1667 and 1668 for COX4, and 1663 and 1664 for ScHMI1. The resulting SOE PCR product was digested with NheI and XhoI, and the vector, pGV1816, was digested with SalI and AvrII, and the two were ligated to yield pGV1875.

The plasmid pGV1876 (SEQ ID NO: 97) was constructed to replace the existing B. subtilis BsalsS gene in pGV1816 with BsalsS including a 25 amino acid mitochondrial targeting sequence from the S. cerevisiae COX4 gene at the 5′ end of the gene, and the L. lactis LlkivD gene in pGV1816 with ARO10 from S. cerevisiae including a mitochondrial targeting sequence from the S. cerevisiae ScHMI1 gene at the 3′ end of ARO10. It was constructed by PCR amplification of Bacillus subtilis BsalsS and S. cerevisiae TDH3 promoter from pGV1773 with primers 1681 and 1688, and S. cerevisiae ARO10 from genomic DNA with primers 1689 and 1662. Primer 1681 added 25 nucleotides of homology to the first 25 amino acids of the mitochondrial targeting sequence of subunit IV of cytochrome oxidase from S. cerevisiae to the 5′ end of BsalsS, and primer 1688 added 21 nucleotides of homology to ARO10 to the 3′ end of the TDH3 promoter. Primer 1689 added 18 nucleotides of homology to TDH3 to the 5′ end of ARO10, and primer 1662 added 55 nucleotides of homology to the ScHMI1 mitochondrial targeting sequence to the 3′ end of ARO10. The two PCR products were combined in an SOE PCR reaction with oligonucleotides encoding the mitochondrial targeting sequences to splice the fragments using primers 1683 and 1684. The oligonucleotide numbers of the mitochondrial targeting sequences are 1665 and 1666 for cytochrome oxidase, and 1663 and 1664 for ScHMI1. The resulting SOE PCR product was digested with NheI and XhoI, and the vector, pGV1816, was digested with SalI and AvrII, and the two were ligated to yield pGV1876.

Construction of pGV2212

Sc_BAT1 was cloned into pGV1104, producing pGV2212. Briefly, the sequence for Sc_BAT1 was amplified from S. cerevisiae genomic DNA using primers 2305 and 2323. These primers introduced a NotI and BamHI sites at the 5′ and 3′ ends of the coding sequence, respectively. The PCR product and pGV1104 were digested with NotI and BamHI and ligated together to produce pGV2212. The Sc_BAT1 sequence was confirmed by sequencing (Laragen, Inc., Los Angeles, Calif.).

Construction of pGV1999

pGV1441 is a derivative of p423TEF (Mumberg, D. et al. (1995) Gene 156:119-122; obtained from ATCC), where the multiple cloning sites had been changed to SalI-EcoRI-SmaI-BamHI-NotI-XhoI. pGV1999 is a high-copy HIS3 marked yeast plasmid for overexpression of the S. cerevisiae cytosolic branched chain amino acid transaminase, Bat2p. The sequence for Sc_BAT2 was amplified from S. cerevisiae genomic DNA using primers 457 and 458. These primers introduced a SalI and BamHI sites at the 5′ and 3′ ends of the coding sequence. This PCR product was digested with SalI and BamHI and cloned into the same sites of pGV1441. The Sc_BAT2 sequence was confirmed by sequencing (Laragen, Inc., Los Angeles, Calif.).

Construction of Plasmids pGV1990 and pGV2015

To generate a fragment to clone into pGV1909, Ll_kivD was PCR amplified with primers 2038 and 2039 and KOD polymerase (Novagen, catalog #71086) using pGV1590 as template. The PCR amplified DNA was purified using the Qiagen PCR Purification Kit (Qiagen kit #28106). The purified PCR DNA and pGV1909 were digested with HinDIII and NotI. The digested PCR DNA was purified by ethanol precipitation, and the appropriately sized band from pGV1909 was excised from a 1% agarose gel and purified with a Zymoclean Gel DNA Recovery Kit (Zymo Research, Orange, Calif.; Catalog #D4002). The purified insert (PCR product) and vector DNA (pGV1909) were mixed in a 6:1 molar ratio with T4 DNA Ligase and buffer (New England Biolabs, Catalog M0202), and incubated at room temperature for two hours before transformation into chemically-competent E. coli. Transformants were screened by sequence analysis. One transformant contained pGV1990. Another transformant contained a plasmid that was the result of the re-ligation of pGV1909 without Ll_kivD, which was assigned the designation pGV2015.

Construction of Plasmid pGV2061:

pGV1811 and pGV2030 were digested with SalI and NotI. The DNA was gel purified from a 1% agarose gel using the Zymoclean Gel DNA Recovery Kit (Zymo Research, Orange, Calif.; Catalog #D4002). The purified insert and vector DNA fragments were mixed in a 5:1 molar ratio with T4 DNA Ligase and buffer (New England Biolabs, Catalog M0202), and incubated at room temperature for two hours before transformation into chemically-competent E. coli. Transformants were screened by PCR, restriction digestions, and sequence analysis. The fully verified candidate was name pGV2061.

Construction of Plasmid pGV2062:

To generate a fragment to clone into pGV1812, Sc_COX4:Bs_alsS was PCR amplified with primers 2105 and 2106 and KOD polymerase (Novagen, catalog #71086) using pGV1875 as template. The PCR amplified DNA was purified using the Zymoclean and Concentrator Kit (kit #28106). The purified PCR DNA and pGV1812 were digested with BamHI and XhoI. The digested DNA was resolved on a 1% agarose gel, and the DNA fragments were excised from the gel and purified with a Zymoclean Gel DNA Recovery Kit (Zymo Research, Orange, Calif.; Catalog #D4002). The purified insert (PCR product) and vector DNA (pGV1812) were mixed in a 5:1 molar ratio with T4 DNA Ligase and buffer (New England Biolabs, Catalog M0202), and incubated at room temperature for two hours before transformation into chemically-competent E. coli. Transformants were screened by PCR, restriction digestions, and sequence analysis. The fully verified candidate was named pGV2062.

The plasmids pGV9834 (SEQ ID NO: 11), and pGV7101 (SEQ ID NO: 10), are constructed by ligating a SOE-PCR product containing nucleotides coding for the MTS of Adh3p fused to the 5′ end of the ADH7 gene into the plasmids pGV1834 (SEQ ID NO: 8), and pGV7001 (SEQ ID NO: 9), respectively, wherein the SOE-PCR product and the plasmids are digested with NdeI and NcoI. The SOE-PCR product is prepared by amplifying the 5′ end of the ADH7 gene excluding the start codon using the primers ADH7F and ADH7R. ADH7F includes 20 nucleotides of homology to the 5′ sequence of ADH3. Two oligonucleotide containing the sense and the antisense strands of the coding sequence for the first 29 amino acids of Adh3p, a NcoI site, and 5 nucleotides that are homologous to the 5′ end of the ADH7 are synthesized and annealed. The PCR product and the annealed oligonucleotides are combined in a SOE reaction using the primers ADH37 and ADH7R.

| GEVO No. | Genotype/Source |
|---|---|
| A | Kluyveromyces marxianus ura3-Δ2 pdc1Δ::[P_{KmPDC1}:25ScCOX4- |
| | MTS:BsalsS:P_{ScTDH3}:LlkivD:ScHMI1- |
| | MTS:P_{ScADH1}:SCADH7:P_{ScFBA1}:ScURA3:P_{ScFBA1}:ScILV5:P_{ScTPI1}:ScILV3:T_{KmPDC1}] |
| A1 | Kluyveromyces marxianus ura3-Δ2 pdc1Δ::[P_{KmPDC1}:25COX4- |
| | MTS:BsalsS:P_{ScTDH3}:LlkivD:ScHMI1-MTS:P_{ScADH1}: ScADH3 |
| | MTS:ScADH7:P_{ScFBA1}:ScURA3:P_{ScFBA1}:ScILV5:P_{ScTPI1}:ScILV3:T_{KmPDC1}] |
| A1MB | Kluyveromyces marxianus ura3-Δ2 pdc1Δ::[P_{KmPDC1}:25ScCOX4- |
| | MTS:BsalsS:P_{ScTDH3}:LlkivD:ScHMI1-MTS:P_{ScADH}:ScADH3- |
| | MTS:ScADH7:P_{ScFBA1}:ScURA3:P_{ScFBA1}:ScILV5:P_{ScTPI1}:ScILV3:T_{KmPDC1}] |
| | [P_{ScTDH3}:ScPCK1:P_{ScADH1}:ScMDH2:P_{ScFBA1}:ScDIC1:P_{ScTPI1}:ScMAE1:P_{ScTEF1}:hph: |
| | T_{ScPDC6}] |
| A1N | Kluyveromyces marxianus ura3-Δ2 pdc1Δ::[P_{KmPDC1}:25ScCOX4-MTS:BsalsS: |
| | P_{ScTDH3}:LlkivD:ScHMI-MTS:P_{ScADH1}:ScADH3-MTS:DmADH: P_{ScFBA1}:ScURA3: |
| | P_{ScFBA1}:31COX4-MTS:EcilvCcoSc^{P2D1-A1-his6}: P_{ScTPI1}:ScILV3: T_{KmPDC1}] |
| A1P | Kluyveromyces marxianus ura3-Δ2 [P_{ScPDC1}:25ScCOX4- |
| | MTS:BsalsS:P_{ScTDH3}:LlkivD:ScHMI1-MTS:P_{ScADH1}: ScADH3- |
| | MTS:ScADH7:P_{ScFBA1}:ScURA3:P_{ScFBA1}:ScILV5:P_{ScTPI1}:ScILV3:T_{KmPDC1}] |
| A1PMB | Kluyveromyces marxianus ura3-Δ2 [P_{ScPDC1}:25ScCOX4- |
| | MTS:BsalsS:P_{ScTDH3}:LlkivD:ScHMI1-MTS:P_{ScADH}:ScADH3- |
| | MTS:ScADH7:P_{ScFBA1}:ScURA3:P_{ScFBA1}:ScILV5:P_{ScTPI1}:ScILV3:T_{KmPDC1}] |
| | [P_{ScTDH3}:ScPCK1:P_{ScADH1}:ScMDH2:P_{ScFBA1}:ScDIC1:P_{ScTPI1}:ScMAE1:P_{ScTEF1}:hph: |
| | T_{ScPDC6}] |
| A1PN | Kluyveromyces marxianus ura3-Δ2 [P_{ScPDC1}:25ScCOX4-MTS:BsalsS: |
| | P_{ScTDH3}:LlkivD ScHMI-MTS:P_{ScADH1}: ScADH3-MTS:DmADH |
| | P_{ScFBA1}:ScURA3:P_{ScFBA1}:31COX4-MTS:EcilvCcoSc^{P2D1-A1-his6}: |
| | P_{ScTPI1}:ScILV3:T_{ScPDC1}] |
| A1PTH | Kluyveromyces marxianus ura3-Δ2 [P_{ScPDC1}:25ScCOX4- |
| | MTS:BsalsS:P_{ScTDH3}:LlkivD:ScHMI1-MTS:P_{ScADH}: ScADH3- |
| | MTS:ScADH7:P_{ScFBA1}:ScURA3:P_{ScFBA1}:ScILV5:P_{ScTPI1}:ScILV3:T_{KmPDC1}] [P_{ScTDH3}:Nc |
| | Transhydrogenase:P_{ScTEF1}:hph] |
| A1TH | Kluyveromyces marxianus ura3-Δ2 pdc1Δ::[P_{KmPDC1}:25ScCOX4- |
| | MTS:BsalsS:P_{ScTDH3}:LlkivD:ScHMI1-MTS:P_{ScADH}: ScADH3- |
| | MTS:ScADH7:P_{ScFBA1}:ScURA3:P_{ScFBA1}:ScILV5:P_{ScTPI1}:ScILV3:T_{KmPDC1}] [P_{ScTDH3}:Nc |
| | Transhydrogenase:P_{ScTEF1}:hph] |
| AK | Kluyveromyces marxianus ura3-Δ2 pdc1Δ::[P_{KmPDC1}:25ScCOX4- |
| | MTS:BsalsS:P_{ScTDH3}:LlkivD:ScHMI1- |
| | MTS:P_{ScADH1}:ScADH7:P_{ScFBA1}:ScURA3:P_{ScFBA1}:31COX4-MTS:EcilvCcoSc^{P2D1-A1-his6}: |
| | P_{ScTPI1}:ScILV3:T_{KmPDC1}] |
| AKMB | Kluyveromyces marxianus ura3-Δ2 pdc1Δ::[P_{KmPDC1}:25ScCOX4- |
| | MTS:BsalsS:P_{ScTDH3}:LlkivD:ScHMI1- |
| | MTS:P_{ScADH1}:ScADH7:P_{ScFBA1}:ScURA3:P_{ScFBA1}:31COX4-MTS:EcilvCcoSc^{P2D1-A1-his6}: |
| | P_{ScTPI1}:ScILV3:T_{KmPDC1}] |
| | [P_{ScTDH3}:ScPCK1:P_{ScADH1}:ScMDH2:P_{ScFBA1}: ScDIC1:P_{ScTPI1}:ScMAE1:P_{ScTEF1}:hph: |
| | T_{ScPDC6}] |
| AKTH | Kluyveromyces marxianus ura3-Δ2 pdc1Δ::[P_{KmPDC1}:25ScCOX4- |
| | MTS:BsalsS:P_{ScTDH3}:LlkivD:ScHMI1- |
| | MTS:P_{ScADH1}:ScADH7:P_{ScFBA1}:ScURA3:P_{ScFBA1}:31COX4-MTS:EcilvCcoSc^{P2D1-A1-his6}: |
| | P_{ScTPI1}:ScILV3:T_{KmPDC1}] [P_{ScTDH3}:Nc Transhydrogenase:P_{ScTEF1}:hph] |
| AM | Kluyveromyces marxianus ura3-Δ2 pdc1Δ::[P_{KmPDC1}:25ScCOX4- |
| | MTS:BsalsS:P_{ScTDH3}:LlkivD:ScHMI1- |
| | MTS:P_{ScADH1}:DmADH:P_{ScFBA1}:ScURA3:P_{ScFBA1}:ScILV5:P_{ScTPI1}:ScILV3:T_{KmPDC1}] |
| AMB | Kluyveromyces marxianus ura3-Δ2 pdc1Δ::[P_{KmPDC1}:25ScCOX4- |
| | MTS:BsalsS:P_{ScTDH3}:LlkivD:ScHMI1- |
| | MTS:P_{ScADH1}:ScADH7:P_{ScFBA1}:ScURA3:P_{ScFBA1}:ScILV5:P_{ScTPI1}:ScILV3:T_{KmPDC1}] |
| | [P_{ScTDH3}:ScPCK1:P_{ScADH1}:ScMDH2:P_{ScFBA1}: ScDIC1:P_{ScTPI1}:ScMAE1:P_{ScTEF1}:hph: |
| | T_{ScPDC6}] |
| AMMB | Kluyveromyces marxianus ura3-Δ2 pdc1Δ::[P_{KmPDC1}:25ScCOX4- |
| | MTS:BsalsS:P_{ScTDH3}:LlkivD:ScHMI1- |
| | MTS:P_{ScADH1}:DmADH:P_{ScFBA1}:ScURA3:P_{ScFBA1}:ScILV5:P_{ScTPI1}:ScILV3:T_{KmPDC1}] |
| | [P_{ScTDH3}:ScPCK1:P_{ScADH1}:ScMDH2:P_{ScFBA1}: ScDIC1:P_{ScTPI1}:ScMAE1:P_{ScTEF1}:hph: |
| | T_{ScPDC6}] |
| AMTH | Kluyveromyces marxianus ura3-Δ2 pdc1Δ::[P_{KmPDC1}:25ScCOX4- |
| | MTS:BsalsS:P_{ScTDH3}:LlkivD:ScHMI1- |
| | MTS:P_{ScADH1}:DmADH:P_{ScFBA1}:ScURA3:P_{ScFBA1}:ScILV5:P_{ScTPI1}:ScILV3:T_{KmPDC1}] |
| | [P_{ScTDH3}:Nc Transhydrogenase:P_{ScTEF1}:hph] |
| AN | Kluyveromyces marxianus ura3-Δ2 pdc1 Δ::[P_{KmPDC1}:25ScCOX4-MTS:BsalsS: |
| | P_{ScTDH3}:LlkivD ScHMI-MTS: P_{ScADH1}: DmADH: P_{ScFBA1}:ScURA3: |
| | P_{ScFBA1}:31COX4-MTS:EcilvCcoSc^{P2D1-A1-his6}: P_{ScTPI1}:ScILV3: T_{KmPDC1}] |
| AP | Kluyveromyces marxianus ura3-Δ2 [P_{ScPDC1}:25ScCOX4- |
| | MTS:BsalsS:P_{ScTDH3}:LlkivD:ScHMI1- |
| | MTS:P_{ScADH1}:ScADH7:P_{ScFBA1}:ScURA3:P_{ScFBA1}:ScILV5:P_{ScTPI1}:ScILV3:T_{ScPDC1}] |
| APM | Kluyveromyces marxianus ura3-Δ2 [P_{ScPDC1}:25ScCOX4- |
| | MTS:BsalsS:P_{ScTDH3}:LlkivD:ScHMI1- |
| | MTS:P_{ScADH1}:DmADH:P_{ScFBA1}:ScURA3:P_{ScFBA1}:ScILV5:P_{ScTPI1}:ScILV3:T_{ScPDC1}] |
| APMB | Kluyveromyces marxianus ura3-Δ2 [P_{ScPDC1}:25ScCOX4- |
| | MTS:BsalsS:P_{ScTDH3}:LlkivD:ScHMI1- |
| | MTS:P_{ScADH1}:ScADH7:P_{ScFBA1}:ScURA3:P_{ScFBA1}:SCILV5:P_{ScTPI1}:ScILV3:T_{ScPDC1}] |
| | [P_{ScTDH3}:ScPCK1:P_{ScADH1}:ScMDH2:P_{ScFBA1}: ScDIC1:P_{ScTPI1}:ScMAE1:P_{ScTEF1}:hph: |
| | T_{ScPDC6}] |
| APMMB | Kluyveromyces marxianus ura3-Δ2 [P_{ScPDC1}:25ScCOX4- |
| | MTS:BsalsS:P_{ScTDH3}:LlkivD:ScHMI1- |
| | MTS:P_{ScADH1}:DmADH:P_{ScFBA1}:ScURA3:P_{ScFBA1}:ScILV5:P_{ScTPI1}:ScILV3:T_{ScPDC1}] |
| | [P_{ScTDH3}:ScPCK1:P_{ScADH1}:ScMDH2:P_{ScFBA1}: ScDIC1:P_{ScTPI1}:ScMAE1:P_{ScTEF1}:hph: |
| | T_{ScPDC6}] |
| APMTH | Kluyveromyces marxianus ura3-Δ2 [P_{ScPDC1}:25ScCOX4- |
| | MTS:BsalsS:P_{ScTDH3}:LlkivD:ScHMI1- |
| | MTS:P_{ScADH1}:DmADH:P_{ScFBA1}:ScURA3:P_{ScFBA1}:ScILV5:P_{ScTPI1}:ScILV3:T_{ScPDC1}] |
| | [P_{ScTDH3}:Nc Transhydrogenase:P_{ScTEF1}:hph] |
| APN | Kluyveromyces marxianus ura3-Δ2 [P_{ScPDC1}:25ScCOX4- |
| | MTS:BsalsS:P_{ScTDH3}:LlkivD:ScHMI1- |
| | MTS:P_{ScADH1}:DmADH:P_{ScFBA1}:ScURA3:P_{ScFBA1}:31COX4- |
| | MTS:EcilvCcoSc^{P2D1-A1-his6}:P_{ScTPI1}:ScILV3:T_{ScPDC1}] |
| APTH | Kluyveromyces marxianus ura3-Δ2 [P_{ScPDC1}:25ScCOX4- |
| | MTS:BsalsS:P_{ScTDH3}:LlkivD:ScHMI1- |
| | MTS:P_{ScADH1}:ScADH7:P_{ScFBA1}:ScURA3:P_{ScFBA1}:SCILV5:P_{ScTPI1}:ScILV3:T_{ScPDC1}] |
| | [P_{ScTDH3}:Nc Transhydrogenase:P_{ScTEF1}:hph] |
| ATH | Kluyveromyces marxianus ura3-Δ2 pdc1Δ::[P_{KmPDC1}:25ScCOX4- |
| | MTS:BsalsS:P_{ScTDH3}:LlkivD:HMI1- |
| | MTS:P_{ScADH1}:ScADH7:P_{ScFBA1}:ScURA3:P_{ScFBA1}:ScILV5:P_{ScTPI1}:ScILV3:T_{KmPDC1}] |
| | [P_{ScTDH3}:Nc Transhydrogenase:P_{ScTEF1}:hph] |
| c2i-SC1 | S. cerevisiae MATa his3 trp1 ura3 pdc5::ble pdc6::apt1(kanR) ho |
| | pdc1Δ::(P_{ScPDC1}:25ScCOX4-MTS:BsalsS:P_{ScTDH3}:LlkivD:ScHMI1- |
| | MTS:P_{ScADH1}:ScADH7:P_{ScFBA1}:ScURA3:P_{ScFBA1}:ScILV5:P_{ScTPI1}:ScILV3:T_{ScPDC1}) |
| | Evolved to be C2-independent |
| c2i-SC1M | S. cerevisiae MATa his3 trp1 ura3 pdc5::ble pdc6::apt1(kanR) ho |
| | pdc1Δ::[P_{ScPDC1}:25ScCOX4-MTS:BsalsS:P_{ScTDH3}:LlkivD:ScHMI1- |
| | MTS:P_{ScADH1}:DmADH:P_{ScFBA1}:ScURA3:P_{ScFBA1}:ScILV5:P_{ScTPI1}:ScILV3:T_{ScPDC1}] |
| | Evolved to be C2-independent |
| c2i-SC1MB | S. cerevisiae MATa his3 trp1 ura3 pdc5::ble ho pdc1Δ::[P_{ScPDC1}:25ScCOX4- |
| | MTS:BsalsS:P_{ScTDH3}:LlkivD:ScHMI1- |
| | MTS:P_{ScADH1}:ScADH7:P_{ScFBA1}:ScURA3:P_{ScFBA1}:ScILV5:P_{ScTPI1}:ScILV3:T_{ScPDC1}) |
| | pdc6Δ::[P_{ScTDH3}:ScPCK1:P_{ScADH1}:ScMDH2:P_{ScFBA1}:ScDIC1:P_{ScTPI1}:ScMAE1: |
| | P_{ScTEF1}:hph:T_{ScPDC6}] Evolved to be C2-independent |
| c2i-SC1MMB | S. cerevisiae MATa his3 trp1 ura3 pdc5::ble pdc6::apt1(kanR) ho |
| | pdc1Δ::[P_{ScPDC1}:25ScCOX4-MTS:BsalsS:P_{ScTDH3}:LlkivD:ScHMI1- |
| | MTS:P_{ScADH1}:DmADH:P_{ScFBA1}:ScURA3:P_{ScFBA1}:ScILV5:P_{ScTPI1}:ScILV3:T_{ScPDC1}) |
| | pdc6 Δ::[P_{ScTDH3}:ScPCK1:P_{ScADH1}:ScMDH2:P_{ScFBA1}:ScDIC1:P_{ScTPI1}:ScMAE1: |
| | P_{ScTEF1}:hph:T_{ScPDC6}] Evolved to be C2-independent |
| c2i-SC1MTH | S. cerevisiae MATa his3 trp1 ura3 pdc5::ble pdc6::apt1(kanR) ho |
| | pdc1Δ::[P_{ScPDC1}:25ScCOX4-MTS:BsalsS:P_{ScTDH3}:LlkivD:ScHMI1- |
| | MTS:P_{ScADH1}:DmADH:P_{ScFBA1}:ScURA3:P_{ScFBA1}:ScILV5:P_{ScTPI1}:ScILV3:T_{ScPDC1}] |
| | pdc6 Δ::[P_{ScTDH3}: Nc Transhydrogenase:P_{ScTEF1}:hph] Evolved to be C2- |
| | independent |
| c2i-SC1N | S. cerevisiae MATa his3 trp1 ura3 pdc5::ble pdc6::apt1(kanR) ho |
| | pdc1Δ::[P_{PDC1}:25COX-MTS:BsalsS:P_{TDH3}:LlkivD:ScHMI1- |
| | MTS:P_{ScADH1}:DmADH:P_{FBA1}:URA3:P_{FBA1}:31COX4-MTS:EcilvCcoSc^{P2D1-A1-his6}: |
| | P_{TPI1}:ILV3:T_{PDC1}] Evolved to be C2-independent |
| c2i-SC1TH | S. cerevisiae MATa his3 trp1 ura3 pdc5::ble ho pdc1Δ::[P_{ScPDC1}:25ScCOX4- |
| | MTS:BsalsS:P_{ScTDH3}:LlkivD:ScHMI1- |
| | MTS:P_{ScADH1}:ScADH7:P_{ScFBA1}:ScURA3:P_{ScFBA1}:ScILV5:P_{ScTPI1}:ScILV3:T_{ScPDC1} |
| | [pdc6 Δ::[P_{ScTDH3}: Nc Transhydrogenase:P_{ScTEF1}:hph] Evolved to be C2- |
| | independent |
| c2i-SCA1 | S. cerevisiae MATa his3 trp1 ura3 pdc5::ble pdc6::apt1(kanR) ho |
| | pdc1Δ::[P_{ScPDC1}:25ScCOX4-MTS:BsalsS:P_{ScTDH3}:LlkivD:ScHMI1- |
| | MTS:P_{ScADH1}:ScADH3- |
| | MTS:ScADH7:P_{ScFBA1}:ScURA3:P_{ScFBA1}:ScILV5:P_{ScTPI1}:ScILV3:T_{ScPDC1}] Evolved to |
| | be C2-independent |
| c2i-SCA1MB | S. cerevisiae MATa his3 trp1 ura3 pdc5::ble pdc6::apt1(kanR) ho |
| | pdc1Δ::[P_{ScPDC1}:25ScCOX4-MTS:BsalsS:P_{ScTDH3}:LlkivD:ScHMI1- |
| | MTS:P_{ScADH1}:ScADH3- |
| | MTS:ScADH7:P_{ScFBA1}:ScURA3:P_{ScFBA1}:ScILV5:P_{ScTPI1}:ScILV3:T_{ScPDC1}] |
| | pdc6Δ::[P_{ScTDH3}:ScPCK1:P_{ScADH1}:ScMDH2:P_{ScFBA1}:ScDIC1:P_{ScTPI1}:ScMAE1: |
| | P_{ScTEF1}:hph:T_{ScPDC6}] Evolved to be C2-independent |
| c2i-SCA1N | S. cerevisiae MATa his3 trp1 ura3 pdc5::ble pdc6::apt1(kanR) ho |
| | pdc1Δ::[P_{PDC1}:25COX4-MTS:BsalsS:P_{TDH3}:LlkivD:ScHMI1- |
| | MTS:P_{ScADH1}:ScADH3-MTS:DmADH:P_{ScFBA1}:ScURA3: P_{FBA1}:31COX4- |
| | MTS:EcilvCcoSc^{P2D1-A1-his6}:P_{ScTPI1}:ScILV3: T_{PDC1}] Evolved to be C2-independent |
| c2i-SCA1TH | S. cerevisiae MATa his3 trp1 ura3 pdc5::ble pdc6::apt1(kanR) ho |
| | pdc1Δ::[P_{ScPDC1}:25ScCOX4-MTS:BsalsS:P_{ScTDH3}:LlkivD:ScHMI1- |
| | MTS:P_{ScADH1}:ScADH3- |
| | MTS:ScADH7:P_{ScFBA1}:ScURA3:P_{ScFBA1}:ScILV5:P_{ScTPI1}:ScILV3:T_{ScPDC1}] |
| | pdc6Δ::[P_{ScTDH3}:Nc Transhydrogenase:P_{ScTEF1}:hph]vEvolved to be C2-independent |
| GEVO1068 | Kluyveromyces marxianus (NRRL-Y-7571 from USDA) |
| GEVO1186 | S. cerevisiae CEN.PK, ura3/ura3 leu2/leu2 his3/his3 trp1/trp1 ADE2 MAT a/alpha |
| GEVO1187 | Saccharomyces cerevisiae (CEN.PK2-1C) |
| | MATa ura3 leu2 his3 trp1 ADE2 |
| GEVO1537 | Saccharomyces cerevisiae (GG570 from Prof. Paul can Heusden, Univ. of Leiden, |
| | Netherlands) MATa/MATalpha HIS3/HIS3 LEU2/LEU2 TRP1/TRP1 URA3/URA3 |
| | pdc1::ble/pdc1::ble pdc5::ble/pdc5::ble pdc6::apt1(kanR)/pdc6::apt1(kanR) |
| | HO/HO |
| GEVO1584 | S. cerevisiae MAT a his3 trp1 ura3 leu2 pdc1::ble pdc5::ble pdc6::apt1(kanR) ho |
| GEVO1802 | S. cerevisiae CEN.PK MATa/alpha ura3/ura3 leu2/leu2 his3/his3 trp1/trp1 |
| | pdc6Δ::[ScURA3:ampR:PMB1:P_{ScTEF1}:LlkivD2:P_{ScTDH3}:ScADH7: T_{ScPDC6}]/PDC6 |
| GEVO1803 | MATa/α ura3/ura3 leu2/leu2 his3/his3 trp1/trp1 pdc1Δ::[ScTRP1:bla:pUC |
| | ori:P_{ScCUP1-1}:Bs_alsS2::T_{Sc}_{_{—}}_{PDC1}]/PDC1 |
| GEVO1805 | S. cerevisiae CEN.PK MATa/alpha ura3/ura3 leu2/leu2 his3/his3 trp1/trp1 |
| | pdc5Δ::[ScLEU2:ampR:PMB1:P_{ScTEF1}:EcilvDΔNcoKl:P_{ScTDH3}:EcilvCΔN: |
| | T_{ScPDC5}]/PDC5 pdc6Δ::[ScURA3:ampR:PMB1:P_{ScTEF1}:LlkivD2:P_{ScTDH3}:ScADH7: |
| | T_{ScPDC6}]/PDC6 |
| GEVO1820 | S. cerevisiae CEN.PK MAT a/alpha ura3/ura3 leu2/leu2 his3/his3 trp1/trp1 |
| | pdc1Δ::[ScTRP1:bla:pUC ori:P_{ScCUP1-1}:Bs_alsS2::T_{Sc}_{_{—}}_{PDC1}]/PDC1 pdc5 |
| | Δ::[ScLEU2:ampR:PMB1:P_{ScTEF1}:EcilvDΔNcoKl:P_{ScTDH3}: EcilvCΔN: T_{ScPDC5}]/ |
| | PDC5 pdc6 Δ::[ScURA3:ampR:PMB1:P_{ScTEF1}: LlkivD2:P_{ScTDH3}:ScADH7:T_{ScPDC6}]/ |
| | PDC6 |
| GEVO1863 | Saccharomyces cerevisiae MAT a his3 trp1 ura3 pdc1::ble pdc5::ble |
| | pdc6::apt1(kanR) ho. Evolved to be C2-independent |
| GEVO1947 | Kluyveromyces marxianus ura3-Δ2 |
| GEVO1969 | K. marxianus NRRL-Y-7571 ura3-Δ2 pdc1Δ::G418^{R} |
| GEVO2062 | S. cerevisiae CEN.PK MAT a/alpha ura3/ura3 leu2/leu2 his3/his3 trp1/trp1 |
| | ho/ho, pdc1Δ::[P_{ScPDC1}25COX4-MTS:Bs_alsSP_{ScTDH3}:Sc_ARO10:ScHMI1- |
| | MTS:P_{ScADH1}:Sc_ADH7:P_{Sc}FBA1:ScURA3] |
| GEVO2072 | S. cerevisiae CEN.PK MAT a/alpha ura3/ura3 leu2/leu2 his3/his3 trp1/trp1 |
| | ho/ho, PDC1/pdc1::[P_{ScPDC1}:31COX4-MTS:Bs_alsS:P_{ScTDH3}:Ll_kivD:ScHMI1- |
| | MTS:P_{ScADH1}:Sc_ADH7:P_{Sc}_{_{—}}_{FBA1}:ScURA3] |
| GEVO2087 | Kluyveromyces marxianus, ura3-Δ2 [P_{ScPDC1}:31COX4- |
| | MTS:BsalsS:P_{ScTDH3}:LlkivD:ScHMI1-MTS:P_{ScADH1}:ScADH7:PScFBA1:URA3] |
| GEVO2088 | Kluyveromyces marxianus, ura3-Δ2 [P_{ScPDC1}:31COX4- |
| | MTS:Bs_alsS:P_{ScTDH3}:LlkivD:ScHMI1-MTS:P_{ScADH1}:ScADH7:PSc_{FBA1}:URA3] |
| GEVO2107 | MATa/α ura3/ura3 leu2/leu2 his3/his3 trp1/trp1 pdc1Δ::[Bs_alsS2; TRP1]/PDC1 |
| | pdc6Δ::[P_{ScTEF1}:Llkivd:P_{ScTDH3}:DmADH ScURA3]/PDC6 |
| GEVO2119 | Saccharomyces cerevisiae diploid of CEN.PK parent strain, MAT-a/alpha |
| | ura3/ura3 leu2/leu2 his3/his3 trp1/trp1 ho/ho, pdc1Δ::[P_{ScPDC1}:25COX4- |
| | MTS:Bs_alsS:P_{ScTDH3}:LlkivD:HMI1-MTS:P_{ScADH1}:ScADH7: P_{ScFBA1}:ScURA3]/ |
| | PDC1 |
| GEVO2120/ | Saccharomyces cerevisiae diploid of CEN.PK parent strain, MAT-a/alpha |
| 2121 | ura3/ura3 leu2/leu2 his3/his3 trp1/trp1 ho/ho, pdc1Δ::[P_{ScPDC1}:31COX4- |
| | MTS:Bs_alsS:P_{ScTDH3}:ARO10:ScHMI1-MTS:P_{ScADH1}:ScADH7: |
| | P_{ScFBA1}:ScURA3]/PDC1 |
| GEVO2122/ | Saccharomyces cerevisiae diploid of CEN.PK parent strain, MAT-a/alpha |
| 2123 | ura3/ura3 leu2/leu2 his3/his3 trp1/trp1 ho/ho, pdc1Δ::[P_{ScPDC1}:ScILV2: |
| | P_{ScTDH3}:Ll_kivd:ScHMI1-MTS:P_{ScADH1}:ScADH7:P_{ScFBA1}:ScURA3]/PDC1 |
| GEVO2124/ | Saccharomyces cerevisiae diploid of CEN.PK parent strain, MAT-a/alpha |
| 2125 | ura3/ura3 leu2/leu2 his3/his3 trp1/trp1 ho/ho, pdc1Δ::[P_{ScPDC1}:ScILV2: |
| | P_{ScTDH3}:ScARO10:ScHMI1-MTS:P_{ScADH1}:ScADH7:P_{ScFBA1}:ScURA3]/PDC1 |
| GEVO2126 | Saccharomyces cerevisiae diploid of CEN.PK parent strain, MAT-a/alpha |
| | ura3/ura3 leu2/leu2 his3/his3 trp1/trp1 ho/ho, pdc1Δ::[P_{ScPDC1}:ScILV2: |
| | P_{ScTDH3}:Ll_kivd:ScHMI1-MTS:P_{ScADH1}:ScILV6*:P_{ScFBA1}:ScURA3]/PDC1 |
| GEVO2127/ | Saccharomyces cerevisiae diploid of CEN.PK parent strain, MATa/alpha |
| 2128 | ura3/ura3 leu2/leu2 his3/his3 trp1/trp1 ho/ho, PDC1/pdc1Δ::[P_{Sc}_{_{—}}_{PDC1}: 31COX4- |
| | MTS:Bs_alsS:P_{Sc}_{_{—}}_{ADH1}:Sc_ADH7:P_{Sc}_{_{—}}_{FBA1}:ScURA3] |
| GEVO2129/2130 | Saccharomyces cerevisiae diploid of CEN.PK parent strain, MATa/alpha |
| | ura3/ura3 leu2/leu2 his3/his3 trp1/trp1 ho/ho, PDC1/pdc1 Δ::[P_{Sc}_{_{—}}_{PDC1}: 31COX4- |
| | MTS:Bs_alsS:P_{Sc}_{_{—}}_{TDH3}:LlkivD:ScHMI1-MTS:P_{Sc}_{_{—}}_{FBA1}: ScURA3] |
| GEVO2131/2132 | Saccharomyces cerevisiae diploid of CEN.PK parent strain, MATa/alpha |
| | ura3/ura3 leu2/leu2 his3/his3 trp1/trp1 ho/ho, PDC1/pdc1Δ::[P_{Sc}_{_{—}}_{PDC1}: |
| | LlkivD:ScHMI1-MTS:P_{Sc}_{_{—}}_{ADH1}:ScADH7:P_{Sc}_{_{—}}_{FBA1}:ScURA3] |
| GEVO2158 | MATa/α ura3/ura3 leu2/leu2 his3/his3 trp1/trp1 pdc1 Δ::[BsalsS2: TRP1]/PDC1 |
| | pdc5 Δ::[P_{ScTEF1}:ILV3ΔN:P_{ScTDH3}:ilvCco(Sc)^{Q110V}:LEU2]/PDC5 |
| | pdc6Δ::[P_{ScTEF1}:LlkivD:P_{ScTDH3}:DmADH:ScURA3]/PDC6 |
| GEVO2166 | S. cerevisiae MATa his3 trp1 ura3 leu2 pdc1Δ::[P_{ScPDC1}:25ScCOX4- |
| | MTS:BsalsS:P_{ScTDH3}:LlkivD:ScHMI1-MTS:P_{ScADH1}:ScADH7:P_{ScFBA1}:ScURA3] |
| | pdc5::ble pdc6::apt1(kanR) ho |
| GEVO2167 | S. cerevisiae MATa his3 trp1 ura3 leu2 pdc1Δ::[P_{ScPDC1}:25ScCOX4- |
| | MTS:BsalsS:P_{ScTDH3}:LlkivD: ScHMI1-MTS:P_{ScADH1}:ScADH7:P_{ScFBA1}:ScURA3] |
| | pdc5::ble pdc6::apt1(kanR) |
| GEVO2276 | K. marxianus NRRL-Y-7571 ura3-Δ2 pdc1Δ::[P_{ScPDC1}:31ScCOX4- |
| | MTS:BsalsS:P_{ScTDH3}:LlkivD:ScHMI1- |
| | MTS:P_{ScADH1}:ScADH7:P_{ScFBA1}:ScURA3:T_{ScPDC1}] RANDOMLY INTEGRATED, |
| | Candidate #1 |
| GEVO2277 | K. marxianus NRRL-Y-7571 ura3-Δ2 pdc1Δ::[P_{ScPDC1}:31ScCOX4- |
| | MTS:BsalsS:P_{ScTDH3}:LlkivD:ScHMI1-MTS:P_{ScADH1}:ScADH7:P_{ScFBA1}:ScURA3: |
| | T_{ScPDC1}] RANDOMLY INTEGRATED Candidate #2 |
| GEVO2302 | S. cerevisiae haploid of CEN.PK parent strain MATa ura3 leu2 his3 trp1 |
| | pdc1Δ::[BsalsS2:TRP1] pdc5Δ::[P_{TEF1}:ScILV3ΔN P_{TDH3}:EcilvCco(Sc)^{Q110V}:LEU2] |
| | pdc6Δ::[P_{TEF1}:Ll_kivd2:P_{TDH3}:DmADH: URA3] |
| GEVO2346 | K. marxianus NRRL-Y-7571 ura3-Δ2 pdc1Δ::G418^{R} [P_{ScPDC1}:31ScCOX4- |
| | MTS:BsalsS:P_{ScTDH3}:LlkivD2:P_{ScFBA1}:ScURA3:T_{ScPDC1}] RANDOMLY |
| | INTEGRATED Candidate pGV1990 #4 |
| GEVO2347 | K. marxianus NRRL-Y-7571 ura3-Δ2 pdc1Δ::G418^{R} [P_{ScPDC1}:31ScCOX4-MTS: |
| | BsalsS: P_{ScTDH3}:LlkivD2:P_{ScFBA1}:ScURA3:T_{ScPDC1}] RANDOMLY INTEGRATED |
| | Candidate pGV1990 #5 |
| GEVO2348 | K. marxianus NRRL-Y-7571 ura3-Δ2 pdc1Δ::G418^{R} [P_{ScPDC1}:31ScCOX4- |
| | MTS:BsalsS:P_{ScTDH3}:P_{ScFBA1}:ScURA3:T_{ScPDC1}] RANDOMLY INTEGRATED |
| | Candidate pGV2015 #2 |
| GEVO2542 | K. marxianus NRRL-Y-7571 ura3-Δ2 pdc1Δ::[LlkivD2:P_{ScTDH3}: |
| | DmADH:P_{ScFBA1}:31ScCOX4-MTS:Bs_alsS] |
| GEVO2878 | S. cerevisiae CEN.PK MAT a/alpha ura3/ura3 leu2/leu2 his3/his3 trp1/trp1 |
| | ho/ho, pdc1Δ::[P_{ScPDC1}31COX4-MTS:BsalsS:P_{ScTDH3}: LlkivD:ScHMI1-MTS:P_{ScADH1} |
| | ScADH7:P_{ScFBA1}URA3] [P_{ScTDH3}ScBAT1, 2μ, TRP1] [P_{ScTEF1}ScBAT2, 2μ, |
| | HIS3] |
| GEVO2879 | S. cerevisiae CEN.PK MAT a/alpha ura3/ura3 leu2/leu2 his3/his3 trp1/trp1 |
| | ho/ho, pdc1Δ::[P_{ScPDC1}31COX4-MTS:BsalsS: P_{ScTDH3}:LlkivD:ScHMI1- |
| | MTS:P_{ScADH1}ScADH7:P_{ScFBA1}URA3] [P_{ScTDH3}ScBAT1, 2μ, TRP1] [P_{ScTEF1}Sc_BAT2, |
| | 2μ, HIS3] |
| GEVO2880 | S. cerevisiae CEN.PK MAT a/alpha ura3/ura3 leu2/leu2 his3/his3 trp1/trp1 |
| | ho/ho, pdc1Δ::[P_{ScPDC1}31COX4-MTS:BsalsS_co_Sc:P_{ScTDH3}:LlkivD: ScHMI1- |
| | MTS: P_{ScADH1}Sc_ADH7:P_{ScFBA1}URA3] [P_{ScTDH3}Sc_BAT1, 2μ, TRP1] |
| | [P_{ScTEF1}Sc_BAT2, 2μ, HIS3] |
| GEVO7777 | S. cerevisiae MATa his3 trp1 ura3 pdc5::ble pdc6::apt1(kanR) ho |
| SC1 | S. cerevisiae MATa his3 trp1 ura3 pdc5::ble pdc6::apt1(kanR) ho |
| | pdc1Δ::[P_{ScPDC1}:25ScCOX4-MTS:BsalsS:P_{ScTDH3}::LlkivD:ScHMI1- |
| | MTS:P_{ScADH1}:ScADH7:P_{ScFBA1}:ScURA3:P_{ScFBA1}:ScILV5: P_{ScTPI1}:ScILV3:T_{ScPDC1}] |
| SC1M | S. cerevisiae MATa his3 trp1 ura3 pdc5::ble pdc6::apt1(kanR) ho |
| | pdc1Δ::[P_{ScPDC1}:25ScCOX4-MTS:BsalsS:P_{ScTDH3}:LlkivD:ScHMI1- |
| | MTS:P_{ScADH1}:DmADH:P_{ScFBA1}:ScURA3:P_{ScFBA1}:ScILV5:P_{ScTPI1}:ScILV3:T_{ScPDC1}] |
| SC1MB | S. cerevisiae MATa his3 trp1 ura3 pdc5::ble ho |
| | pdc1Δ::[P_{ScPDC1}:25ScCOX4:BsalsS:P_{ScTDH3}:LlkivD:ScHMI1- |
| | MTS:P_{ScADH1}:ScADH7:P_{ScFBA1}:ScURA3:P_{ScFBA1}:ScILV5:P_{ScTPI1}:ScILV3:T_{ScPDC1}] |
| | pdc6Δ::[P_{ScTDH3}:ScPCK1:P_{ScADH1}:ScMDH2:P_{ScFBA1}:ScDIC1:P_{ScTPI1}:ScMAE1: |
| | P_{ScTEF1}:hph:T_{ScPDC6}] |
| SC1MMB | S. cerevisiae MATa his3 trp1 ura3 pdc5::ble pdc6::apt1(kanR) ho |
| | pdc1Δ::[P_{ScPDC1}:25ScCOX4-MTS:BsalsS:P_{ScTDH3}:LlkivD:ScHMI1- |
| | MTS:P_{ScADH1}:DmADH:P_{ScFBA1}:ScURA3:P_{ScFBA1}:ScILV5:P_{ScTPI1}:ScILV3:T_{ScPDC1}] |
| | pdc6Δ::[P_{ScTDH3}:ScPCK1:P_{ScADH1}:ScMDH2:P_{ScFBA1}:ScDIC1:P_{ScTPI1}:ScMAE1: |
| | P_{ScTEF1}:hph:T_{ScPDC6}] |
| SC1MTH | S. cerevisiae MATa his3 trp1 ura3 pdc5::ble (kanR) ho |
| | pdc1Δ::[P_{ScPDC1}:25ScCOX4-MTS:BsalsS:P_{ScTDH3}:LlkivD:ScHMI1- |
| | MTS:P_{ScADH1}:DmADH:P_{ScFBA1}:ScURA3:P_{ScFBA1}:ScILV5:P_{ScTPI1}:ScILV3:T_{ScPDC1}] |
| | pdc6Δ::[P_{ScTDH3}:Nc Transhydrogenase:P_{ScTEF1}:hph] |
| SC1N | S. cerevisiae MATa his3 trp1 ura3 pdc5::ble pdc6::apt1(kanR) ho |
| | pdc1Δ::[P_{PDC1}:25COX4-MTS:BsalsS:P_{TDH3}:LlkivD:ScHMI1-MTS: |
| | P_{ScADH1}:DmADH: P_{ScFBA1}:URA3: P_{ScFBA1}:31COX4-MTS:EcilvCcoSc^{P2D1-A1-his6}: |
| | P_{ScTPI1}:ILV3: T_{PDC1}] |
| SC1TH | S. cerevisiae MATa his3 trp1 ura3 pdc5::ble ho pdc1Δ::[P_{ScPDC1}:25ScCOX4- |
| | MTS:BsalsS:P_{ScTDH3}:LlkivD:ScHMI1- |
| | MTS:P_{ScADH1}:ScADH7:P_{ScFBA1}:ScURA3:P_{ScFBA1}:ScILV5:P_{ScTPI1}:ScILV3: T_{ScPDC1}] |
| | pdc6Δ::[P_{ScTDH3}:Nc Transhydrogenase:P_{ScTEF1}:hph] |
| SCA1 | S. cerevisiae MATa his3 trp1 ura3 pdc5::ble pdc6::apt1(kanR) ho |
| | pdc1Δ::[P_{ScADH1}:25ScCOX4-MTS:BsalsS:P_{ScTDH3}:LlkivD:ScHMI1- |
| | MTS:P_{ScADH1}:ScADH3- |
| | MTS:ScADH7:P_{ScFBA1}:ScURA3:P_{ScFBA1}:ScILV5:P_{ScTPI1}:ScILV3:T_{ScPDC1}] |
| SCA1MB | S. cerevisiae MATa his3 trp1 ura3 pdc5::ble pdc6::apt1(kanR) ho |
| | pdc1Δ::[P_{ScPDC1}:25ScCOX4-MTS:BsalsS:P_{ScTDH3}:LlkivD:ScHMI1- |
| | MTS:P_{ScADH}:ScADH3-MTS: |
| | ScADH7:P_{ScFBA1}:ScURA3:P_{ScFBA1}:ScILV5:P_{ScTPI1}:ScILV3:T_{ScPDC1}] |
| | pdc6Δ::[P_{ScTDH3}:ScPCK1:P_{ScADH1}:ScMDH2:P_{ScFBA1}:ScDIC1:P_{ScTPI1}:ScMAE1: |
| | P_{ScTEF1}:hph:T_{ScPDC6}] |
| SCA1N | S. cerevisiae MATa his3 trp1 ura3 pdc5::ble pdc6::apt1(kanR) ho |
| | pdc1Δ::[P_{PDC1}:25COX4-MTS:BsalsS:P_{TDH3}:LlkivD:ScHMI1- |
| | MTS:P_{ScADH1}:ScADH3-MTS:DmADH: P_{ScFBA1}:ScURA3: P_{ScFBA1}:31COX4- |
| | MTS:EcilvCcoSc^{P2D1-A1-his6}:P_{ScTPI1}:ScILV3:T_{PDC1}] |
| SCA1TH | S. cerevisiae MATa his3 trp1 ura3 pdc5::ble pdc6::apt1(kanR) ho |
| | pdc1Δ::[P_{ScPDC1}:25ScCOX4-MTS:BsalsS:P_{ScTDH3}:LlkivD:ScHMI1- |
| | MTS:P_{ScADH}:ScADH3-MTS: |
| | ScADH7:P_{ScFBA1}:ScURA3:P_{ScFBA1}:ScILV5:P_{ScTPI1}:ScILV3:T_{ScPDC1}] |
| | pdc6Δ::[P_{ScTDH3}:Nc Transhydrogenase:P_{ScTEF1}:hph] |
| SCP1 | S. cerevisiae MAT-a/alpha ura3/ura3 leu2/leu2 his3/his3 trp1/trp1 ho/ho |
| | pdc1Δ:: P_{ScPDC1}:25ScCOX4-MTS:BsalsS:P_{ScTDH3}:LlkivD:ScHMI1- |
| | MTS:P_{ScADH1}:ScADH7:P_{ScFBA1}:ScURA3:P_{ScFBA1}:ScILV5:P_{ScTPI1}:ScILV3:T_{ScPDC1}]/PDC1 |
| SCP1K | S. cerevisiae MAT-a/alpha ura3/ura3 leu2/leu2 his3/his3 trp1/trp1 ho/ho |
| | pdc1Δ::[P_{ScPDC1}:25ScCOX4-MTS:BsalsS:P_{ScTDH3}:LlkivD:ScHMI1- |
| | MTS:P_{ScADH1}:ScADH7:P_{ScFBA1}:ScURA3:P_{ScFBA1}:31COX4-MTS:EcilvCcoSc^{P2D1-A1-his6}: |
| | P_{ScTPI1}:ScILV3:T_{ScPDC1}]/PDC1 |
| SCP1KMB | S. cerevisiae MAT-a/alpha ura3/ura3 leu2/leu2 his3/his3 trp1/trp1 ho/ho pdc1Δ:: |
| | P_{ScPDC1}:25ScCOX4-MTS:BsalsS:P_{ScTDH3}:LlkivD:ScHMI1- |
| | MTS:P_{ScADH1}:ScADH7:P_{ScFBA1}:ScURA3:P_{ScFBA1}:31COX4-MTS:EcilvCcoSc^{P2D1-A1-his6}: |
| | P_{ScTPI1}:ScILV3:T_{ScPDC1}]/PDC1 |
| | pdc6Δ::[P_{ScTDH3}:ScPCK1:P_{ScADH1}:ScMDH2:P_{ScFBA1}:ScDIC1:P_{ScTPI1}:ScMAE1: |
| | P_{ScTEF1}:hph:T_{ScPDC6}]/PDC6 |
| SCP1KTH | S. cerevisiae MAT-a/alpha ura3/ura3 leu2/leu2 his3/his3 trp1/trp1 ho/ho |
| | pdc1Δ:[P_{ScPDC1}:25ScCOX4-MTS:BsalsS:P_{ScTDH3}:LlkivD:ScHMI1- |
| | MTS:P_{ScADH1}:ScADH7:P_{ScFBA1}:ScURA3:P_{ScFBA1}:31COX4-MTS:EcilvCcoSc^{P2D1-A1-his6}: |
| | P_{ScTPI1}:ScILV3:T_{ScPDC1}]/PDC1 pdc6Δ::[P_{ScTDH3}:Nc |
| | Transhydrogenase:P_{ScTEF1}:hph:]/PDC6 |
| SCP1M | S. cerevisiae MAT-a/alpha ura3/ura3 leu2/leu2 his3/his3 trp1/trp1 ho/ho |
| | pdc1Δ::[P_{ScPDC1}:25ScCOX4-MTS:BsalsS:P_{ScTDH3}:LlkivD:ScHMI1- |
| | MTS:P_{ScADH1}:DmADH:P_{ScFBA1}:ScURA3:P_{ScFBA1}:ScILV5:P_{ScTPI1}:ScILV3:T_{ScPDC1}]/PDC1 |
| SCP1MB | S. cerevisiae MAT-a/alpha ura3/ura3 leu2/leu2 his3/his3 trp1/trp1 ho/ho |
| | pdc1Δ::[P_{ScPDC1}:25ScCOX4-MTS:BsalsS:P_{ScTDH3}:LlkivD:ScHMI1- |
| | MTS:P_{ScADH1}:ScADH7:P_{ScFBA1}:ScURA3:P_{ScFBA1}:SCILV5:P_{ScTPI1}:ScILV3:T_{ScPDC1}]/PDC1 |
| | pdc6Δ::[P_{ScTDH3}:ScPCK1:P_{ScADH1}:ScMDH2:P_{ScFBA1}:ScDIC1:P_{ScTPI1}:ScMAE1: |
| | P_{ScTEF1}:hph:T_{ScPDC6}]/PDC6 |
| SCP1MMB | S. cerevisiae MAT-a/alpha ura3/ura3 leu2/leu2 his3/his3 trp1/trp1 ho/ho |
| | pdc1Δ::[P_{ScPDC1}:25ScCOX4-MTS:BsalsS:P_{ScTDH3}:LlkivD:ScHMI1- |
| | MTS:P_{ScADH1}:DmADH:P_{ScFBA1}:ScURA3:P_{ScFBA1}:ScILV5:P_{ScTPI1}:ScILV3:T_{ScPDC1}]/PDC1 |
| | pdc6Δ::[P_{ScTDH3}:ScPCK1:P_{ScADH1}:ScMDH2:P_{ScFBA1}:ScDIC1:P_{ScTPI1}:ScMAE1: |
| | P_{ScTEF1}:hph:T_{ScPDC6}]/PDC6 |
| SCP1MTH | S. cerevisiae MAT-a/alpha ura3/ura3 leu2/leu2 his3/his3 trp1/trp1 ho/ho |
| | pdc1Δ::[P_{ScPDC1}:25ScCOX4-MTS:BsalsS:P_{ScTDH3}:LlkivD:ScHMI1- |
| | MTS:P_{ScADH1}:DmADH:P_{ScFBA1}:ScURA3:P_{ScFBA1}:ScILV5:P_{ScTPI1}:ScILV3:T_{ScPDC1}]/PDC1 |
| | pdc6Δ::[P_{ScTDH3}:Nc Transhydrogenase:P_{ScTEF1}:hph:]/PDC6 |
| SCP1N | S. cerevisiae MAT-a/alpha ura3/ura3 leu2/leu2 his3/his3 trp1/trp1 ho/ho |
| | pdc1Δ::[P_{PDC1}:25COX4-MTS:BsalsS:P_{TDH3}:LlkivD:ScHMI1- |
| | MTS:P_{ScADH1}:DmADH:P_{ScFBA1}:ScURA3:P_{ScFBA1}:31COX4-MTS:EcilvCcoSc^{P2D1-A1-his6}: |
| | P_{ScTPI1}:ILV3:T_{ScPDC1}]/PDC1 |
| SCP1TH | S. cerevisiae MAT-a/alpha ura3/ura3 leu2/leu2 his3/his3 trp1/trp1 ho/ho |
| | pdc1Δ::[P_{ScPDC1}:25ScCOX4-MTS:BsalsS:P_{ScTDH3}:LlkivD:ScHMI1- |
| | MTS:P_{ScADH1}:ScADH7:P_{ScFBA1}:ScURA3:P_{ScFBA1}:ScILV5:P_{ScTPI1}:ScILV3:T_{ScPDC1}]/PDC1 |
| | pdc6 Δ::[P_{ScTDH3}:Nc Transhydrogenase:P_{ScTEF1}:hph]/PDC6 |
| SCPA1 | S. cerevisiae MAT-a/alpha ura3/ura3 leu2/leu2 his3/his3 trp1/trp1 ho/ho |
| | pdc1Δ::[P_{ScPDC1}:25ScCOX4-MTS:BsalsS:P_{ScTDH3}:LlkivD:ScHMI1- |
| | MTS:P_{ScADH1}:ScADH3- |
| | MTS:ScADH7:P_{ScFBA1}:ScURA3:P_{ScFBA1}:ScILV5:P_{ScTPI1}:ScILV3:T_{ScPDC1}]/PDC1 |
| SCPA1MB | S. cerevisiae MAT-a/alpha ura3/ura3 leu2/leu2 his3/his3 trp1/trp1 ho/ho |
| | pdc1Δ::[P_{ScPDC1}:25ScCOX4-MTS:BsalsS:P_{ScTDH3}:LlkivD:ScHMI1- |
| | MTS:P_{ScADH}:ScADH3- |
| | MTS:ScADH7:P_{ScFBA1}:ScURA3:P_{ScFBA1}:ScILV5:P_{ScTPI1}:ScILV3:T_{ScPDC1}]/PDC1 |
| | pdc6 Δ::[P_{ScTDH3}:ScPCK1:P_{ScADH1}:ScMDH2:P_{ScFBA1}:ScDIC1:P_{ScTPI1}:ScMAE1: |
| | P_{ScTEF1}:hph:T_{ScPDC6}]/PDC6 |
| SCPA1N | S. cerevisiae MAT-a/alpha ura3/ura3 leu2/leu2 his3/his3 trp1/trp1 ho/ho |
| | pdc1Δ::[P_{PDC1}:25COX4-MTS:BsalsS:P_{TDH3}:LlkivD:ScHMI1-MTS: |
| | P_{ScADH1}:ScADH3-MTS:DmADH:P_{ScFBA1}:URA3: P_{ScFBA1}:31COX4- |
| | MTS:EcilvCcoSc^{P2D1-A1-his6}:P_{ScTPI1}:ILV3:T_{ScPDC1}]/PDC1 |
| SCPA1TH | S. cerevisiae MAT-a/alpha ura3/ura3 leu2/leu2 his3/his3 trp1/trp1 ho/ho |
| | pdc1Δ::[P_{ScPDC1}:25ScCOX4-MTS:BsalsS:P_{ScTDH3}:LlkivD:ScHMI1- |
| | MTS:P_{ScADH}:ScADH3- |
| | MTS:ScADH7:P_{ScFBA1}:ScURA3:P_{ScFBA1}:ScILV5:P_{ScTPI1}:ScILV3:T_{ScPDC1}]/PDC1 |
| | pdc6 Δ::[P_{ScTDH3}:Nc Transhydrogenase:P_{ScTEF1}:hph]/PDC6 |

| pGV No. | SEQ ID NO | Genotype |
|---|---|---|
| 1104 | | ScTRP1 2μ |
| 1315 | | P_{ScTEF2}:ScILV2ΔN:P_{ScTDH3}:ScILV6ΔN:T_{ScCYC1}, bla, pUC ori, HIS3, |
| | | 2μ |
| 1441 | | P_{ScTEF1}:MCS:T_{ScCYC1} ScHIS3 2μ pUC ori bla |
| 1503 | | P_{ScTEF1}:G418, bla, pUC ori, |
| 1590 | | P_{ScTEF1}:LlkivD2:P_{Sc}_{_{—}}_{TDH3}:ScADH7:URA3, PMB1, AP^{r} |
| 1730 | | P_{ScPDC}, ScTRP1, P_{ScCUP1-1}:BsalsS2::T_{Sc}_{_{—}}_{PDC1}, PMB1, AP^{r} |
| 1731 | | P_{ScPDC5},ScLEU, P_{ScTEF1}:EcilvDΔNcoKl:P_{ScTDH3}:EcilvCΔN: T_{ScPDC5} |
| | | PMB1, AP^{r} |
| 1733 | | P_{ScPDC6}, ScURA3 P_{ScTEF1}:LlkivD2:P_{ScTDH3}:ScADH7:T_{ScPDC6} PMB1, |
| | | AP^{r} |
| 1773 | 1 | P_{Sc}_{_{—}}_{PDC1}:BsalsS:P_{ScTDH3}:LlkivD:P_{ScADH1}:ScADH7:P_{ScFBA1}:5′ ScURA3, |
| | | pUC ori, kan^{R} |
| 1774 | 2 | 3′ ScURA3:P_{S}_{_{—}}_{FBA1}:EcilvCco:P_{ScTPI1}:EcilvDco:T_{ScPDC1}, pUC ori, |
| | | kan^{R} |
| 1799 | | KmURA3D, pUC ori, AP^{r} |
| 1810 | 3 | 3′ URA3:P_{ScFBA1}:ScILV5:P_{ScTPI1}:EcilvDco:T_{ScPDC1}, pUC ori, kan^{R} |
| 1811 | 4 | P_{Km}_{_{—}}_{PDC1}: BsalsS:P_{ScTDH3}:LlkivD:P_{ScADH1}:ScADH7:P_{ScFBA1}:5′ |
| | | ScURA3, pUC ori, kan^{R} |
| 1812 | 5 | 3′ ScURA3:P_{ScFBA}:EcilvCco:P_{ScTPI1}:EcilvDco:T_{KmPDC1}, pUC ori, |
| | | kan^{R} |
| 1816 | | P_{Sc}_{_{—}}_{PDC1}: BsalsS:T_{ScTDH3}:Llkivd::P_{ScADH1}:ScADH7:P_{ScFBA1}:URA3: |
| | | T_{ScPDC1}, pUC ori, kan^{R} |
| 1817 | 6 | 3′ ScURA3:P_{ScFB1}:ScILV5:P_{ScTPI1}:ScILV3:T_{ScPDC1}, pUC ori, kan^{R} |
| 1817N | 12 | 3′ ScURA3: P_{ScFBA1}:31COX4-MTS:EcilvCcoSc^{P2D1-A1-his6}: P_{ScTPI1}: |
| | | ScILV3: T_{ScPDC1}, pUC ori, kan^{R} |
| 1832 | 7 | 3′ ScURA3:P_{ScFBA}:ScILV5:P_{ScTPI1}:ScILV3:T_{KmPDC1}, pUC ori, kan^{R} |
| 1832N | 13 | 3′ ScURA3: P_{ScFBA1}:31COX4-MTS:EcilvCcoSc^{P2D1-A1-his6}: P_{ScTPI1}: |
| | | ScILV3: T_{KmPDC1}, pUC ori, kan^{R} |
| 1834 | 8 | P_{KmPDC1}:25ScCOX4-MTS:BsalsS:P_{ScTDH3}:LlkivD:ScHMI1- |
| | | MTS:P_{ScADH1}:ScADH7:P_{ScFBA}:5′ ScURA3, pUC ori, kan^{R} |
| 1834N | 14 | P_{KmPDC1}:25ScCOX4-MTS:BsalsS:P_{ScTDH3}:LlkivD:ScHMI1- |
| | | MTS:P_{ScADH1}:DmADH:P_{ScFBA1}:5′ ScURA3, pUC ori, kan^{R} |
| 1874 | | P_{ScPDC1}:25ScCOX4-MTS:BsalsS:P_{ScTDH3}:LlkivD:ScHMI1- |
| | | MTS:P_{ScADH1}:ScADH7:P_{ScFBA1}:ScURA3:T_{ScPDC1}, pUC ori, kan^{R} |
| 1875 | 92 | P_{ScPDC1}:31ScCOX4-MTS:BsalsS:P_{ScTDH3}:LlkivD:ScHMI1-MTS:P_{ScADH1}: |
| | | ScADH7: P_{ScFBA1}:ScURA3:T_{ScPDC1}, pUC ori, kan^{R} |
| 1876 | 97 | P_{ScPDC1}:25ScCOX4-MTS:BsalsS:P_{ScTDH3}ScARO10:ScHMI1-MTS: |
| | | P_{ScADH1}:ScADH7:P_{ScFBA1}:ScURA3:T_{ScPDC1}, pUC ori, kan^{R} |
| 1877 | | P_{ScPDC1}:31ScCOX4-MTS:BsalsS:P_{ScTDH3}:ScARO10:ScHMI1-MTS: |
| | | P_{ScADH1}:ScADH7: P_{ScFBA1}:ScURA3:T_{ScPDC1}, pUC ori, kan^{R} |
| 1878 | | P_{ScPDC1}:ScILV2:P_{ScTDH3}:Llkivd:ScHMI1-MTS:P_{ScADH1}:ScADH7: |
| | | P_{ScFBA1}:ScURA3:T_{ScPDC1}, pUC ori, kan^{R} |
| 1879 | | P_{ScPDC1}:ScILV2:P_{ScTDH3}:ScARO10::ScHMI1- |
| | | MTS:P_{ScADH1}:ScADH7:P_{ScFBA1}:ScURA3:T_{ScPDC}, pUC ori, kan^{R} |
| 1892 | | P_{ScPDC1}:ScILV2:P_{ScTDH3}:Llkivd:ScHMI1-MTS:P_{ScADH1}:P_{ScFBA1}: |
| | | ScURA3:T_{S}_{_{—}}_{PDC, pUC ori, kan}^{R} |
| 1909 | | P_{ScPDC1}:31ScCOX4-MTS:BsalsS:P_{ScTDH3}:LlkivD:ScHMI1- |
| | | MTS:P_{ScFBA1}:ScURA3:T_{ScPDC} pUC ori Kan^{R} |
| 1914 | | P_{TEF1}:Llkivd2:P_{TDH3}:DmADH PDC6 5′,3′ targeting homology |
| | | URA3 |
| 1936 | | P_{TEF1}:ScILV3ΔN P_{TDH3}:EcilvCco(Sc)^{Q110V} PDC5 5′,3′ targeting |
| | | homology LEU2 |
| 1990 | 94 | P_{Sc}_{_{—}}_{PDC1}:31ScCOX4- |
| | | MTS:BsalsS:P_{ScTDH3}:LlkivD2:P_{ScFBA1}:ScURA3:T_{ScPDC} pUC ori |
| | | Kan^{R} |
| 1999 | 93 | P_{ScTEF1}:ScBAT2:T_{ScCYC1} ScHIS3 2μ |
| 2015 | 95 | P_{Sc}_{_{—}}_{PDC1}:31ScCOX4-MTS:BsalsS:P_{Sc}_{_{—}}_{TDH3}:P_{Sc}_{_{—}}_{FBA1}:ScURA3: |
| | | T_{Sc}_{_{—}}_{PDC} pUC ori, Kan^{R} |
| 2030 | | P_{ScTEF1}:LlkivD2:P_{ScTDH3}:DmADH, pUC ori, kan^{R} |
| 2061 | | P_{KmPDC1} LlkivD2:P_{ScTDH3}DmADH:P_{Sc FBA1}ScURA3 (5′ fragment), |
| | | pUC ori, kan^{R} |
| 2062 | | 3′ fragment of ScURA3: P_{ScFBA1}31ScCOX4-MTS:BsalsS:T_{KmPDC1}, |
| | | pUC ori, kan^{R} |
| 2212 | 98 | P_{TDH3}:ScBAT1:T_{ScCYC1} ScTRP1 2μ |
| 7001 | 9 | P_{ScPDC1}:25ScCOX4-MTS:BsalsS:P_{ScTDH3}:LlkivD:ScHMI1- |
| | | MTS:P_{ScADH1}:ScADH7:P_{ScFBA1}:ScURA3 (5′ fragment), pUC ori, |
| | | kan^{R} |
| 7001N | 15 | P_{Km}_{_{—}}_{PDC1}:25ScCOX4-MTS:BsalsS:P_{ScTDH3}:LlkivD:ScHMI1- |
| | | MTS:P_{ScADH1}:DmADH:P_{ScFBA1}:ScURA3 (5′ fragment), pUC ori, |
| | | kan^{R} |
| 7101 | 10 | P_{Sc}_{_{—}}_{PDC1}:25ScCOX4-MTS:BsalsS:P_{ScTDH3}:LlkivD:ScHMI1- |
| | | MTS-P_{ScADH1}:ScADH3-MTS:ScADH7:P_{ScFBA}:Sc_URA3(5′ |
| | | fragment), pUC ori, kan^{R} |
| 7101N | 16 | P_{Sc}_{_{—}}_{PDC1}:25ScCOX4-MTS:BsalsS:P_{ScTDH3}:LlkivD:ScHMI1- |
| | | MTS:P_{ScADH1}:ScADH3-MTS:DmADH:P_{ScFBA}:ScURA3 (5′ |
| | | fragment), pUC ori, kan^{R} |
| 8000 | 18 | P_{ScTDH3}:ScPCK1:P_{ScADH1}:ScMDH2:P_{ScFBA1}:ScDIC1:P_{ScTPI1}: |
| | | ScMAE1:P_{ScTEF1}:hph:T_{ScPDC6}, pUC ori, kan^{R} |
| 9000 | 19 | P_{ScTDH3}:Nc Transhydrogenase:P_{ScTEF1}:hph:T_{ScPDC6}:P_{ScPDC6}, pUC ori, |
| | | kan^{R} |
| 9834 | 11 | P_{KmPDC1}:25ScCOX4-MTS:BsalsS:P_{ScTDH3}:LlkivD:ScHMI1- |
| | | MTS:P_{ScADH1}:ScADH3-MTS:ScADH7:P_{ScFBA1}:ScURA3 (5′ |
| | | fragment) pUC ori, kan^{R} |
| 9834N | 17 | P_{KmPDC1}:25ScCOX4-MTS:BsalsS:P_{ScTDH3}:LlkivD:ScHMI1- |
| | | MTS:P_{ScADH1}:ScADH3-MTS:DmADH:P_{ScFBA1}:ScURA3 (5′ |
| | | fragment), pUC ori, kan^{R} |

Table 3 outlines the primers sequences disclosed herein:

| GEVO No. | Sequence |
|---|---|
| 393 | ATGGAAGATGTTCACCGTGC (SEQ ID NO: 100) |
| 394 | CAACCACTGCAGCCAGCAGTTAAAGCACCAAC (SEQ ID NO: 163) |
| 395 | CTCACAGGATCCGACGCAATGCCATTACACTC (SEQ ID NO: 101) |
| 457 | CAGATCGTCGACATGACCTTGGCACCCCTAGAC (SEQ ID NO: 99) |
| 458 | GTTCCCGGATCCTCAGTTCAAATCAGTAACAACCCTTG (SEQ ID NO: 55) |
| 562 | TTAAGCGGATCTGCCTACTC (SEQ ID NO: 164) |
| 837 | ATCATTTGTAAACTGGAACAGC (SEQ ID NO: 165) |
| 1321 | AATCATATCGAACACGATGC (SEQ ID NO: 102) |
| 1322 | TCAGAAAGGATCTTCTGCTC (SEQ ID NO: 103) |
| 1323 | ATCGATATCGTGAAATACGC (SEQ ID NO: 104) |
| 1324 | AGCTGGTCTGGTGATTCTAC (SEQ ID NO: 105) |
| 1341 | TGCTGAAAGAGAAATTGTCC (SEQ ID NO: 106) |
| 1342 | TTTCTTGTTCGAAGTCCAAG (SEQ ID NO: 107) |
| 1364 | TTTTGCGGCCGCTTAGATGCCGGAGTCCCAGTGCTTG |
| | (SEQ ID NO: 54) |
| 1435 | GTTGGCATAGCGGAAACTTC (SEQ ID NO: 65) |
| 1436 | AAATGACGACGAGCCTGAAG (SEQ ID NO: 108) |
| 1437 | GACCTGACCATTTGATGGAG (SEQ ID NO: 109) |
| 1439 | CAATTGGCGAAGCAGAACAAG (SEQ ID NO: 110) |
| 1440 | ATCGTACATCTTCCAAGCATC (SEQ ID NO: 111) |
| 1441 | AATCGGAACCCTAAAGGGAG (SEQ ID NO: 112) |
| 1442 | AATGGGCAAGCTGTTTGCTG (SEQ ID NO: 113) |
| 1443 | TGCAGATGCAGATGTGAGAC (SEQ ID NO: 114) |
| 1566 | TCCCACCCAATCAAGGCCAACG (SEQ ID NO: 115) |
| 1567 | TCCACCTGGTGCCAATGAACCG (SEQ ID NO: 116) |
| 1587 | CGGCTGCCAGAACTCTACTAACTG (SEQ ID NO: 117) |
| 1588 | GCGACGTCTACTGGCAGGTTAAT (SEQ ID NO: 118) |
| 1595 | CAACCTGGTGATTTGGGGAAG (SEQ ID NO: 119) |
| 1597 | GAATGATGGCAGATTGGGCA (SEQ ID NO: 120) |
| 1598 | TATTGTGGGGCTGTCTCGAATG (SEQ ID NO: 121) |
| 1608 | GTCAACATGTCGACTGCAATTATTTGGTTTGGGT |
| | (SEQ ID NO: 56) |
| 1609 | GTGGTTAGGGATCCAATCGATCCAAAGAAGAGAG |
| | (SEQ ID NO: 57) |
| 1610 | TGCAGAGTCGAATTCAAGCTTGTGTATATGCCAAA |
| | (SEQ ID NO: 58) |
| 1615 | CAACTCGCGGCCGCGGATCCTAGGTTATTGGTTTTCTGGTCTCAAC |
| | (SEQ ID NO: 59) |
| 1616 | CGCCGACTCGAGATGTTGAGAACTCAAGCCGC (SEQ ID NO: 60) |
| 1617 | CGTTGAGTCGACATGGGCTTGTTAACGAAAGTTGC |
| | (SEQ ID NO: 61) |
| 1618 | GCCAACGGATCCTCAAGCATCTAAAACACAACCG |
| | (SEQ ID NO: 62) |
| 1633 | TCCGTCACTGGATTCAATGCCATC (SEQ ID NO: 122) |
| 1634 | TTCGCCAGGGAGCTGGTGAA (SEQ ID NO: 123) |
| 1652 | ATTTCTTTCCAGACTTGTTC (SEQ ID NO: 63) |
| 1653 | ATCTCCCCACTTCAGAAGTTCCTA (SEQ ID NO: 64) |
| 1658 | TAGGAACTTCTGAAGTGGGGAGATCTCACCAGTAAAACATACGCATACA |
| | CATAC (SEQ ID NO: 66) |
| 1661 | ATCCAAAATTTTTACGTAACTGATTGTATCGCTGCACATTATAACCATGT |
| | GTCACGGATTTGTTTTGTTCGGCG (SEQ ID NO: 69) |
| 1662 | ATCCAAAATTTTTACGTAACTGATTGTATCGCTGCACATTATAACCATGT |
| | GTCACTTTTTTATTTCTTTTAAGTGCCGC (SEQ ID NO: 70) |
| 1663 | AATCAGTTACGTAAAAATTTTGGATTTTATAGAGCATATTCTTCACTAAG |
| | AGGTTGTAAGAGCGTTTTCAGACGTATATAGGCTAGCTAAT |
| | (SEQ ID NO: 71) |
| 1664 | ATTAGCTAGCCTATATACGTCTGAAAACGCTCTTACAACCTCTTAGTGAA |
| | GAATATGCTCTATAAAATCCAAAATTTTTACGTAACTGATT |
| | (SEQ ID NO: 72) |
| 1665 | TACTCGAGATGCTTTCACTACGTCAATCTATAAGATTTTTCAAGCCAGCC |
| | ACAAGAACTTTGTGTAGCTCTAGATATCTGCTT (SEQ ID NO: 73) |
| 1666 | AAGCAGATATCTAGAGCTACACAAAGTTCTTGTGGCTGGCTTGAAAAATC |
| | TTATAGATTGACGTAGTGAAAGCATCTCGAGTA (SEQ ID NO: 74) |
| 1667 | ACTCGAGATGCTTTCACTACGTCAATCTATAAGATTTTTCAAGCCAGCCA |
| | CAAGAACTTTGTGTAGCTCTAGATATCTGCTTCAGCAAAAACCCGTGGTG |
| | (SEQ ID NO: 75) |
| 1668 | CACCACGGGTTTTTGCTGAAGCAGATATCTAGAGCTACACAAAGTTCTTG |
| | TGGCTGGCTTGAAAAATCTTATAGATTGACGTAGTGAAAGCATCTCGAGT |
| | (SEQ ID NO: 76) |
| 1671 | CACATAGAGCAAGCAAGCAG (SEQ ID NO: 124) |
| 1672 | CGTAAGCAGCGTTCAATTCG (SEQ ID NO: 125) |
| 1673 | CGAATTGAACGCTGCTTACGGTGAATTCGAGCTCATAGCTTC |
| | (SEQ ID NO: 126) |
| 1674 | CATTTGGACACCTGGGAAAGGCTTACGCAATGCCATTACAC |
| | (SEQ ID NO: 127) |
| 1675 | CTTTCCCAGGTGTCCAAATG (SEQ ID NO: 128) |
| 1676 | GAGCTTGCTTGACCAAGTTG (SEQ ID NO: 129) |
| 1678 | CTGGCATTGTGTCTGGATTG (SEQ ID NO: 130) |
| 1679 | GAGATTAAATCGCGCTAGCTTAATTCTGCTGACCACATCTTC |
| | (SEQ ID NO: 131) |
| 1680 | CAGAATTAAGCTAGCGCGATTTAATCTCTAATTATTAGT |
| | (SEQ ID NO: 132) |
| 1681 | TTTGTGTAGCTCTAGATATCTGCTTATGTTGACTAAAGCTACAAAAGAGC |
| | (SEQ ID NO: 77) |
| 1682 | TCTGCTTCAGCAAAAACCCGTGGTGATGTTGACTAAAGCTACAAAAGAGC |
| | (SEQ ID NO: 78) |
| 1683 | TACTCGAGATGCTTTCACTACG (SEQ ID NO: 79) |
| 1684 | ATTAGCTAGCCTATATACGTCTGA (SEQ ID NO: 80) |
| 1685 | ATTACTCGAGATGATCAGACAATCTACGCTAA (SEQ ID NO: 81) |
| 1686 | CTGAAAAAGCGTGTTTTTTATGGATCCTCAGTGCTTACCGCCTGTAC |
| | (SEQ ID NO: 133) |
| 1687 | GTACAGGCGGTAAGCACTGAGGATCCATAAAAAACACGCTTTTTCAG |
| | (SEQ ID NO: 134) |
| 1688 | TTCAATTGTAACAGGTGCCATAAGCTTTTTGTTTGTTTATGTGTG |
| | (SEQ ID NO: 135) |
| 1689 | CACACATAAACAAACAAAAAGCTTATGGCACCTGTTACAATTGAA |
| | (SEQ ID NO: 136) |
| 1690 | ATAAAAAACACGCTTTTTCAG (SEQ ID NO: 137) |
| 1691 | TTTGTTTGTTTATGTGTGTTTATTC (SEQ ID NO: 138) |
| 1738 | GCCTTCTCGTCAACCAAGA (SEQ ID NO: 139) |
| 1739 | CGTGAATGTAAGCGTGACATAAC (SEQ ID NO: 140) |
| 1740 | AAATCATATGTGCTACCATGGTGCGTTG (SEQ ID NO: 141) |
| 1741 | TCTTGGTTGACGAGAAGGCG (SEQ ID NO: 142) |
| 2038 | CACATAAACAAACAAAAAGCTTATGTATACTGTTGGTGATTA |
| | (SEQ ID NO: 143) |
| 2039 | CAGTATTGTTATGCGGCCGCTTAGGATTTATTCTGTTCAG |
| | (SEQ ID NO: 144) |
| 2105 | ACTAGACTCGAGATGCTTTCACTACGTCAATCTA (SEQ ID NO: 145) |
| 2106 | ATCTGAGGATCCTTATAAGGCTTTGGTCTTCAT (SEQ ID NO: 146) |
| 2305 | TTGATTGGATCCATGTTGCAGAGACATTCCT (SEQ ID NO: 147) |
| 2323 | ATTGATGCGGCCGCTTAGTTCAAGTCGGCAACA (SEQ ID NO: 148) |
| ADH3MTSF | AATTCATATGTTGAGAACGTCAACATTGTTCACCAGGCGTGTCCAACCAA |
| | GCCTATTTTCTAGAAACATTCTTAGATTGCAATCCACAGCTGCATTATA |
| | (SEQ ID NO: 82) |
| ADH3MTSR | TATAATGCAGCTGTGGATTGCAATCTAAGAATGTTTCTAGAAAATAGGCT |
| | TGGTTGGACACGCCTGGTGAACAATGTTGACGTTCTCAACATATGAATT |
| | (SEQ ID NO: 83) |
| ADH7F | ATCCACAGCTGCATTATATCCAGAGAAATTCCAAGGC (SEQ ID NO: 84) |
| ADH7R | GCTCCCATGGCCTTAGCTAG (SEQ ID NO: 85) |
| ADH37F | AATTCATATGTTGAGAACGTCAACA (SEQ ID NO: 86) |
| DMADH3MTSF | AATTCATATGTTGAGAACGTCAACATTGTTCACCAGGCGTGTCCAACCAA |
| | GCCTATTTTCTAGAAACATTCTTAGATTGCAATCCACAGCTGCATCGTT |
| | (SEQ ID NO: 87) |
| DMADH3MTSR | AACGATGCAGCTGTGGATTGCAATCTAAGAATGTTTCTAGAAAATAGGCT |
| | TGGTTGGACACGCCTGGTGAACAATGTTGACGTTCTCAACATATGAATT |
| | (SEQ ID NO: 88) |
| DMADH3R | AATTGCGGCCGCTTAGATGCCGGAGTCCCAG (SEQ ID NO: 89) |
| DMADH3F | ATCCACAGCTGCATCGTTTACTTTGACCAACAAG (SEQ ID NO: 90) |
| DMADHF | TTTTCATATGTCGTTTACTTTGACCAACAAG (SEQ ID NO: 91) |

### Example 1

### Mitochondrial Production of Isobutanol in K. marxianus with ALS, KARI, DHAD, and KIVD Targeted to the Mitochondria, Wherein PDC is Deleted

This example illustrates how mitochondrial isobutanol production is achieved in PDC-minus yeast using four pathway enzymes targeted to the mitochondria. With these modifications, strain A will generally exhibit higher isobutanol productivity, titer, and/or yield as compared to the parent strain GEVO1947, while at the same time, exhibiting reduced ethanol production.

To accomplish this, GEVO1947 is first transformed with linear DNA from pGV1832 and additional linear DNA from pGV1834 resulting in strain A. This combination of linear DNA contains genes coding for all five isobutanol pathway enzymes, the K. marxianus PDC1 promoter (P_{KmPDC1}) and terminator (T_{KmPDC1}) sequences for homologous integration into the PDC1 locus and the uracil marker for selection. This homologous replacement event results in the simultaneous integration of the isobutanol pathway along with deletion of the PDC1 coding sequence. Adh7p is targeted to its native compartment, the cytosol. All other pathway enzymes are targeted to the mitochondria. Pathway enzymes that are not natively localized to the mitochondria are fused to mitochondrial targeting sequences that direct them to the mitochondria. The transformed cells are plated onto selective medium without uracil and incubated at 30° C. for 3 days. After 3 days colonies are patched onto selective plates and these patches are used for colony PCR and RT PCR to verify correct integration and transcription of the pathway genes. Three verified clones from each transformation are used to inoculate 3 mL overnight cultures in YPD. As a control GEVO1947 is used. The overnight cultures are used to inoculate 100 mL YPD cultures in 1 L shake flasks. These cultures are harvested at an OD600 of 0.6-0.8. The cells are resuspended in 50 mL fresh YPD medium and the cultures are incubated in 250 mL shake flasks at 30° C., 250 rpm. 2 mL samples are taken at 24 and 48 hours after inoculation. The fermentation is ended after 48 hours. Samples are processed and analyzed by HPLC and GC.

### Example 2

### Mitochondrial Production of Isobutanol in K. Marxianus with all Pathway Enzymes Targeted to the Mitochondria, Wherein PDC is Deleted

This example illustrates how mitochondrial production of isobutanol is achieved in PDC-minus yeast with five pathway enzymes targeted to the mitochondria. With these modifications, strain A1 will generally exhibit higher isobutanol productivity, titer, and/or yield as compared to the parent strain GEVO1947, while at the same time, exhibiting reduced ethanol production.

To accomplish this, GEVO1947 first is transformed with linear DNA from pGV1832 and additional linear DNA from pGV9834 resulting in strain A1. The combination of linear DNA contains genes coding for all five isobutanol pathway enzymes, the K. marxianus PDC1 promoter and terminator sequences for homologous integration into the PDC1 locus and the uracil marker for selection. This homologous replacement event results in the simultaneous integration of the isobutanol pathway along with the deletion of the PDC1 coding sequence. All pathway enzymes are targeted to the mitochondria. Pathway enzymes that are not natively localized to the mitochondria are fused to mitochondrial targeting sequences that direct them to the mitochondria. The transformed cells are plated onto selective medium without uracil and incubated at 30° C. for 3 days. After 3 days colonies are patched onto selective plates and these patches are used for colony PCR and RT PCR to verify correct integration and transcription of the pathway genes. Three verified clones from each transformation are used to inoculate 3 mL overnight cultures in YPD. As a control GEVO1947 is used. These cultures are used to inoculate 100 mL YPD cultures in 1 L shake flasks. These cultures are harvested at an OD600 of 0.6-0.8. The cells are resuspended in 50 mL fresh YPD medium and the cultures are incubated in 250 mL shake flasks at 30° C., 250 rpm. 2 mL samples are taken at 24 and 48 hours after inoculation. The fermentation is ended after 48 hours. Samples are processed and analyzed by HPLC and GC.

### Example 3

### Mitochondrial Production of Isobutanol in K. Marxianus with all Pathway Enzymes Targeted to the Mitochondria

This example illustrates how mitochondrial production of isobutanol is achieved in yeast with five pathway enzymes targeted to the mitochondria. With these modifications, strain A1P will generally exhibit higher isobutanol productivity, titer, and/or yield as compared to the parent strain GEVO1947.

To accomplish this, GEVO1947 is first transformed with linear DNA from pGV1817 and additional linear DNA from pGV7101 resulting in strain A1P. This combination of linear DNA contains genes coding for all five isobutanol pathway enzymes, and the uracil marker for selection. The transformation leads to random (non targeted) insertion of the isobutanol pathway into the K. marxianus genome. All pathway enzymes are targeted to the mitochondria. Pathway enzymes that are not natively localized to the mitochondria are fused to mitochondrial targeting sequences that direct them to the mitochondria. The transformed cells are plated onto selective medium without uracil and incubated at 30° C. for 3 days. After 3 days colonies are patched onto selective plates and these patches are used for colony PCR and RT-PCR to verify correct integration and transcription of the pathway genes. Three verified clones from each transformation are used to inoculate 3 mL overnight cultures in YPD. As a control GEVO1947 is used. These cultures are used to inoculate 100 mL YPD cultures in 1 L shake flasks. These cultures are harvested at an OD600 of 0.6-0.8. The cells are resuspended in 50 mL fresh YPD medium and the cultures are incubated in 250 mL shake flasks at 30° C., 250 rpm. 2 mL samples are taken at 24 and 48 hours after inoculation. The fermentation is ended after 48 hours. Samples are processed and analyzed by HPLC and GC.

### Example 4

### Mitochondrial Production of Isobutanol in S. cerevisiae with all Pathway Enzymes Targeted to the Mitochondria

This example illustrates how mitochondrial production of isobutanol is achieved in yeast with five pathway enzymes targeted to the mitochondria. With these modifications, strain SCPA1 will generally exhibit higher isobutanol productivity, titer, and/or yield as compared to the parent strain GEVO1186.

To accomplish this, GEVO1186, a diploid CEN.PK strain, is first transformed with linear DNA from pGV1817 and additional linear DNA from either pGV7101 resulting in strain SCPA1. Each combination of linear DNA contains genes coding for all five isobutanol pathway enzymes, the S. cerevisiae PDC1 promoter and terminator sequences for homologous integration into the PDC1 locus and the uracil marker for selection. This homologous replacement event results in the simultaneous integration of the isobutanol pathway along with the deletion of the PDC1 coding sequence. All pathway enzymes are targeted to the mitochondria. Isobutanol pathway enzymes that are not natively localized to the mitochondria are fused to mitochondrial targeting sequences that direct them to the mitochondria. The transformed cells are plated onto selective medium without uracil and incubated at 30° C. for 3-4 days. After 3-4 days colonies are patched onto selective plates and these patches are used for colony PCR and RT-PCR to verify correct integration and transcription of the pathway genes. Three verified clones from each transformation are used to inoculate 3 mL overnight cultures in YPD. As a control GEVO1186 is used. These overnight cultures are used to inoculate 100 mL YPD cultures in 1 L shake flasks. The cultures are harvested at an OD600 of 0.6-0.8. The cells are resuspended in 50 mL fresh YPD medium and the cultures are incubated in 250 mL shake flasks at 30° C., 250 rpm. 2 mL samples are taken at 24 and 48 hours after inoculation. The fermentation is ended after 48 hours. Samples are processed and analyzed by HPLC and GC.

### Example 5

### Mitochondrial Production of Isobutanol in a PDC-Minus S. Cerevisiae with all Pathway Enzymes Targeted to the Mitochondria

This example illustrates how mitochondrial production of isobutanol is achieved in PDC-minus yeast with five pathway enzymes targeted to the mitochondria. With these modifications, strain SCA1 will generally exhibit higher isobutanol productivity, titer, and/or yield as compared to the parent strain GEVO7777, while at the same time, exhibiting reduced ethanol production.

To accomplish this, GEVO7777, a haploid CEN.PK strain deleted for PDC5 and PDC6, is first transformed with linear DNA from pGV1817 and additional linear DNA from pGV7101 resulting in strain SCA1. The combination of linear DNA contains genes coding for all five isobutanol pathway enzymes, the S. cerevisiae PDC1 promoter and terminator sequences for homologous integration into the PDC1 locus and the uracil marker for selection. This homologous replacement event results in the simultaneous integration of the isobutanol pathway along with the deletion of the PDC1 coding sequence. All pathway enzymes are targeted to the mitochondria. Pathway enzymes that are not natively localized to the mitochondria are fused to mitochondrial targeting sequences that direct them to the mitochondria. The transformed cells are plated onto selective medium using ethanol as carbon source and lacking uracil and incubated at 30° C. for 3-4 days. After 3-4 days colonies are patched onto selective plates and these patches are used for colony PCR and RT-PCR to verify correct integration and transcription of the pathway genes. Three verified clones from each transformation are used to inoculate 3 mL overnight cultures in YPEthanol. As a control GEVO7777 is used. These overnight cultures are used to inoculate 100 mL YPEthanol cultures in 1 L shake flasks. The cultures are harvested at an OD600 of 0.6-0.8. The cells are resuspended in 50 mL fresh YPD medium and the cultures are incubated in 250 mL shake flasks at 30° C., 250 rpm. 2 mL samples are taken at 24 and 48 hours after inoculation. The fermentation is ended after 48 hours. Samples are processed and analyzed by HPLC and GC.

### Example 6

### Mitochondrial Production of Isobutanol in C2-Independent PDC-Minus S. cerevisiae with ALS, KARI, DHAD, and KIVD Targeted to the Mitochondria

This example illustrates how mitochondrial production of isobutanol is achieved in C2-independent PDC-minus yeast with four pathway enzymes targeted to the mitochondria. With these modifications, strain c2i-SC1 will generally exhibit higher isobutanol productivity, titer, and/or yield as compared to the parent strain GEVO1863.

S. cerevisiae which is deleted for all PDC genes (PDC1, PDC5 and PDC6) is dependent on C2-carbons such as ethanol or acetate for growth. Such a strain can be evolved to grow on glucose and hence be C2-independent (van Maris, A., et al., Applied and Environmental Microbiology, 2004, 70(1):159-166). GEVO1863 is a PDC minus strain that has been evolved to grow on glucose. GEVO1863 is transformed with linear DNA from pGV1817 and additional linear DNA from pGV7001 resulting in strain c2i-SC1. The combination of linear DNA contains genes coding for all five isobutanol pathway enzymes, the S. cerevisiae PDC1 promoter and terminator sequences for homologous integration into the PDC1 locus and the uracil marker for selection. The homologous replacement event replaces the pdc1::ble allele while simultaneously integrating the isobutanol pathway. Adh7p is targeted to its native compartment, the cytosol. All other pathway enzymes are targeted to the mitochondria. Pathway enzymes that are not natively localized to the mitochondria are fused to mitochondrial targeting sequences that direct them to the mitochondria. The transformed cells are plated onto selective medium and lacking uracil and incubated at 30° C. for 3-4 days. After 3-4 days colonies are patched onto selective plates and these patches are used for colony PCR and RT PCR to verify correct integration and transcription of the pathway genes. Three verified clones from each transformation are used to inoculate 3 mL overnight cultures in YPD. As a control GEVO1863 is used. These overnight cultures are used to inoculate 100 mL YPD cultures in 1 L shake flasks. The cultures are harvested at an OD600 of 0.6-0.8. The cells are resuspended in 50 mL fresh YPD medium and the cultures are incubated in 250 mL shake flasks at 30° C., 250 rpm. 2 mL samples are taken at 24 and 48 hours after inoculation. The fermentation is ended after 48 hours. Samples are processed and analyzed by HPLC and GC.

### Example 7

### Mitochondrial Production of Isobutanol in C2-Independent PDC-Minus S. Cerevisiae with all Pathway Enzymes Targeted to the Mitochondria

This example illustrates how mitochondrial production of isobutanol is achieved in C2-independent PDC-minus yeast with five pathway enzymes targeted to the mitochondria. With these modifications, strain c2i-SCA1 will generally exhibit higher isobutanol productivity, titer, and/or yield as compared to the parent strain GEVO1863.

S. cerevisiae which is deleted for all PDC genes (PDC1, PDC5 and PDC6) is dependent on C2-carbons such as ethanol or acetate for growth. Such a strain can be evolved to grow on glucose and hence be C2-independent (van Maris, A., et al., Applied and Environmental Microbiology, 2004, 70(1):159-166). GEVO1863 is a PDC minus strain that has been evolved for growth on glucose. GEVO1863 is transformed with linear DNA from pGV1817 and additional linear DNA from pGV7101 resulting in strain c2i-SCA1. Each combination of linear DNA contains genes coding for all five isobutanol pathway enzymes, the S. cerevisiae PDC1 promoter and terminator sequences for homologous integration into the PDC1 locus and the uracil marker for selection. This homologous replacement event simultaneous integrates the isobutanol pathway along with replacing the pdc1::ble allele. All pathway enzymes are targeted to the mitochondria. Pathway enzymes that are not natively localized to the mitochondria are fused to mitochondrial targeting sequences that direct them to the mitochondria. The transformed cells are plated onto selective medium lacking uracil and incubated at 30° C. for 3-4 days. After 3-4 days colonies are patched onto selective plates and these patches are used for colony PCR and RT-PCR to verify correct integration and transcription of the pathway genes. Three verified clones from each transformation are used to inoculate 3 mL overnight cultures in YPD. As a control GEVO1863 is used. These overnight cultures are used to inoculate 100 mL YPD cultures in 1 L shake flasks. The cultures are harvested at an OD600 of 0.6-0.8. The cells are resuspended in 50 mL fresh YPD medium and the cultures are incubated in 250 mL shake flasks at 30° C., 250 rpm. 2 mL samples are taken at 24 and 48 hours after inoculation. The fermentation is ended after 48 hours. Samples are processed and analyzed by HPLC and GC.

### Example 8

### Mitochondrial Production of Isobutanol in K. Marxianus with Cofactor Usage Balanced by NADH Dependent Kari and ADH, Wherein ALS, KARI, DHAD, and KIVD are Targeted to the Mitochondria

This example illustrates how mitochondrial production of isobutanol in yeast possessing NADH dependent KARI and ADH enzymes. With these modifications, strain AN will generally exhibit higher isobutanol productivity, titer, and/or yield as compared to strain A and the parent strain GEVO1947, while at the same time, exhibiting reduced ethanol production.

As disclosed herein, one approach to balance the cofactor usage between glycolysis and the isobutanol pathway is the use of NADH dependent KARI and ADH. To accomplish this, the mitochondrially targeted NADH dependent KARI is cloned into pGV1832 replacing ScILV5 to generate pGV1832N. The NADH dependent ADH DmADH is cloned into pGV1834 replacing ScADH7 to generate pGV1834N.

GEVO1947 is then transformed with linear DNA from pGV1832N and from pGV1834N resulting in strain AN. The combination of linear DNA contains genes coding for all five isobutanol pathway enzymes, the K. marxianus PDC1 promoter and terminator sequences for homologous integration into the PDC1 locus and the uracil marker for selection. This homologous replacement event results in the simultaneous integration of the isobutanol pathway along with the deletion of the PDC1 coding sequence. The NADH dependent ADH is not targeted to the mitochondria. All other pathway enzymes are targeted to the mitochondria. Pathway enzymes that are not natively localized to the mitochondria are fused to mitochondrial targeting sequences that direct them to the mitochondria. The transformed cells are plated onto selective medium without uracil and incubated at 30° C. for 3 days. After 3 days colonies are patched onto selective plates and these patches are used for colony PCR and RT-PCR to verify correct integration and transcription of the pathway genes. Three verified clones from each transformation are used to inoculate 3 mL overnight cultures in YPD. As controls, the parental strain GEVO1947 and strain A which contains the NADPH dependent isobutanol pathway, are used. These cultures are used to inoculate 100 mL cultures in 1 L shake flasks. These cultures are harvested at an OD600 of 0.6-0.8. The cells are resuspended in 50 mL fresh YPD medium and the cultures are incubated in 250 mL shake flasks at 30° C., 250 rpm. 2 mL samples are taken at 24 and 48 hours after inoculation. The fermentation is ended after 48 hours. Samples are processed and analyzed by HPLC and GC.

### Example 9

### Mitochondrial Production of Isobutanol in S. Cerevisiae with Cofactor Usage Balanced by NADH Dependent KARI and ADH, Wherein ALS, KARI, DHAD, and KIVD are Targeted to the Mitochondria

This example illustrates how mitochondrial production of isobutanol is achieved in yeast possessing NADH dependent KARI and ADH enzymes. With these modifications, strain SCP1N will generally exhibit higher isobutanol productivity, titer, and/or yield as compared to strain SCP1 and the parent strain GEVO1186.

As disclosed herein, one approach to balance the cofactor usage between glycolysis and the isobutanol pathway is the use of NADH dependent KARI and ADH. To accomplish this, the mitochondrially targeted NADH dependent KARI is cloned into pGV1817 replacing ScILV5 to generate pGV1817N. The NADH dependent ADH DmADH is cloned into pGV7001 replacing ScADH7 to generate pGV7001N.

GEVO1186, a diploid CEN.PK strain is then transformed with linear DNA from pGV1817N and from pGV7001N resulting in strain SCP1N. This combination of linear DNA contains genes coding for all five isobutanol pathway enzymes, the S. cerevisiae PDC1 promoter and terminator sequences for homologous integration into the PDC1 locus and the uracil marker for selection. This homologous replacement event results in the simultaneous integration of the isobutanol pathway along with the deletion of the PDC1 coding sequence. The NADH dependent ADH is not targeted to the mitochondria. All other pathway enzymes are targeted to the mitochondria. Pathway enzymes that are not natively localized to the mitochondria are fused to mitochondrial targeting sequences that direct them to the mitochondria. The transformed cells are plated onto selective medium without uracil and incubated at 30° C. for 3 days. After 3 days colonies are patched onto selective plates and these patches are used for colony PCR and RT-PCR to verify correct integration and transcription of the pathway genes. Three verified clones from each transformation are used to inoculate 3 mL overnight cultures in YPD. As controls parental strain GEVO1186 and the strain SCP1, which contains the imbalanced pathway, are used. These overnight cultures are used to inoculate 100 mL cultures in 1 L shake flasks. The cultures are harvested at an OD600 of 0.6-0.8. The cells are resuspended in 50 mL fresh YPD medium and the cultures are incubated in 250 mL shake flasks at 30° C., 250 rpm. 2 mL samples are taken at 24 and 48 hours after inoculation. The fermentation is ended after 48 hours. Samples are processed and analyzed by HPLC and GC.

### Example 10

### Mitochondrial Production of Isobutanol in a PDC-Minus S. cerevisiae with Cofactor Usage Balanced by NADH Dependent KARI and ADH, Wherein ALS, KARI, DHAD, and KIVD are Targeted to the Mitochondria

This example illustrates how mitochondrial production of isobutanol is achieved in PDC-minus yeast possessing NADH dependent KARI and ADH enzymes. With these modifications, strain SC1N will generally exhibit higher isobutanol productivity, titer, and/or yield as compared to strain SC1 and the parent strain GEVO7777, while at the same time, exhibiting reduced ethanol production.

As disclosed herein, one approach to balance the cofactor usage between glycolysis and the isobutanol pathway is the use of NADH dependent KARI and ADH. To accomplish this, a mitochondrially targeted NADH dependent KARI is cloned into pGV1817 replacing ILV5 to generate pGV1817N. A NADH dependent ADH is cloned into pGV7001 replacing ADH7 to generate pGV7001N.

GEVO7777, a haploid CEN.PK strain deleted for PDC5 and PDC6, is then transformed with linear DNA from pGV1817N and from pGV7001N resulting in strain SC1N. This combination of linear DNA contains genes coding for all five isobutanol pathway enzymes, the S. cerevisiae PDC1 promoter and terminator sequences for homologous integration into the PDC1 locus and the uracil marker for selection. The homologous replacement event results in the simultaneous integration of the isobutanol pathway along with the deletion of the PDC1 coding sequence. The NADH-dependent ADH is not targeted to the mitochondria. All other pathway enzymes are targeted to the mitochondria. Pathway enzymes that are not natively localized to the mitochondria are fused to mitochondrial targeting sequences that direct them to the mitochondria. The transformed cells are plated onto selective medium using ethanol as carbon source and lacking uracil and incubated at 30° C. for 3-4 days. After 3-4 days colonies are patched onto selective plates and these patches are used for colony PCR and RT-PCR to verify correct integration and transcription of the pathway genes. Three verified clones from each transformation are used to inoculate 3 mL overnight cultures in YPEthanol. As controls the parental strain GEVO7777 and the strain SC1, containing the imbalanced pathway, are used. These overnight cultures are used to inoculate 100 mL cultures in 1 L shake flasks. The cultures are harvested at an OD600 of 0.6-0.8. The cells are resuspended in 50 mL fresh YPD medium and the cultures are incubated in 250 mL shake flasks at 30° C., 250 rpm. 2 mL samples are taken at 24 and 48 hours after inoculation. The fermentation is ended after 48 hours. Samples are processed and analyzed by HPLC and GC.

### Example 11

### Mitochondrial Production of Isobutanol in C2-Independent PDC-Minus S. cerevisiae with Cofactor Usage Balanced by NADH Dependent KARI and ADH, Wherein ALS, KARI, DHAD, and KIVD are Targeted to the Mitochondria

This example illustrates how mitochondrial production of isobutanol is achieved in C2-independent PDC-minus yeast possessing NADH dependent KARI and ADH enzymes. With these modifications, strain c2i-SC1N will generally exhibit higher isobutanol productivity, titer, and/or yield as compared to strain SC1 and the parent strain GEVO1863.

As disclosed herein, one approach to balance the cofactor usage between glycolysis and the isobutanol pathway is the use of NADH-dependent KARI and ADH. To accomplish this, the mitochondrially targeted NADH-dependent KARI is cloned into pGV1817 replacing ILV5 to generate pGV1817N. The NADH dependent ADH is cloned into pGV7001 replacing ADH7 to generate pGV7001N.

S. cerevisiae which is deleted for all PDC genes (PDC1, PDC5 and PDC6) are dependent on C2-carbons such as ethanol or acetate for growth. Such a strain can be evolved to grow on glucose and hence be C2-independent (van Maris, A., et al., Applied and Environmental Microbiology, 2004, 70(1):159-166). GEVO1863 is a PDC-minus strain that has been evolved to grow on glucose. GEVO1863 is transformed with linear DNA from pGV1817N and from pGV7001N resulting in strain c2i-SC1N. This combination of linear DNA contains genes coding for all five isobutanol pathway enzymes, the S. cerevisiae PDC1 promoter and terminator sequences for homologous integration into the PDC1 locus and the uracil marker for selection. The homologous replacement event replaces the pdc1::ble allele. The NADH dependent ADH is not targeted to the mitochondria. All other pathway enzymes are targeted to the mitochondria. Pathway enzymes that are not natively localized to the mitochondria are fused to mitochondrial targeting sequences that direct them to the mitochondria. The transformed cells are plated onto selective medium lacking uracil and incubated at 30° C. for 3-4 days. After 3-4 days colonies are patched onto selective plates and these patches are used for colony PCR and RT-PCR to verify correct integration and transcription of the pathway genes. Three verified clones from each transformation are used to inoculate 3 mL overnight cultures in YPD. As a control the parental strain, GEVO1863 and the strain c2i-SC1 containing the imbalanced pathway, are used. These overnight cultures are used to inoculate 100 mL YPD cultures in 1 L shake flasks. The cultures are harvested at an OD600 of 0.6-0.8. The cells are resuspended in 50 mL fresh YPD medium and the cultures are incubated in 250 mL shake flasks at 30° C., 250 rpm. 2 mL samples are taken at 24 and 48 hours after inoculation. The fermentation is ended after 48 hours. Samples are processed and analyzed by HPLC and GC.

### Example 12

### Mitochondrial Production of Isobutanol in K. Marxianus with Cofactor Usage Partially Balanced by NADH Dependent ADH, Wherein ALS, KARI, DHAD, and KIVD are Targeted to the Mitochondria

This example illustrates how mitochondrial production is achieved in yeast with cofactor usage partially balanced by NADH dependent ADH, wherein ALS, KARI, DHAD, and KIVD are targeted to the mitochondria. With these modifications, strain AM will generally exhibit higher isobutanol productivity, titer, and/or yield as compared to strain A and the parent strain GEVO1947, while at the same time, exhibiting reduced ethanol production.

As disclosed herein, one approach to balance the cofactor usage between glycolysis and the isobutanol pathway is the use of NADH dependent KARI and ADH. To accomplish this, the NADH dependent ADH is cloned into pGV1834 replacing ADH7 to generate pGV1834N.

GEVO1947 is then transformed with linear DNA from pGV1832 and from pGV1834N resulting in strain AM. Each combination of linear DNA contains genes coding for all five isobutanol pathway enzymes, the K. marxianus PDC1 promoter and terminator sequences for homologous integration into the PDC1 locus and the uracil marker for selection. This homologous replacement event results in the simultaneous integration of the isobutanol pathway along with the deletion of the PDC1 coding sequence. The NADH dependent ADH is not targeted to the mitochondria. All other pathway enzymes are targeted to the mitochondria. Pathway enzymes that are not natively localized to the mitochondria are fused to mitochondrial targeting sequences that direct them to the mitochondria. The transformed cells are plated onto selective medium without uracil and incubated at 30° C. for 3 days. After 3 days colonies are patched onto selective plates and these patches are used for colony PCR and RT-PCR to verify correct integration and transcription of the pathway genes. Three verified clones from each transformation are used to inoculate 3 mL overnight cultures in YPD. As a control the parental strain, GEVO1947 and the strain A, containing the NADPH dependent isobutanol pathway, are used. These cultures are used to inoculate 100 mL cultures in 1 L shake flasks. These overnight cultures are harvested at an OD600 of 0.6-0.8. The cells are resuspended in 50 mL fresh YPD medium and the cultures are incubated in 250 mL shake flasks at 30° C., 250 rpm. 2 mL samples are taken at 24 and 48 hours after inoculation. The fermentation is ended after 48 hours. Samples are processed and analyzed by HPLC and GC.

### Example 13

### Mitochondrial Production of Isobutanol in K. Marxianus with Cofactor Usage Partially Balanced by NADH Dependent ADH, Wherein ALS, KARI, DHAD, and KIVD are Targeted to the Mitochondria

This example illustrates how mitochondrial production is achieved in yeast with cofactor usage partially balanced by NADH dependent ADH, wherein ALS, KARI, DHAD, and KIVD are targeted to the mitochondria. With these modifications, strain APM will generally exhibit higher isobutanol productivity, titer, and/or yield as compared to strain AP and the parent strain GEVO1947.

As disclosed herein, one approach to balance the cofactor usage between glycolysis and the isobutanol pathway is the use of NADH dependent KARI and ADH. To accomplish this, the NADH dependent ADH DmADH is cloned into pGV7001 replacing ScADH7 to generate pGV7001N.

GEVO1947 is then transformed with linear DNA from pGV1817 and from pGV7001N resulting in strain APM. This combination of linear DNA contains genes coding for all five isobutanol pathway enzymes, and the uracil marker for selection. The transformation leads to random (non-targeted) insertion of the isobutanol pathway into the K. marxianus genome. DmADH is not targeted to the mitochondria. All other pathway enzymes are targeted to the mitochondria. Pathway enzymes that are not natively localized to the mitochondria are fused to mitochondrial targeting sequences that direct them to the mitochondria. The transformed cells are plated onto selective medium without uracil and incubated at 30° C. for 3 days. After 3 days colonies are patched onto selective plates and these patches are used for colony PCR and RT-PCR to verify correct integration and transcription of the pathway genes. Three verified clones from each transformation are used to inoculate 3 mL overnight cultures in YPD. As a control GEVO1947 and the pathway containing the NADPH dependent isobutanol pathway, strain AP, are used. These cultures are used to inoculate 100 mL YPD cultures in 1 L shake flasks. These overnight cultures are harvested at an OD600 of 0.6-0.8. The cells are resuspended in 50 mL fresh YPD medium and the cultures are incubated in 250 mL shake flasks at 30° C., 250 rpm. 2 mL samples are taken at 24 and 48 hours after inoculation. The fermentation is ended after 48 hours. Samples are processed and analyzed by HPLC and GC.

### Example 14

### Mitochondrial Production of Isobutanol in S. Cerevisiae with Cofactor Usage Partially Balanced by NADH Dependent ADH, Wherein ALS, KARI, DHAD, and KIVD are Targeted to the Mitochondria

This example illustrates how mitochondrial production is achieved in yeast with cofactor usage partially balanced by NADH dependent ADH, wherein ALS, KARI, DHAD, and KIVD are targeted to the mitochondria. With these modifications, strain SCP1M will generally exhibit higher isobutanol productivity, titer, and/or yield as compared to strain SCP1 and the parent strain GEVO1186.

As disclosed herein, one approach to balance the cofactor usage between glycolysis and the isobutanol pathway is the use of NADH dependent KARI and ADH. To accomplish this, the NADH dependent ADH is cloned into pGV7001 replacing ADH7 to generate pGV7001N.

GEVO1186, a diploid CEN.PK strain, is then transformed with linear DNA from pGV1817 and from pGV7001N resulting in strain SCP1M. Each combination of linear DNA contains genes coding for all five isobutanol pathway enzymes, the S. cerevisiae PDC1 promoter and terminator sequences for homologous integration into the PDC1 locus and the uracil marker for selection. This homologous replacement event results in the simultaneous integration of the isobutanol pathway along with the deletion of the PDC1 coding sequence. The NADH dependent ADH is not targeted to the mitochondria. All other pathway enzymes are targeted to the mitochondria. Pathway enzymes that are not natively localized to the mitochondria are fused to mitochondrial targeting sequences that direct them to the mitochondria. The transformed cells are plated onto selective medium without uracil and incubated at 30° C. for 3 days. After 3 days colonies are patched onto selective plates and these patches are used for colony PCR and RT PCR to verify correct integration and transcription of the pathway genes. Three verified clones from each transformation are used to inoculate 3 mL overnight cultures in YPD. As controls the parental strain, GEVO1186, and the strain SCP1, containing the imbalanced pathway are used. These cultures are used to inoculate 100 mL cultures in 1 L shake flasks. These cultures are harvested at an OD600 of 0.6-0.8. The cells are resuspended in 50 mL fresh YPD medium and the cultures are incubated in 250 mL shake flasks at 30° C., 250 rpm. 2 mL samples are taken at 24 and 48 hours after inoculation. The fermentation is ended after 48 hours. Samples are processed and analyzed by HPLC and GC.

### Example 15

### Mitochondrial Production of Isobutanol in a PDC-Minus S. cerevisiae with Cofactor Usage Partially Balanced by NADH Dependent ADH, Wherein ALS, KARI, DHAD, and KIVD are Targeted to the Mitochondria

This example illustrates how mitochondrial production is achieved in yeast with cofactor usage partially balanced by NADH dependent ADH, wherein ALS, KARI, DHAD, and KIVD are targeted to the mitochondria. With these modifications, strain SC1M will generally exhibit higher isobutanol productivity, titer, and/or yield as compared to strain SC1 and the parent strain GEVO7777, while at the same time, exhibiting reduced ethanol production.

As disclosed herein, one approach to balance the cofactor usage between glycolysis and the isobutanol pathway is the use of NADH dependent KARI and ADH. To accomplish this, the NADH dependent ADH DmADH is cloned into pGV7001 replacing ADH7 to generate pGV7001N.

GEVO7777, a haploid S. cerevisiae strain in which is deleted for PDC5 and PDC6, is then transformed with linear DNA from pGV1817 and from pGV7001N resulting in strain SC1M. This combination of linear DNA contains genes coding for all five isobutanol pathway enzymes, the S. cerevisiae PDC1 promoter and terminator sequences for homologous integration into the PDC1 locus and the uracil marker for selection. This homologous replacement event results in the simultaneous insertion of the isobutanol pathway along with deletion of the PDC1 coding sequence. DmADH is not targeted to the mitochondria. All other pathway enzymes are targeted to the mitochondria. Pathway enzymes that are not natively localized to the mitochondria are fused to mitochondrial targeting sequences that direct them to the mitochondria. The transformed cells are plated onto selective medium using ethanol as carbon source and lacking uracil and incubated at 30° C. for 3-4 days. After 3-4 days colonies are patched onto selective plates and these patches are used for colony PCR and RT-PCR to verify correct integration and transcription of the pathway genes. Three verified clones from each transformation are used to inoculate 3 mL overnight cultures in YPEthanol. As controls the parental strain GEVO7777 and the strain SC1 containing the imbalanced pathway, are used. These overnight cultures are used to inoculate 100 mL cultures in 1 L YPEthanol shake flasks. The cultures are harvested at an OD600 of 0.6-0.8. The cells are resuspended in 50 mL fresh YPD medium and the cultures are incubated in 250 mL shake flasks at 30° C., 250 rpm. 2 mL samples are taken at 24 and 48 hours after inoculation. The fermentation is ended after 48 hours. Samples are processed and analyzed by HPLC and GC.

### Example 16

### Mitochondrial Production of Isobutanol in C2-Independent PDC-Minus S. cerevisiae with Cofactor Usage Partially Balanced by NADH Dependent ADH, Wherein ALS, KARI, DHAD, and KIVD are Targeted to the Mitochondria

This example illustrates how mitochondrial production is achieved in C2-independent PDC-minus yeast with cofactor usage partially balanced by NADH dependent ADH, wherein ALS, KARI, DHAD, and KIVD are targeted to the mitochondria. With these modifications, strain c2i-SC1M will generally exhibit higher isobutanol productivity, titer, and/or yield as compared to strain c2i-SC1 and the parent strain GEVO1863.

As disclosed herein, one approach to balance the cofactor usage between glycolysis and the isobutanol pathway is the use of NADH dependent KARI and ADH. To accomplish this, the NADH dependent ADH DmADH is cloned into pGV7001 replacing ScADH7 to generate pGV7001N.

S. cerevisiae which is deleted for all PDC genes (PDC1, PDC5 and PDC6) is dependent on C2-carbons such as ethanol or acetate for growth. Such a strain can be evolved to grow on glucose and hence be C2-independent (van Maris, A., et al., Applied and Environmental Microbiology, 2004, 70(1):159-166). GEVO1863 is a PDC-minus strain that has been evolved to grow on glucose. GEVO1863 is transformed with linear DNA from pGV1817 and from pGV7001N resulting in strain c2i-SC1M. This combination of linear DNA contains genes coding for all five isobutanol pathway enzymes, the S. cerevisiae PDC1 promoter and terminator sequences for homologous integration into the PDC1 locus and the uracil marker for selection. This homologous replacement event replaces the pdc1::ble allele with the isobutanol pathway. DmADH is not targeted to the mitochondria. All other pathway enzymes are targeted to the mitochondria. Pathway enzymes that are not natively localized to the mitochondria are fused to mitochondrial targeting sequences that direct them to the mitochondria. The transformed cells are plated onto selective medium lacking uracil and incubated at 30° C. for 3-4 days. After 3-4 days colonies are patched onto selective plates and these patches are used for colony PCR and RT-PCR to verify correct integration and transcription of the pathway genes. Three verified clones from each transformation are used to inoculate 3 mL overnight cultures in YPD. As controls, the parental GEVO1863 and the strain c2i-SC1 containing the imbalanced pathway, are used. These overnight cultures are used to inoculate 100 mL cultures in 1 L YPD shake flasks. The cultures are harvested at an OD600 of 0.6-0.8. The cells are resuspended in 50 mL fresh YPD medium and the cultures are incubated in 250 mL shake flasks at 30° C., 250 rpm. 2 mL samples are taken at 24 and 48 hours after inoculation. The fermentation is ended after 48 hours. Samples are processed and analyzed by HPLC and GC.

### Example 17

### Mitochondrial Production of Isobutanol in K. Marxianus with Cofactor Usage Partially Balanced by NADH Dependent KARI, Wherein ALS, KARI, DHAD, and KIVD are Targeted to the Mitochondria

This example illustrates how mitochondrial production is achieved in yeast with cofactor usage partially balanced by NADH dependent KARI, wherein ALS, KARI, DHAD, and KIVD are targeted to the mitochondria. With these modifications, strain AK will generally exhibit higher isobutanol productivity, titer, and/or yield as compared to strain A and the parent strain GEVO1947, while at the same time, exhibiting reduced ethanol production.

As disclosed herein, one approach to balance the cofactor usage between glycolysis and the isobutanol pathway is the use of NADH dependent KARI and ADH. To accomplish this, the mitochondrially targeted NADH dependent KARI is cloned into pGV1832 replacing ScILV5 to generate pGV1832N.

To construct strain AK, GEVO1947 is first transformed with linear DNA from pGV1832N and from pGV1834 resulting in strain AK. Each combination of linear DNA contains genes coding for all five isobutanol pathway enzymes, the K. marxianus PDC1 promoter and terminator sequences for homologous integration into the PDC1 locus and the uracil marker for selection. This homologous replacement event results in the simultaneous integration of the isobutanol pathway along with the deletion of the PDC1 coding sequence. ADH7 is not targeted to the mitochondria. All other pathway enzymes are targeted to the mitochondria. Pathway enzymes that are not natively localized to the mitochondria are fused to mitochondrial targeting sequences that direct them to the mitochondria. The transformed cells are plated onto selective medium without uracil and incubated at 30° C. for 3 days. After 3 days colonies are patched onto selective plates and these patches are used for colony PCR and RT-PCR to verify correct integration and transcription of the pathway genes. Three verified clones from each transformation are used to inoculate 3 mL overnight cultures in YPD. As controls GEVO1947 and the pathway containing the NADPH dependent isobutanol pathway, strain A, are used. These cultures are used to inoculate 100 mL YPD cultures in 1 L shake flasks. These overnight cultures are harvested at an OD600 of 0.6-0.8. The cells are resuspended in 50 mL fresh YPD medium and the cultures are incubated in 250 mL shake flasks at 30° C., 250 rpm. 2 mL samples are taken at 24 and 48 hours after inoculation. The fermentation is ended after 48 hours. Samples are processed and analyzed by HPLC and GC.

### Example 18

### Mitochondrial Production of Isobutanol in S. Cerevisiae with Cofactor Usage Partially Balanced by NADH Dependent KARI, Wherein ALS, KARI, DHAD, and KIVD are Targeted to the Mitochondria

This example illustrates how mitochondrial production is achieved in yeast with cofactor usage partially balanced by NADH dependent KARI, wherein ALS, KARI, DHAD, and KIVD are targeted to the mitochondria. With these modifications, strain SCP1K will generally exhibit higher isobutanol productivity, titer, and/or yield as compared to strain SCP1 and the parent strain GEVO1186.

As disclosed herein, one approach to balance the cofactor usage between glycolysis and the isobutanol pathway is the use of NADH dependent KARI and ADH. To accomplish this, the mitochondrially targeted NADH dependent KARI is cloned into pGV1817 replacing ScILV5 to generate pGV1817N.

GEVO1186 is then transformed with linear DNA from pGV1817N and from pGV7001 resulting in strain SCP1K. Each combination of linear DNA contains genes coding for all five isobutanol pathway enzymes, the S. cerevisiae PDC1 promoter and terminator sequences for homologous integration into the PDC1 locus and the uracil marker for selection. This homologous replacement event results in the simultaneous integration of the isobutanol pathway along with the deletion of the PDC1 coding sequence. ADH7 is not targeted to the mitochondria. All other pathway enzymes are targeted to the mitochondria. Pathway enzymes that are not natively localized to the mitochondria are fused to mitochondrial targeting sequences that direct them to the mitochondria. The transformed cells are plated onto selective medium without uracil and incubated at 30° C. for 3 days. After 3 days colonies are patched onto selective plates and these patches are used for colony PCR and RT-PCR to verify correct integration and transcription of the pathway genes. Three verified clones from each transformation are used to inoculate 3 mL overnight cultures in YPD. As controls GEVO1186 and the pathway containing the imbalanced pathway, strain SCP1, are used. These cultures are used to inoculate 100 mL YPD cultures in 1 L shake flasks. These cultures are harvested at an OD600 of 0.6-0.8. The cells are resuspended in 50 mL fresh YPD medium and the cultures are incubated in 250 mL shake flasks at 30° C., 250 rpm. 2 mL samples are taken at 24 and 48 hours after inoculation. The fermentation is ended after 48 hours. Samples are processed and analyzed by HPLC and GC.

### Example 19

### Mitochondrial Production of Isobutanol in K. Marxianus with Cofactor Usage Balanced and with ALS, KARI, DHAD, and KIVD Targeted to the Mitochondria

This example illustrates how mitochondrial production of isobutanol is achieved in yeast with balanced cofactor usage and with ALS, KARI, DHAD, and KIVD targeted to the mitochondria. With these modifications, strain APN will generally exhibit higher isobutanol productivity, titer, and/or yield as compared to strain AP and the parent strain GEVO1947.

To accomplish this, GEVO1947 is first transformed with linear DNA from pGV1817N and additional linear DNA from pGV7001N resulting in strain APN. This combination of linear DNA contains genes coding for all five isobutanol pathway enzymes, and the uracil marker for selection. The transformation leads to random (non-targeted) insertion of the isobutanol pathway into the K. marxianus genome. DmAdhp is targeted to its native compartment, the cytosol. All other pathway enzymes are targeted to the mitochondria. Pathway enzymes that are not natively localized to the mitochondria are fused to mitochondrial targeting sequences that direct them to the mitochondria. The transformed cells are plated onto selective medium without uracil and incubated at 30° C. for 3 days. After 3 days colonies are patched onto selective plates and these patches are used for colony PCR and RT-PCR to verify correct integration and transcription of the pathway genes. Three verified clones from each transformation are used to inoculate 3 mL overnight cultures in YPD. As controls GEVO1947 and strain AP are used. The overnight cultures are used to inoculate 100 mL YPD cultures in 1 L shake flasks. These cultures are harvested at an OD600 of 0.6-0.8. The cells are resuspended in 50 mL fresh YPD medium and the cultures are incubated in 250 mL shake flasks at 30° C., 250 rpm. 2 mL samples are taken at 24 and 48 hours after inoculation. The fermentation is ended after 48 hours. Samples are processed and analyzed by HPLC and GC.

### Example 20

### Mitochondrial Production of Isobutanol in K. Marxianus with Cofactor Usage Balanced, Wherein all Pathway Enzymes Targeted to the Mitochondria and PDC Deleted

This example illustrates how mitochondrial production of isobutanol is achieved in yeast with balanced cofactor usage, wherein all pathway enzymes are targeted to the mitochondria. With these modifications, strain A1N will generally exhibit higher isobutanol productivity, titer, and/or yield as compared to strain A1 and the parent strain GEVO1947. At the same time, strains A1N and A1 will generally exhibit reduced ethanol production as compared to the parent strain GEVO1947.

To accomplish this, GEVO1947 is first transformed with linear DNA from pGV1832N and additional linear DNA from pGV9834N resulting in strain A1N. The combination of linear DNA contains genes coding for all five isobutanol pathway enzymes, the K. marxianus PDC1 promoter and terminator sequences for homologous integration into the PDC1 locus and the uracil marker for selection. This homologous replacement event results in the simultaneous integration of the isobutanol pathway along with the deletion of the PDC1 coding sequence. All pathway enzymes are targeted to the mitochondria. Pathway enzymes that are not natively localized to the mitochondria are fused to mitochondrial targeting sequences that direct them to the mitochondria. The transformed cells are plated onto selective medium without uracil and incubated at 30° C. for 3 days. After 3 days colonies are patched onto selective plates and these patches are used for colony PCR and RT-PCR to verify correct integration and transcription of the pathway genes. Three verified clones from each transformation are used to inoculate 3 mL overnight cultures in YPD. As controls GEVO1947 and strain A1 are used. These cultures are used to inoculate 100 mL YPD cultures in 1 L shake flasks. These cultures are harvested at an OD600 of 0.6-0.8. The cells are resuspended in 50 mL fresh YPD medium and the cultures are incubated in 250 mL shake flasks at 30° C., 250 rpm. 2 mL samples are taken at 24 and 48 hours after inoculation. The fermentation is ended after 48 hours. Samples are processed and analyzed by HPLC and GC.

### Example 21

### Mitochondrial Production of Isobutanol in K. marxianus with Cofactor Usage Balanced and with all Pathway Enzymes Targeted to the Mitochondria

This example illustrates how mitochondrial production of isobutanol is achieved in yeast with balanced cofactor usage, wherein all pathway enzymes are targeted to the mitochondria. With these modifications, strain A1PN will generally exhibit higher isobutanol productivity, titer, and/or yield as compared to strain A1P and the parent strain GEVO1947.

To accomplish this, GEVO1947 is first transformed with linear DNA from pGV1817N and additional linear DNA from pGV7101N resulting in strain A1PN. This combination of linear DNA contains genes coding for all five isobutanol pathway enzymes, and the uracil marker for selection. The transformation leads to random (non-targeted) integration of the isobutanol pathway into the K. marxianus genome. All pathway enzymes are targeted to the mitochondria. Pathway enzymes that are not natively localized to the mitochondria are fused to mitochondrial targeting sequences that direct them to the mitochondria. The transformed cells are plated onto selective medium without uracil and incubated at 30° C. for 3 days. After 3 days colonies are patched onto selective plates and these patches are used for colony PCR and RT-PCR to verify correct integration and transcription of the pathway genes. Three verified clones from each transformation are used to inoculate 3 mL overnight cultures in YPD. As controls GEVO1947 and strain A1P are used. These cultures are used to inoculate 100 mL YPD cultures in 1 L shake flasks. These cultures are harvested at an OD600 of 0.6-0.8. The cells are resuspended in 50 mL fresh YPD medium and the cultures are incubated in 250 mL shake flasks at 30° C., 250 rpm. 2 mL samples are taken at 24 and 48 hours after inoculation. The fermentation is ended after 48 hours. Samples are processed and analyzed by HPLC and GC.

### Example 22

### Mitochondrial Production of Isobutanol in S. cerevisiae with Cofactor Usage Balanced and with all Pathway Enzymes Targeted to the Mitochondria

This example illustrates how mitochondrial production of isobutanol is achieved in yeast with balanced cofactor usage, wherein all pathway enzymes are targeted to the mitochondria. With these modifications, strain SCPA1N will generally exhibit higher isobutanol productivity, titer, and/or yield as compared to strain SCPA1 and the parent strain GEVO1186.

To accomplish this, GEVO1186, a diploid CEN.PK strain, is first transformed with linear DNA from pGV1817N and additional linear DNA from either pGV7101N resulting in strain SCPA1N. Each combination of linear DNA contains genes coding for all five isobutanol pathway enzymes, the S. cerevisiae PDC1 promoter and terminator sequences for homologous integration into the PDC1 locus and the uracil marker for selection. This homologous replacement event results in the simultaneous integration of the isobutanol pathway along with the deletion of the PDC1 coding sequence. All pathway enzymes are targeted to the mitochondria. Isobutanol pathway enzymes that are not natively localized to the mitochondria are fused to mitochondrial targeting sequences that direct them to the mitochondria. The transformed cells are plated onto selective medium without uracil and incubated at 30° C. for 3-4 days. After 3-4 days colonies are patched onto selective plates and these patches are used for colony PCR and RT-PCR to verify correct integration and transcription of the pathway genes. Three verified clones from each transformation are used to inoculate 3 mL overnight cultures in YPD. As controls GEVO1186 and strain SCPA1 are used. These overnight cultures are used to inoculate 100 mL YPD cultures in 1 L shake flasks. The cultures are harvested at an OD600 of 0.6-0.8. The cells are resuspended in 50 mL fresh YPD medium and the cultures are incubated in 250 mL shake flasks at 30° C., 250 rpm. 2 mL samples are taken at 24 and 48 hours after inoculation. The fermentation is ended after 48 hours. Samples are processed and analyzed by HPLC and GC.

### Example 23

### Mitochondrial Production of Isobutanol in a PDC-Minus S. cerevisiae with Cofactor Usage Balanced and with all Pathway Enzymes Targeted to the Mitochondria

This example illustrates how mitochondrial production of isobutanol is achieved in PDC-minus yeast with balanced cofactor usage, wherein all pathway enzymes are targeted to the mitochondria. With these modifications, strain SCA1N will generally exhibit higher isobutanol productivity, titer, and/or yield as compared to strain SCA1 and the parent strain GEVO7777, while at the same time, exhibiting reduced ethanol production.

To accomplish this, GEVO7777, which is deleted for PDC5 and PDC6, is first transformed with linear DNA from pGV1817N and additional linear DNA from pGV7101N resulting in strain SCA1N. The combination of linear DNA contains genes coding for all five isobutanol pathway enzymes, the S. cerevisiae PDC1 promoter and terminator sequences for homologous integration into the PDC1 locus and the uracil marker for selection. This homologous replacement event results in the simultaneous integration of the isobutanol pathway along with the deletion of the PDC1 coding sequence. All pathway enzymes are targeted to the mitochondria. Pathway enzymes that are not natively localized to the mitochondria are fused to mitochondrial targeting sequences that direct them to the mitochondria. The transformed cells are plated onto selective medium using ethanol as carbon source and lacking uracil and incubated at 30° C. for 3-4 days. After 3-4 days colonies are patched onto selective plates and these patches are used for colony PCR and RT-PCR to verify correct integration and transcription of the pathway genes. Three verified clones from each transformation are used to inoculate 3 mL overnight cultures in YPEthanol. As a control GEVO7777 and strain SCA1 are used. These overnight cultures are used to inoculate 100 mL YPEthanol cultures in 1 L shake flasks. The cultures are harvested at an OD600 of 0.6-0.8. The cells are resuspended in 50 mL fresh YPD medium and the cultures are incubated in 250 mL shake flasks at 30° C., 250 rpm. 2 mL samples are taken at 24 and 48 hours after inoculation. The fermentation is ended after 48 hours. Samples are processed and analyzed by HPLC and GC.

### Example 24

### Mitochondrial Production of Isobutanol in C2-Independent PDC-Minus S. Cerevisiae with Cofactor Usage Balanced and with all Pathway Enzymes Targeted to the Mitochondria

This example illustrates how mitochondrial production of isobutanol is achieved in C2-independent PDC-minus yeast with balanced cofactor usage, wherein all pathway enzymes are targeted to the mitochondria. With these modifications, strain c2i-SCA1N will generally exhibit higher isobutanol productivity, titer, and/or yield as compared to strain c2i-SCA1 and the parent strain GEVO1863.

S. cerevisiae which is deleted for all PDC genes (PDC1, PDC5 and PDC6) is dependent on C2-carbons such as ethanol or acetate for growth. Such a strain can be evolved to grow on glucose and hence be C2-independent (van Maris, A., et al., Applied and Environmental Microbiology, 2004, 70(1):159-166). GEVO1863 is a Pdc-minus strain that has been evolved for growth on glucose. GEVO1863 is transformed with linear DNA from pGV1817N and additional linear DNA from pGV7101N resulting in strain c2i-SCA1N. Each combination of linear DNA contains genes coding for all five isobutanol pathway enzymes, the S. cerevisiae PDC1 promoter and terminator sequences for homologous integration into the PDC1 locus and the uracil marker for selection. This homologous replacement event simultaneous integrates the isobutanol pathway along with replacing the pdc1::ble allele. All pathway enzymes are targeted to the mitochondria. Pathway enzymes that are not natively localized to the mitochondria are fused to mitochondrial targeting sequences that direct them to the mitochondria. The transformed cells are plated onto selective medium lacking uracil and incubated at 30° C. for 3-4 days. After 3-4 days colonies are patched onto selective plates and these patches are used for colony PCR and RT-PCR to verify correct integration and transcription of the pathway genes. Three verified clones from each transformation are used to inoculate 3 mL overnight cultures in YPD. As controls GEVO1863 and c2i-SCA1 are used. These overnight cultures are used to inoculate 100 mL YPD cultures in 1 L shake flasks. The cultures are harvested at an OD600 of 0.6-0.8. The cells are resuspended in 50 mL fresh YPD medium and the cultures are incubated in 250 mL shake flasks at 30° C., 250 rpm. 2 mL samples are taken at 24 and 48 hours after inoculation. The fermentation is ended after 48 hours. Samples are processed and analyzed by HPLC and GC.

### Example 25

### Anaerobic Mitochondrial Production of Isobutanol in S. Cerevisiae with Cofactor Usage Balanced by NADH Dependent KARI and ADH, Wherein ALS, KARI, DHAD, and KIVD are Targeted to the Mitochondria

This example illustrates how anaerobic mitochondrial production of isobutanol is achieved in yeast possessing NADH dependent KARI and ADH enzymes. With these modifications, strain SCP1N will generally exhibit higher isobutanol productivity, titer, and/or yield under anaerobic conditions as compared to strain SCP1 and the parent strain GEVO1186.

As disclosed herein, one approach to balance the cofactor usage between glycolysis and the isobutanol pathway is the use of NADH dependent KARI and ADH. To accomplish this, the mitochondrially targeted NADH dependent KARI is cloned into pGV1817 replacing ScILV5 to generate pGV1817N. The NADH dependent ADH DmADH is cloned into pGV7001 replacing ScADH7 to generate pGV7001N.

GEVO1186, a diploid CEN.PK strain is then transformed with linear DNA from pGV1817N and from pGV7001N resulting in strain SCP1N. This combination of linear DNA contains genes coding for all five isobutanol pathway enzymes, the S. cerevisiae PDC1 promoter and terminator sequences for homologous integration into the PDC1 locus and the uracil marker for selection. This homologous replacement event results in the simultaneous integration of the isobutanol pathway along with the deletion of the PDC1 coding sequence. The NADH dependent ADH is not targeted to the mitochondria. All other pathway enzymes are targeted to the mitochondria. Pathway enzymes that are not natively localized to the mitochondria are fused to mitochondrial targeting sequences that direct them to the mitochondria. The transformed cells are plated onto selective medium without uracil and incubated at 30° C. for 3 days. After 3 days colonies are patched onto selective plates and these patches are used for colony PCR and RT-PCR to verify correct integration and transcription of the pathway genes. Three verified clones from each transformation are used to inoculate 3 mL overnight cultures in YPD. As controls parental strain GEVO1186 and the strain SCP1, which contains the imbalanced pathway, are used. These overnight cultures are used to inoculate 100 mL cultures in 1 L shake flasks. The cultures are harvested at an OD600 of 0.6-0.8. The cells are resuspended in 20 mL fresh YPD medium and the cultures are incubated in 100 mL stoppered serum bottles at 30° C. 2 mL samples are taken at 24 and 48 hours after inoculation. The fermentation is ended after 48 hours. Samples are processed and analyzed by HPLC and GC.

### Example 26

### Mitochondrial Production of Isobutanol with Cofactor Usage Balanced by Malate Bypass, Wherein ALS, KARI, DHAD, and KIVD are Targeted to the Mitochondria

This example illustrates how mitochondrial production of isobutanol is achieved in yeast with cofactor usage balanced by malate bypass, wherein ALS, KARI, DHAD, and KIVD are targeted to the mitochondria. With these modifications, malate-bypass containing strains will generally exhibit higher isobutanol productivity, titer, and/or yield as compared to their respective parent strains.

As disclosed herein, one approach to balance the cofactor usage for the production of isobutanol from glucose is the use of a malate bypass in which the NADH generated from glycolysis is consumed in the production of malate from phosphoenol pyruvate. Malate is then transported into the mitochondria and used to generate NADPH and pyruvate. To introduce this bypass the following genes are overexpressed in the yeast host: PCK1, MDH2, DIC1 and MAE1. pGV8000 is a plasmid in which these genes are expressed using strong constitutive promoters. The plasmid also expresses the hygromycin resistance gene, hph. Downstream of the pathway genes, pGV8000 carries a sequence which consists of, from 5′ to 3′, S. cerevisiae PDC6 terminator, a unique restriction site (HpaI), and the PDC6 promoter. pGV8000 is linearized by digestion with HpaI and is transformed into K. marxianus strains A, AM, AK, AP, and APM for random integration into the chromosome. This results in the K. marxianus strains AMB, AMMB, AKMB, APMB, and APMMB respectively. This linearized pGV8000, when transformed into S. cerevisiae homologously replaces the PDC6 locus of S. cerevisiae. The linearized pGV8000 is transformed into strains SCP1, SCP1M, SCP1K, SC1, SC1M, c2i-SC1, and c2i-SC1M resulting in strains SCP1 MB, SCP1MMB, SCP1KMB, SC1 MB, SC1MMB, c2i-SC1 MB, and c2i-SC1MMB respectively. The transformed cells are plated onto YPD medium supplemented with hygromycin. Except in the case of SC1, SC1 MB, SC1MMB and SC1M strains, YPEthanol supplemented with hygromycin is used. The plates are incubated at 30° C. for 3-4 days. After 3-4 days colonies are patched onto selective plates and these patches are used for colony PCR and RT-PCR to verify correct integration and transcription of the malate bypass genes, respectively. Three verified clones from each transformation are used to inoculate 3 mL overnight cultures in YPD or in YPEthanol for the strains SC1, SC1 MB, SC1MMB and SC1M. The parent strains are used as controls. These overnight cultures are used to inoculate 100 mL cultures in 1 L YPD or YPEthanol shake flasks. These cultures are harvested at an OD600 of 0.6-0.8. The cells are resuspended in 50 mL fresh YPD medium and the cultures are incubated in 250 mL shake flasks at 30° C., 250 rpm. 2 mL samples are taken at 24 and 48 hours after inoculation. The fermentation is ended after 48 hours. Samples are processed and analyzed by HPLC and GC.

### Example 27

### Mitochondrial Production of Isobutanol with Cofactor Usage Balanced by Malate Bypass, Wherein all Isobutanol Pathway Enzymes are Targeted to the Mitochondria

This example illustrates how mitochondrial production of isobutanol is achieved in yeast with cofactor usage balanced by malate bypass, wherein all isobutanol pathway enzymes are targeted to the mitochondria. With these modifications, malate-bypass containing strains will generally exhibit higher isobutanol productivity, titer, and/or yield as compared to their respective parent strains.

As disclosed herein, one approach to balance the cofactor usage for the production of isobutanol from glucose is the use of a malate bypass in which the NADH generated from glycolysis is consumed in the production of malate from phosphoenol pyruvate. Malate is then transported into the mitochondria and used to generate NADPH and pyruvate. To introduce this bypass the following genes are overexpressed in the yeast host: PCK1, MDH2, DIC1 and MAE1. pGV8000 is a plasmid in which these genes are expressed using strong constitutive promoters. The plasmid also expresses the hygromycin resistance gene, hph. Downstream of the pathway genes, pGV8000 carries a sequence which consists of, from 5′ to 3′, S. cerevisiae PDC6 terminator, a unique restriction site (HpaI), and the PDC6 promoter. pGV8000 is linearized by digestion with HpaI and is transformed into K. marxianus strains A1 and A1P for random integration into the chromosome. This results in the K. marxianus strains A1MB, and A1PMB, respectively. This linearized pGV8000, when transformed into S. cerevisiae homologously replaces the PDC6 locus of S. cerevisiae. The linearized pGV8000 is transformed into strains SCPA1, SCA1, and c2i-SCA1 resulting in strains SCPA1MB, SCA1MB, and c2i-SCA1MB respectively. The transformed cells are plated onto YPD medium supplemented with hygromycin. Except in the case of strains SCA1 and SCA1MB, for which YPEthanol supplemented with hygromycin is used. The plates are incubated at 30° C. for 3-4 days. After 3-4 days colonies are patched onto selective plates and these patches are used for colony PCR and RT-PCR to verify correct integration and transcription of the malate bypass genes, respectively. Three verified clones from each transformation are used to inoculate 3 mL overnight cultures in YPD or in YPEthanol for the strains SCA1 and SCA1MB. The parent strains are used as controls. These overnight cultures are used to inoculate 100 mL cultures in 1 L YPD or YPEthanol shake flasks. These cultures are harvested at an OD600 of 0.6-0.8. The cells are resuspended in 50 mL fresh YPD medium and the cultures are incubated in 250 mL shake flasks at 30° C., 250 rpm. 2 mL samples are taken at 24 and 48 hours after inoculation. The fermentation is ended after 48 hours. Samples are processed and analyzed by HPLC and GC. The malate-bypass containing strains show higher isobutanol productivity, titer and yield when compared to their respective parent strains.

### Example 28

### Mitochondrial Production of Isobutanol with Cofactor Balance Through Overexpression of a Fungal Transhydrogenase, Wherein ALS, KARI, DHAD, and KIVD are Targeted to the Mitochondria

This example illustrates how mitochondrial production of isobutanol is achieved in yeast with cofactor usage balanced via the overexpression of a fungal transhydrogenase, wherein ALS, KARI, DHAD, and KIVD are targeted to the mitochondria. With these modifications, the transhydrogenase containing strains will generally exhibit higher isobutanol productivity, titer, and/or yield as compared to their respective parent strains.

As disclosed herein, one approach to balance the cofactor usage between glycolysis and the isobutanol pathway is the use of a transhydrogenase in which the NADH generated from glycolysis is transferred into the mitochondria by the acetaldehyde/ethanol shuttle. In the mitochondria the NADH is converted into NADPH by a transhydrogenase which is integrated into the mitochondrial inner membrane. pGV9000 containing the transhydrogenase gene from Neurospora crassa is linearized by digestion with HpaI and transformed into K. marxianus strains A, AM, AK, AP, and APM for random integration into the chromosome. This results in the K. marxianus strains ATH, AMTH, AKTH, APTH, and APMTH respectively. This linearized pGV9000, when transformed into S. cerevisiae will homologously replace the PDC6 locus of S. cerevisiae. The linearized pGV9000 is transformed into strains SCP1, SCP1M, SCP1K, SC1, SC1M, c2i-SC1, and c2i-SC1M resulting in strains SCP1TH, SCP1MTH, SCP1KTH, SC1TH, SC1MTH, c2i-SC1TH, and c2i-SC1MTH respectively. The transformed strains are plated onto YPD medium supplemented with hygromycin. In the case of SC1, SC1TH, SC1MTH and SC1M strains, YPEthanol supplemented with hygromycin is used. All plates are incubated at 30° C. for 3-4 days. After 3-4 days colonies are patched onto selective plates and these patches are used for colony PCR and RT-PCR to verify correct integration and transcription of the transhydrogenase genes, respectively. Three verified clones from each transformation are used to inoculate 3 mL overnight cultures in YPD or YPEthanol for the Saccharomyces strains SC1, SC1TH, SC1MTH and SC1M. The parent strains are used as control. These overnight cultures are used to inoculate 100 mL cultures in 1 L shake flasks. These cultures are harvested at an OD600 of 0.6-0.8. The cells are resuspended in 50 mL fresh YPD medium and the cultures are incubated in 250 mL shake flasks at 30° C., 250 rpm. 2 mL samples are taken at 24 and 48 hours after inoculation. The fermentation is ended after 48 hours. Samples are processed and analyzed by HPLC and GC.

### Example 29

### Mitochondrial Production of Isobutanol with Cofactor Balance Through Overexpression of a Fungal Transhydrogenase, Wherein all Isobutanol Pathway Enzymes are Targeted to the Mitochondria

This example illustrates how mitochondrial production of isobutanol is achieved in yeast with cofactor usage balanced via the overexpression of a fungal transhydrogenase, wherein all isobutanol pathway enzymes are targeted to the mitochondria. With these modifications, the transhydrogenase containing strains will generally exhibit higher isobutanol productivity, titer, and/or yield as compared to their respective parent strains.

As disclosed herein, one approach to balance the cofactor usage between glycolysis and the isobutanol pathway is the use of a transhydrogenase in which the NADH generated from glycolysis is transferred into the mitochondria by the acetaldehyde/ethanol shuttle. In the mitochondria the NADH is converted into NADPH by a transhydrogenase which is integrated into the mitochondrial inner membrane. pGV9000 containing the transhydrogenase gene from Neurospora crassa is linearized by digestion with HpaI and transformed into K. marxianus strains A1 and A1P for random insertion into the chromosome. This results in the K. marxianus strains A1TH and A1PTH, respectively. This linearized pGV9000, when transformed into S. cerevisiae will homologously replace the PDC6 locus of S. cerevisiae. The linearized pGV9000 is transformed into strains SCPA1, SCA1, and c2i-SCA1, resulting in strains SCPA1TH, SCA1TH, and c2i-SCA1TH, respectively. The transformed strains are plated onto YPD medium supplemented with hygromycin. In the case of SCA1 and SCA1TH strains, YPEthanol supplemented with hygromycin is used. All plates are incubated at 30° C. for 3-4 days. After 3-4 days colonies are patched onto selective plates and these patches are used for colony PCR and RT-PCR to verify correct integration and transcription of the transhydrogenase genes, respectively. Three verified clones from each transformation are used to inoculate 3 mL overnight cultures in YPD or YPEthanol for the Saccharomyces strains SC1TH and SC1MTH. The parent strains are used as control. These overnight cultures are used to inoculate 100 mL cultures in 1 L shake flasks. These cultures are harvested at an OD600 of 0.6-0.8. The cells are resuspended in 50 mL fresh YPD medium and the cultures are incubated in 250 mL shake flasks at 30° C., 250 rpm. 2 mL samples are taken at 24 and 48 hours after inoculation. The fermentation is ended after 48 hours. Samples are processed and analyzed by HPLC and GC.

### Example 30

### Anaerobic Mitochondrial Production of Isobutanol with Cofactor Usage Balanced by Malate Bypass, Wherein ALS, KARI, DHAD, and KIVD are Targeted to the Mitochondria

This example illustrates how anaerobic mitochondrial production of isobutanol is achieved in yeast with cofactor usage balanced by malate bypass, wherein ALS, KARI, DHAD, and KIVD are targeted to the mitochondria. With these modifications, malate-bypass containing strains will generally exhibit higher isobutanol productivity, titer, and/or yield under anaerobic conditions as compared to their respective parent strains.

As disclosed herein, one approach to balance the cofactor usage for the production of isobutanol from glucose is the use of a malate bypass in which the NADH generated from glycolysis is consumed in the production of malate from phosphoenol pyruvate. Malate is then transported into the mitochondria and used to generate NADPH and pyruvate. To introduce this bypass the following genes are overexpressed in the yeast host: PCK1, MDH2, DIC1 and MAE1. pGV8000 is a plasmid in which these genes are expressed using strong constitutive promoters. The plasmid also expresses the hygromycin resistance gene, hph. Downstream of the pathway genes, pGV8000 carries a sequence which consists of, from 5′ to 3′, S. cerevisiae PDC6 terminator, a unique restriction site (HpaI), and the PDC6 promoter. pGV8000 is linearized by digestion with HpaI and is transformed into K. marxianus strains A, AM, AK, AP, and APM for random integration into the chromosome. This results in the K. marxianus strains AMB, AMMB, AKMB, APMB, and APMMB respectively. This linearized pGV8000, when transformed into S. cerevisiae homologously replaces the PDC6 locus of S. cerevisiae. The linearized pGV8000 is transformed into strains SCP1, SCP1M, SCP1K, SC1, SC1M, c2i-SC1, and c2i-SC1M resulting in strains SCP1 MB, SCP1MMB, SCP1KMB, SC1 MB, SC1MMB, c2i-SC1 MB, and c2i-SC1MMB respectively. The transformed cells are plated onto YPD medium supplemented with hygromycin. Except in the case of SC1, SC1 MB, SC1MMB and SC1M strains, YPEthanol supplemented with hygromycin is used. The plates are incubated at 30° C. for 3-4 days. After 3-4 days colonies are patched onto selective plates and these patches are used for colony PCR and RT-PCR to verify correct integration and transcription of the malate bypass genes, respectively. Three verified clones from each transformation are used to inoculate 3 mL overnight cultures in YPD or in YPEthanol for the strains SC1, SC1 MB, SC1MMB and SC1M. The parent strains are used as controls. These overnight cultures are used to inoculate 100 mL cultures in 1 L YPD or YPEthanol shake flasks. These cultures are harvested at an OD600 of 0.6-0.8. The cells are resuspended in 20 mL fresh YPD medium and the cultures are incubated in 100 mL stoppered serum bottles at 30° C., 250 rpm. 2 mL samples are taken at 24 and 48 hours after inoculation. The fermentation is ended after 48 hours. Samples are processed and analyzed by HPLC and GC.

### Example 31

### Anaerobic Mitochondrial Production of Isobutanol with Cofactor Balance through overexpression of a fungal transhydrogenase, wherein ALS, KARI, DHAD, and KIVD are Targeted to the Mitochondria

This example illustrates how anaerobic mitochondrial production of isobutanol is achieved in yeast with cofactor usage balanced via the overexpression of a fungal transhydrogenase, wherein ALS, KARI, DHAD, and KIVD are targeted to the mitochondria. With these modifications, the transhydrogenase containing strains will generally exhibit higher anaerobic isobutanol productivity, titer, and/or yield as compared to their respective parent strains.

As disclosed herein, one approach to balance the cofactor usage between glycolysis and the isobutanol pathway is the use of a transhydrogenase in which the NADH generated from glycolysis is transferred into the mitochondria by the acetaldehyde/ethanol shuttle. In the mitochondria the NADH is converted into NADPH by a transhydrogenase which is integrated into the mitochondrial inner membrane. pGV9000 containing the transhydrogenase gene from Neurospora crassa is linearized by digestion with HpaI and transformed into K. marxianus strains A, AM, AK, AP, and APM for random integration into the chromosome. This results in the K. marxianus strains ATH, AMTH, AKTH, APTH, and APMTH respectively. This linearized pGV9000, when transformed into S. cerevisiae will homologously replace the PDC6 locus of S. cerevisiae. The linearized pGV9000 is transformed into strains SCP1, SCP1M, SCP1K, SC1, SC1M, c2i-SC1, and c2i-SC1M resulting in strains SCP1TH, SCP1MTH, SCP1KTH, Sc1TH, Sc1MTH, c2i-SC1TH, and c2i-SC1MTH respectively. The transformed strains are plated onto YPD medium supplemented with hygromycin. In the case of SC1, SC1TH, SC1MTH and SC1M strains, YPEthanol supplemented with hygromycin is used. All plates are incubated at 30° C. for 3-4 days. After 3-4 days colonies are patched onto selective plates and these patches are used for colony PCR and RT-PCR to verify correct integration and transcription of the transhydrogenase genes, respectively. Three verified clones from each transformation are used to inoculate 3 mL overnight cultures in YPD or YPEthanol for the Saccharomyces strains SC1, SC1TH, SC1MTH and SC1M. The parent strains are used as control. These overnight cultures are used to inoculate 100 mL cultures in 1 L shake flasks. These cultures are harvested at an OD600 of 0.6-0.8. The cells are resuspended in 20 mL fresh YPD medium and the cultures are incubated in 100 mL stoppered serum bottles at 30° C., 250 rpm. 2 mL samples are taken at 24 and 48 hours after inoculation. The fermentation is ended after 48 hours. Samples are processed and analyzed by HPLC and GC.

### Example 32

### Comparison of Strains GEVO1820, GEVO2062 and GEVO2072

The goal of this experiment was to integrate mitochondrially targeted isobutanol pathways into the PDC1 locus of a diploid S. cerevisiae and to evaluate the ability of these strains to produce isobutanol in comparison to a strain expressing a cytosolically expressed isobutanol pathway. Mitochondrial targeting peptide sequences were added to the protein sequences of ALS, KIVD, and Aro10 in order to localize these proteins in the mitochondria. Because isobutyraldehyde is expected to be membrane permeable, it should be available as a substrate for the Adh7 enzyme overexpressed in the cytosol.

This example describes the comparison of two strains containing mitochondrially targeted isobutanol pathways: GEVO2062 and GEVO2072 (Table 1) with a strain containing a cytosolic isobutanol pathway. GEVO2072 and GEVO2062 are PDC positive diploid strains containing a mitochondrially targeted acetolactate synthase from B. subtilis (alsS), and a cytosolic S. cerevisiae alcohol dehydrogenase (ADH7). In addition, GEVO2062 express a mitochondrially targeted Aro10 protein; while GEVO2072 expresses a mitochondrially targeted KIVD from Lactococcus lactis. GEVO2072 and GEVO2062 were constructed by integrating a set of genes from pGV1875 (SEQ ID NO: 92) and pGV1876 respectively (SEQ ID NO: 97) into one allele of the native PDC1 locus. The integrated genes in GEVO2072 encode the B. subtilis AlsS protein with 31 amino acids of the mitochondrial targeting leader-sequence from S. cerevisiae Cox4 protein, L. lactis 2-keto-acid decarboxylase (KIVD) with the carboxy-terminal mitochondrial targeting sequence from the S. cerevisiae Hmi1 protein, and S. cerevisiae ADH7, which is expressed in the cytosol. The integrated genes in GEVO2062 encode the B. subtilis ALS protein with 25 amino acids of the mitochondrial targeting leader-sequence from S. cerevisiae Cox4 protein, the S. cerevisiae Aro10 protein with the carboxy-terminal mitochondrial targeting sequence from the S. cerevisiae Hmi1 protein, and S. cerevisiae ADH7, which is expressed in the cytosol.

The third strain in this example GEVO1820 contains a completely cytosolically expressed isobutanol pathway. GEVO1820 was generated by transforming the diploid wild-type strain GEVO1186 with a 9.2 kb HpaI fragment from pGV1733. Ura+ transformants contained a homologous replacement of one of the two PDC6 alleles with a copy of the LlkivD2, ScADH7, and ScURA3 genes to generate strain GEVO1802. Next GEVO1802 was transformed with a 9.5 kb NruI fragment from pGV1731. Leu+ transformants contained a homologous replacement of one of the two PDC5 alleles with a copy of the EcilvCΔN (SEQ ID NO: 157), EcilvDΔNcoKl (SEQ ID NO: 158), and ScLEU2 genes to generate strain GEVO1805. Finally GEVO1805 was transformed with a 6.7 kb linear fragment from pGV1730. Trp+ transformants contained a homologous replacement of one of the two PDC1 alleles with a copy of the BsalsS2 gene (SEQ ID NO: 151) coding for BsAlsS2 (SEQ ID NO: 152) expressed from the P_{ScCUP1} promoter and the TRP1 gene; the resulting strain is GEVO1820.

For aerobic fermentations, precultures were inoculated using cells from a fresh plate that were resuspended in 1 mL of YPD medium. These cell suspensions were then used to inoculate 50 mL of YPD medium in 250 mL baffled shake flasks; these preculture flasks were incubated at 30° C. in an orbital shaker at 250 rpm until the cell population reached mid-to late-log phase. To start the fermentation, cells were harvested from the preculture flasks and were used to inoculate 25 mL of fresh YPD (50 g-glucose L⁻¹) medium to a final OD₆₀₀ of 1.6-1.9. The 25 mL fermentation cultures were incubated in 250 mL non-baffled flasks at 30° C. in an orbital shaker at 250 rpm. Samples (2 mL) were taken at 0, 9, 23, 31, 51, and 61 hours and cells were removed from the samples by centrifugation at ≧14,000×g for 10 min in a microcentrifuge. The supernatants from the samples were collected and the glucose concentration was measured at each time point on the YSI 2700 Select (YSI Life Sciences, Yellow Springs, Ohio, USA) according to manufacturer's protocols. The remaining supernatant volume was stored at 4° C. until analysis by Gas Chromatography and/or High Performance Liquid Chromatography as described above.

Because of the fast glucose consumption rate of these strain, glucose was added throughout the time course. For all three strains, the fermentation cultures were fed 83 g/L glucose at 9 h, 30 g/L at 23 h, 121 g/L at 31 h. Additionally, the cultures of GEVO2062 and GEVO2072 were fed 60 g/L glucose at 51 h. These concentrations are volume corrected to account for the significant dilution of the cultures by the glucose feed. The concentrations refer to the start volume of the cultures.

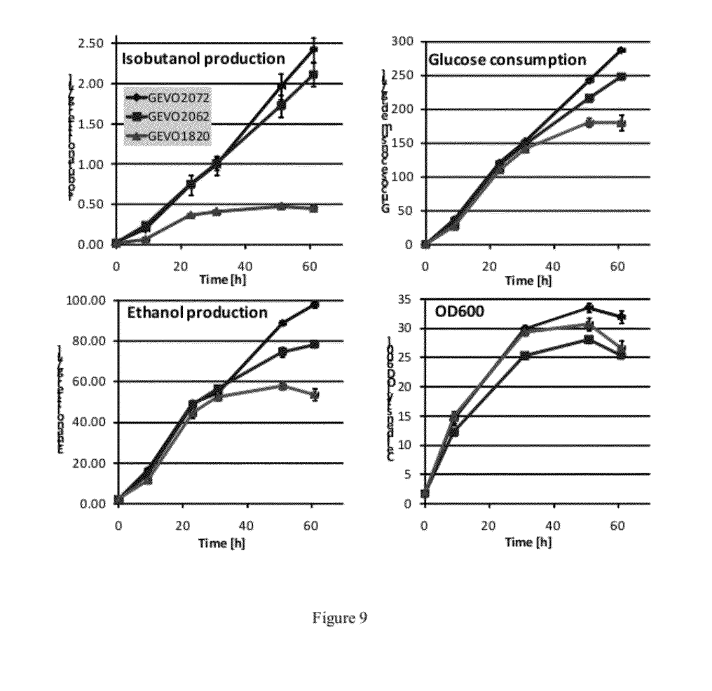

The differences observed between GEVO2062 and GEVO2072 during fermentation were minor GEVO2072 reached 15% higher titer than GEVO2062 (Table 4, FIG. 9). GEVO2072 grew to a 25% higher cell density and therefore the specific productivity of this strain was about 10% lower than that of GEVO2062. The yield was the same for both mitochondrial strains. GEVO2072 differs from GEVO2062 by the mitochondrial targeting sequence fused to ALS. GEVO2062 has a 25 amino acid targeting sequence from the Cox4 protein, while GEVO2072 has a 31 amino acid targeting sequence from the Cox4 protein. The other difference between the two strains is that GEVO2062 expresses the Aro10 protein for its KIVD enzyme, while GEVO2072 expresses the L. lactis KIVD protein. GEVO2072 consumed 16% more glucose and produced 27% more ethanol than GEVO2062 (FIG. 9). The isobutanol titers of the mitochondrial strains were 2.4 g/L and 2.1 g/L for GEVO2072 and GEVO2062 respectively at 61 h (FIG. 9).

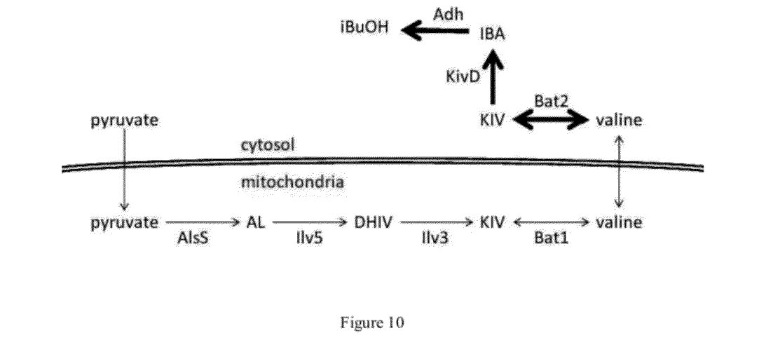

In contrast, the strain with cytosolically expressed isobutanol pathway GEVO1820 showed lower rate, titer and yield compared to the mitochondrial strains (Table 4). GEVO1820 reached 20% of the titer and productivity and 30% of the yield compared to GEVO2062. In the first 23 h of the fermentation GEVO1820 reached about 50% of the productivity and yield compared to GEVO2062 but after the 23 h time point isobutanol production was very low (FIG. 10).

TABLE 4
Productivity, Titer, and Yield for GEVO1820,
GEVO2062 and GEVO2072.

| | Specific | | | | | |
|---|---|---|---|---|---|---|
| | Productivity | | Titer | | Yield | |
| Strains | [g/L/h/OD] | ± | [g/L] | ± | % | ± |
| Gevo2072 | 0.0012 | 3.8E−05 | 2.4 | 0.1 | 2 | 0.1 |
| Gevo2062 | 0.0013 | 7.2E−05 | 2.1 | 0.1 | 2 | 0.1 |
| Gevo1820 | 0.0003 | 3.6E−05 | 0.4 | 0.03 | 0.6 | 0.06 |

### Example 33

### Determination of Enzymes Critical to Isobutanol Production

The goal of this example was to determine which pathway enzymes in GEVO2072 are critical for isobutanol production. Strains containing only two of the three pathway genes expressed in GEVO2072 were constructed by targeted directed integration of the genes to the PDC1 locus of the parent strain of GEVO2072 which is the wild-type strain GEVO1186. Strains GEVO2129 and GEVO2130 contain mitochondrially-targeted ALS and mitochondrially-targeted KIVD. The strains GEVO2127 and GEVO2128 contain a mitochondrially-targeted ALS and a cytosolically expressed ADH7. Strains GEVO2131 and GEVO2132 contain a mitochondrially-targeted KIVD protein with a cytosolically expressed ADH7. The performance of each strain in fermentation was compared to the performance of GEVO2072 to determine whether the absent enzyme is required for mitochondrial isobutanol production.

GEVO2072 (mitochondrially-targeted ALS and KIVD with a cytosolically expressed ADH) produced approximately 1.3 g/L of isobutanol by 55 hours. GEVO2127 and GEVO2128, containing the mitochondrially targeted ALS protein and the cytosolically expressed ADH like GEVO2072 but lacking mitochondrially targeted KIVD, still produced a similar isobutanol titer to GEVO2072 (Table 5).

TABLE 5
Productivity, titer, and yield at 55 hours for strains tested in the
first fermentation. GEVO2072 was run in triplicate, all other
strains were run in single fermentations. GEVO1186 is the
parent of all strains in this fermentation and serves as the
negative control.

| | Specific Productivity | | Titer | | Yield | |
|---|---|---|---|---|---|---|
| samples 55 h | [g/L/h/OD] | ± | [g/L] | ± | % | ± |
| GEVO1186 | 0.0003 | | 0.3 | | 0.6 | |
| GEVO2072 | 0.001 | 7.2E−06 | 1.3 | 0.03 | 2.3 | 0.1 |
| GEVO2127 | 0.001 | | 1.3 | | 2.2 | |
| GEVO2128 | 0.001 | | 1.2 | | 2.2 | |
| GEVO2130 | 0.0007 | | 0.8 | | 1.9 | |
| GEVO2131 | 0.0003 | | 0.3 | | 0.7 | |
| GEVO2132 | 0.0004 | | 0.5 | | 0.9 | |

Strains GEVO2129 and GEVO2130, containing mitochondrially targeted ALS and KIVD like GEVO2072, but lacking S cerevisiae ADH7 produced slightly less isobutanol than GEVO2072 at 55 hours, but by 72 hours, titers were similar to GEVO2072 (Table 6). However, all strains observed that lacked the mitochondrially targeted ALS protein (GEVO2131 and 2132) performed similar to the parental strain without the pathway and only made isobutanol at background levels (Table 5 and Table 6).

TABLE 6
Productivity, titer, and yield at 72 hours for strains tested in the second
fermentation. All strains were run in single fermentations. GEVO1186 is
the parent of all strains in this fermentation and
serves as the negative control.

| | Specific | | |
|---|---|---|---|
| | Productivity | Titer | Yield |
| samples 72 h | [g/L/h/OD] | [g/L] | [g/g] |
| GEVO1186a | 0.0002 | 0.2 | 0.2 |
| GEVO1186b | 0.0001 | 0.1 | 0.1 |
| GEVO2072a | 0.0006 | 1.1 | 1.1 |
| GEVO2072b | 0.0004 | 0.8 | 0.8 |
| GEVO2129 | 0.0004 | 0.7 | 0.7 |
| GEVO2130 | 0.0005 | 0.9 | 0.8 |

These observations suggest that ALS targeted to the mitochondrial compartment is critical to the mitochondrial isobutanol pathway, and in its absence, isobutanol cannot be produced above background concentrations. In short, of the three isobutanol pathway genes in GEVO2072, only mitochondrially-targeted ALS is required for isobutanol production. Because KIVD is not required for isobutanol production, an alternate enzyme is likely responsible for the conversion of α-keto-isovalerate to isobutyraldehyde. Likewise, because ADH7 is not required for isobutanol production, it is likely that endogenous ADH is responsible for the conversion of isobutyraldehyde to isobutanol.

### Example 34

### Introduction of BAT1 and BAT2 into GEVO2072 Leads to Increased and Sustained Isobutanol Production

An intermediate in both the valine and engineered isobutanol pathways is ketoisovalerate (KIV). The increase in isobutanol production observed upon the targeting of the B. subtilis BsalsS encoded acetolactate synthase to mitochondria, and the subsequent finding that this is the only enzyme required for the observed increase in isobutanol production, led to the proposed pathway in FIG. 10. Briefly, the pathway utilizes the exogenous acetolactate synthase activity encoded by B. subtilis BsalsS to generate acetolactate, which is subsequently utilized by the native Ilv5 (KARI), Ilv3 (DHAD), and Bat1 enzymes to produce valine. Valine crosses the inner and outer mitochondrial membranes into the cytosol, where it is subsequently converted by Bat2p into KIV. KIV is used as substrate by native KIVDs (including Pdc1 or Pdc5) for production of isobutyraldehyde, which is converted to isobutanol by either a native alcohol dehydrogenase or the overexpressed dehydrogenase encoded by S. cerevisiae ADH7.

To test the model above, the S. cerevisiae BAT2 was overexpressed in GEVO2072, a PDC-positive strain described above that contains a mitochondrially targeted acetolactate synthase from B. subtilis (alsS), a L. lactis 2-keto-acid decarboxylase (LlkivD), and a cytosolic S. cerevisiae alcohol dehydrogenase (ADH7). To overexpress BAT2, GEVO2072 was transformed with pGV1999 (SEQ ID NO: 93), a high-copy HIS3 marked yeast plasmid for production of the S. cerevisiae cytosolic Bat2p. Transformation of GEVO2072 with pGV1999 resulted in an initial increase in isobutanol production. Overexpression of the S. cerevisiae BAT2 in GEVO2072 led to a 60-70% increase in volumetric productivity (0.025±0.004 g/L/h), specific productivity (0.0027±0.004 g/L/h/OD), and yield (2.1±0.3%) compared to GEVO2072 (0.015 g/L/h, 0.0016 g/L/h/OD, and 1.3%) during the growth phase of the fermentation (Table 7). However, once in the stationary phase, the productivity in GEVO2072 with overexpressed S. cerevisiae BAT2 slowed, and the final isobutanol titer was similar to GEVO2072 at 71 hours (approximately 1 g/L).

TABLE 7
Productivity and Yields of GEVO2072 with pGV1999.

| | | |
|---|---|---|
| Vol. | Specific | |
| productivity | productivity | |
| (g/L/h) | (g/L/h/OD) | yield |

6-24 h

| | | | |
|---|---|---|---|
| pGV1999-GEVO2072 | 0.025 ± 0.004 | 0.0027 ± 0.0004 | 2.1 ± 0.3% |
| GEVO2072 | 0.015 | 0.0016 | 1.3% |

24-41 h

| | | | |
|---|---|---|---|
| pGV1999-GEVO2072 | 0.018 ± 0.006 | 0.0019 ± 0.0006 | 2.9 ± 0.9% |
| GEVO2072 | 0.015 | 0.0015 | 2.6% |

41-71 h

| | | | |
|---|---|---|---|
| pGV1999-GEVO2072 | 0.006 ± 0.003 | 0.0005 ± 0.0003 | 1.7 ± 0.7% |
| GEVO2072 | 0.014 | 0.0017 | 4.6% |

These data demonstrated that overexpression of S. cerevisiae BAT2 improves isobutanol production during log phase, suggesting that Bat2p is involved in isobutanol production in GEVO2072, and its lower expression during log phase leads to a lower rate. Because S. cerevisiae BAT1 is repressed in the stationary phase, the observed slower rate of production between 24 and 71 hours in the GEVO2072 strains with S. cerevisiae BAT2 overexpression could be the result of a reduced Bat1p activity upstream of Bat2p.

With this in mind, it was hypothesized that a reduction in S. cerevisiae BAT1 transcription as the cells entered stationary phase (Eden et al. 1996 The Journal of Biological Chemistry 271(34):20242-5) caused the observed reduction in the rate of isobutanol production in the GEVO2072 cells carrying the overexpression vector with the S. cerevisiae BAT2. To test this hypothesis, the S. cerevisiae BAT1 was cloned into a similar high-copy plasmid as the plasmid carrying the S. cerevisiae BAT2, and both plasmids were transformed into GEVO2072 and its parent strain, GEVO1186 (Table 1). In doing so, the inventors aimed to determine if overexpression of Sc_BAT1 and Sc_BAT2 from constitutive promoters on high-copy plasmids (2μ) would lead to increased and sustained isobutanol production.

To accomplish this, GEVO2072 was transformed with the S. cerevisiae BAT1 and BAT2 overexpression vectors pGV1999 and pGV2212 respectively to generate GEVO2878-GEVO2880 (Table 1). GEVO2878-GEVO2880 produced 1.2±0.1 g/L of isobutanol under low-aeration conditions in Yeast Nitrogen Base Dextrose (YNBD) medium with necessary auxotrophic supplements after 86 hours, which was 130% higher than GEVO2072 with empty vectors. Moreover, GEVO2878-GEVO2880 sustained a specific productivity of 0.005-0.006 g/L/OD/h for 86 hours, and produced isobutanol at a yield of 3.1%.

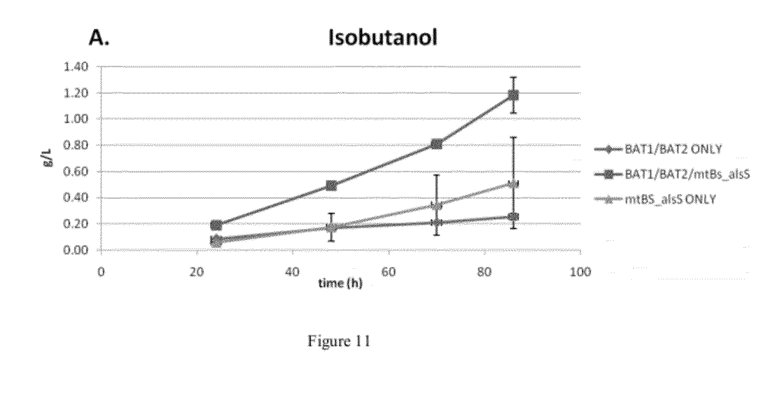

This example shows that overexpression of the S. cerevisiae BAT1 and BAT2 genes supports production of isobutanol at levels greater than double that observed with GEVO2072 after 90 hours (FIG. 11).

### Example 35

### Integration of pGV1875 into GEVO1947 to Create GEVO2087 and GEVO2088

The goal of this example was to randomly integrate the mitochondrially targeted isobutanol pathway, encoded by genes contained within pGV1875, into the genome of K marxianus. To this end, pGV1875 (SEQ ID NO: 92) was integrated into GEVO1947, and several transformants were screened for their ability to produce isobutanol in a shake flask fermentation experiment. Two transformants that produced ≧1.0 g/L isobutanol in 24 hours were named GEVO2087 and GEVO2088.

The K. marxianus strains GEVO2087 and GEVO2088 (Table 1) were constructed by transformation of GEVO1947 with pGV1875 that had been linearized with the restriction enzyme PvuI. The transformation was plated on SC-URA plates to select for integrants. Integrants were verified by colony PCR.

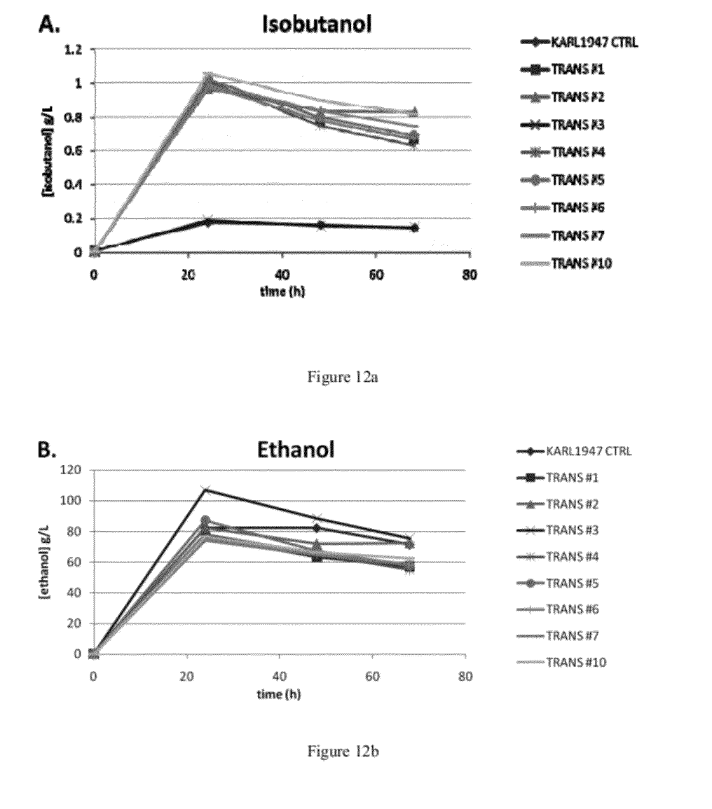

Seven of the eight transformants (#1, 2, 4-7, and 10), including GEVO2087 (Transformant #10) and GEVO2088 (Transformant #7) produced approximately 1 g/L isobutanol after 24 hours of incubation (FIG. 12a). Transformant #3 and GEVO1947 without the pathway produced less than 0.2 g/L isobutanol after 24 hours (FIG. 12a). All of the strains produced high levels of ethanol (>74 g/L), (FIG. 12b).

Table 8 summarizes the productivity, titers, and yields of isobutanol produced by the GEVO1947 transformants. All of the transformants, except #3, produced at least 1 g/L isobutanol with specific productivities of ˜0.001 g/L/h/OD isobutanol. Transformant #10 (GEVO2087) had the greatest values for productivities and titer compared to the other transformants, as well as the greatest yield (1.6%).

TABLE 8
Productivity, Titer, and Yield at 24 hrs for K. marxianus strains

| | Specific | Isobutanol | |
|---|---|---|---|
| | Productivity | Titer | Yield |
| | [g/L/h/OD] | [g/L] | % |
| GEVO1947 | 0.0001 | 0.2 | 0.2% |
| TRANS #1 | 0.0007 | 1 | 1.2% |
| TRANS #2 | 0.0014 | 1 | 1.1% |
| TRANS #3 | 0.0001 | 0.2 | 0.2% |
| TRANS #4 | 0.0007 | 1 | 1.4% |
| TRANS #5 | 0.0007 | 1 | 1.3% |
| TRANS #6 | 0.0007 | 1 | 1.3% |
| TRANS #7 | 0.0009 | 1 | 1.2% |
| (GEVO2088) | | | |
| TRANS #10 | 0.0010 | 1.1 | 1.6% |
| (GEVO2087) | | | |

This example demonstrates that GEVO1947 strains transformed with pGV1875 produced up to 1.1 g/L isobutanol in 24 hours. GEVO2087 (transformant #10) produced 1.1 g/L isobutanol in 24 hours, with a volumetric productivity of 0.044 g/L/h, a specific activity of 0.001 g L/h/OD, and a yield of 1.6%.

### Example 36

### Comparison of GEVO2087 and GEVO2072 Isobutanol Production

The goal of this example was to compare the isobutanol production in GEVO2072 and GEVO2087 in the same shake flask experiment. Aerobic batch cultivations were performed at 30° C. in 250 mL flasks in an orbital shaker at 250 rpm. Yeast cells were grown in YPD medium to mid- to late-log phase. Cells were collected and resuspended in 50 mL of fresh YPD (50 g-glucose L⁻¹) to a final OD₆₀₀ of 0.5-0.8 to initiate fermentations. Samples (2 mL) were taken at 0, 6, 24, 48, and 66.5 hours and cells were removed by centrifugation at ≧14000×g for 10 min in a microcentrifuge. Glucose concentration was measured at each time point on the YSI 2700 Select (YSI Life Sciences, Yellow Springs, Ohio, USA) according to manufacturer's protocols. After 6 hours, 9 mL of 376 g/L glucose was added to each fermentation cultures. After 24 hours, 13.5 ml of 376 g/L glucose was added. After 48 hours, 14 ml of 365 g/L glucose was added. The supernatants from the samples were collected and kept at 4° C. until analysis by Gas Chromatography and/or High Performance Liquid Chromatography as described above.

The fermentations were initiated with 50 g/L glucose (t=0), and glucose concentrations were monitored throughout the 66.5 hour fermentation experiment using the YSI (data not shown). After 6 hours, the cultures had consumed approximately 25 g/L glucose. Glucose was fed to the cultures at this time point, as well as at 24 and 48 hours. All strains tested consumed glucose at roughly the same rate up to the 48 hour time point. However, after 48 hours, the S. cerevisiae strains continued to consume glucose at a similar rate as previous timepoints (based upon similar slope between 24 and 48 hour measurements and between 48 and 66.5 hour measurements), whereas the K. marxianus strains consumed at a slower rate.

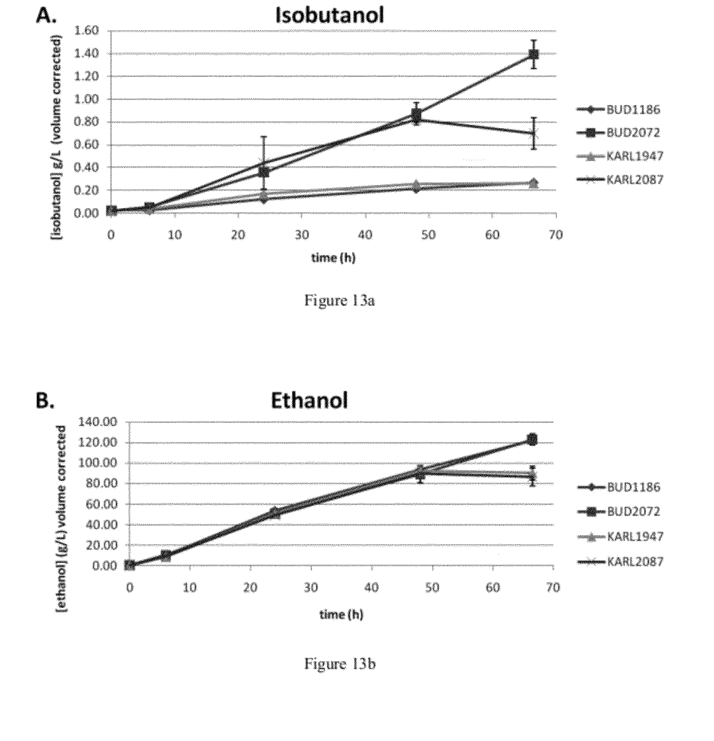

The concentrations of isobutanol and ethanol for each culture after 0, 6, 24, 48, and 66.5 hours of incubation were averaged for each strain, and the results are summarized in the charts in FIGS. 13a and 13b. After 48 hours, GEVO2087 and GEVO2072 both produced approximately 0.8 g/L isobutanol, compared to 0.2 g/L in the negative control strains, GEVO1947 and GEVO1186 (FIG. 13a). GEVO2087 did not produce anymore isobutanol after 48 hours, whereas GEVO2072 continued to produce isobutanol at a similar rate, reaching a titer of ˜1.4 g/L. All four strains produced ˜90 g/L ethanol in 48 hours (FIG. 13b). The K. marxianus strains failed to produce more ethanol after 48 hours, consistent with the slowdown in growth and glucose consumption after 48 hours.

Table 9 summarizes the productivity, titers, and yields of isobutanol produced by GEVO1186, GEVO2072, GEVO1947, and GEVO2087 after 48 and 66.5 hours. After 48 hours, the pathway carrying strains, GEVO2072 and GEVO2087, exhibited similar productivities (0.018 g/L/h and 0.0006 g/L/h/OD) and yields (˜1.1%). These values were generally 2-4 times greater than their counterparts that lacked the pathway, GEVO1186 and GEVO1947. After 66.5 hours, the productivities, titer, and yield values of GEVO2087 were lower than at the 48 hour time point, whereas GEVO2072 had the highest values observed during this experiment (specific productivity=0.0006 g/L/h/OD, titer=1.3 g/L, and yield=1.3%).

TABLE 9
Productivity, Titer, and Yield at 48 h and 66.5 h

| | | |
|---|---|---|
| Specific | Isobutanol | |
| Productivity | Titer | Yield |
| [g/L/h/OD] | [g/L] | % |

48 hours

| | | | |
|---|---|---|---|
| GEVO1186 | 0.0002 ± 0.0000 | 0.2 ± 0.02 | 0.2 ± 0.02% |
| GEVO2072 | 0.0006 ± 0.0001 | 0.8 ± 0.10 | 1.1 ± 0.12% |
| GEVO1947 | 0.0003 ± 0.0000 | 0.2 ± 0.00 | 0.3 ± 0.00% |
| GEVO2087 | 0.0006 ± 0.0000 | 0.8 ± 0.03 | 1 ± 0.03% |

66.5 hours

| | | | |
|---|---|---|---|
| GEVO1186 | 0.0002 ± 0.0000 | 0.2 ± 0.02 | 0.2 ± 0.02% |
| GEVO2072 | 0.0006 ± 0.0000 | 1.3 ± 0.12 | 1.3 ± 0.11% |
| GEVO1947 | 0.0002 ± 0.0000 | 0.2 ± 0.02 | 0.3 ± 0.03% |
| GEVO2087 | 0.0004 ± 0.0001 | 0.6 ± 0.14 | 0.8 ± 0.18% |

### Example 37

### Introduction of Mitochondrially-Targeted Isobutanol Pathway into Strain Lacking PDC Activity

A Pdc-K. marxianus strain (GEVO1969) was transformed with a mitochondrially-targeted isobutanol pathway encoded by pGV1875 (SEQ ID NO: 92) to construct GEVO2276 and GEVO2277 (Table 1). Shake flask fermentations were performed and four strains were analyzed (GEVO2087, GEVO1969, GEVO2276, and GEVO2277).

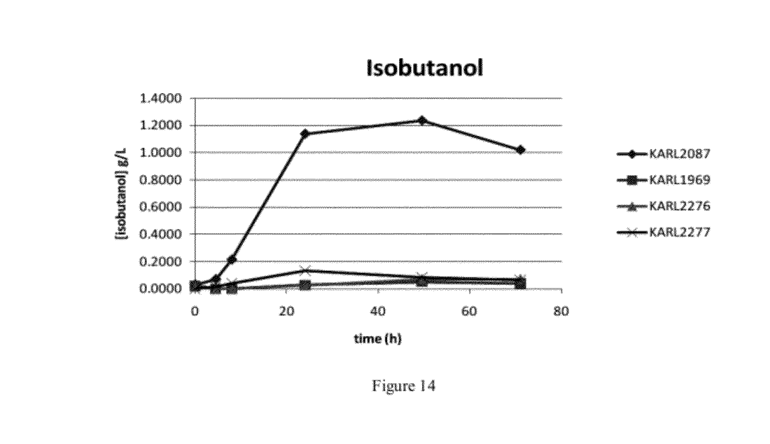

As shown in FIG. 14, GEVO2277, which contains the entire mitochondrially-targeted pathway (PATHWAY+), but lacks PDC activity (Pdc-) produced approximately 10% the amount of isobutanol as GEVO2087 (PATHWAY+ PDC+) after 24 hours incubation. GEVO2277 produced approximately 4 times more isobutanol after 24 hrs than did GEVO1969 or GEVO2276, neither of which contained B. subtilis BsalsS transcript.

As discussed above, GEVO2277 produced approximately 10% the isobutanol compared to GEVO2087 after 24 hours incubation, suggesting that PDC activity is required for production of isobutanol (Titer column in Table 10). However, the specific productivity of GEVO2277 was closer to one-third that of GEVO2087, and the yield of GEVO2277 was approximately half of GEVO2087. The difference in relative yields and specific productivities compared to the relative titers reflects the slower growth and glucose consumption of the Pdc-minus GEVO2277.

TABLE 10
Summary of Isobutanol Production after 24 hours Incubation

| | | specific | |
|---|---|---|---|
| | Titer | productivity | |
| | (g/L) | (g/L/h/OD₆₀₀) | yield |
| GEVO2087 | 1.1 | 0.0016 | 2.1% |
| GEVO1969 | 0.03 | 0.0001 | 0.3% |
| GEVO2276 | 0.03 | 0.0001 | 0.4% |
| GEVO2277 | 0.13 | 0.0005 | 1.1% |

While GEVO2087 produced more isobutanol than the other three metabolites combines, GEVO2277 produced mostly acetoin, as well as greater levels of diacetyl and pyruvate. Generally, the distribution suggests that the pathway is blocked after the conversion of pyruvate to acetolactate, which is catalyzed by the enzyme encoded by B. subtilis BsalsS. Because both GEVO2087 and GEVO2277 are using the native KARI and DHAD enzymes in the mitochondria, neither of the enzyme are likely the bottleneck in the pathway. However, because GEVO2087 contains PDC activity and GEVO2277 does not, this appears to be the activity that is required for production of isobutanol. This suggests that KIVD activity is limiting, and PDC activity is required for the majority of this activity.

### Example 38

### Introduction of B. subtilis BsalsS with MTS and L. lactis LlkivD2 Lacking MTS into Strain Lacking PDC Activity

In order to eliminate ethanol production and to produce isobutanol, the mitochondrially-targeted pathway was introduced into a Pdc-minus K. marxianus strain (GEVO1969). The resulting strain, GEVO2277, produced 0.13 g/L isobutanol, which was 4-times higher than background. However, GEVO2277 only produced isobutanol at a yield of 1.1%, while its PDC+ counterpart, GEVO2087, produced isobutanol at a yield of 2.1% (see Table 11).

One possible reason for the reduction in isobutanol yield in the Pdc-minus GEVO2277 strain is the loss of ketoisovalerate decarboxylase (KIVD) activity contributed by PDC. To test this hypothesis, the Pdc-minus GEVO1969 strain was transformed with a plasmid that contained B. subtilis BsalsS with a mitochondrial targeting sequence and L. lactis LlkivD2 lacking a mitochondrial targeting sequence (pGV1990) (SEQ ID NO: 94), or a plasmid that only contained B. subtilis BsalsS with a mitochondrial targeting sequence (pGV2015) (SEQ ID NO: 95).

As shown in Table 11, pGV1990-GEVO1969 #5 (GEVO 2347) produced approximately 0.3 g/L isobutanol in 24 hours, which was the highest measured isobutanol titer in this experiment, while producing only 0.001 g/L ethanol. pGV2015-GEVO1969 #2 (GEVO2348) produced the second highest isobutanol, approximately 0.15 g/L. pGV1990-GEVO1969 #1 and #4 produced similar isobutanol titers (−0.07-0.09 g/L) as the positive control strain, GEVO2277, which was approximately 3-4× higher than the negative control strain, GEVO1969. pGV2015-GEVO1969 #3 produced less isobutanol than the negative control strain.

TABLE 11
Summary of Isobutanol Production after 24 Hrs Incubation

| | | | Specific | ethanol |
|---|---|---|---|---|
| | isobutanol titer | | productivity | titer |
| | (g/L) | yield | (g/L/h/OD) | (g/L) |
| pGV1990-1 | 0.07 | 0.8% | 0.0003 | 0.001 |
| pGV1990-4 | 0.08 | 1.0% | 0.0003 | 0.000 |
| (GEVO2346) | | | | |
| pGV1990-5 | 0.3 | 3.3% | 0.0013 | 0.001 |
| (GEVO2347) | | | | |
| pGV2015-2 | 0.14 | 1.4% | 0.0004 | 0.005 |
| (GEVO2348) | | | | |
| pGV2015-3 | 0.01 | 0.2% | 0.0001 | 0.019 |
| GEVO1969 | 0.02 | 0.5% | 0.0001 | 0.001 |
| GEVO2277 | 0.08 | 1.0% | 0.0004 | 0.000 |

### Example 39

### Determination of KIVD Activity

Based upon the qRT-PCR and shake flask fermentation data, three of the transformants were saved and renamed with the following designations: GEVO2346, GEVO2347, and GEVO2348. To determine the ketoisovalerate decarboxylase activity in these three strains, along with several other control strains, KIVD activity assays were performed on whole cell lysates.

Cells from a saturated 3 mL YPE cultures (incubated overnight at 30° C., 250 RPM) were used to inoculate 25 mL YPD cultures at OD₆₀₀ of 0.3-0.4 in 125 mL metal capped, baffled flasks. The cultures were incubated at 30° C., 250 RPM for 5-6 hours. The cells were collected by centrifugation (5 minutes at 1600×g), and the mass of the pellet was weighed after removal of the supernatant. KIVD Assay buffer (see below for recipe), containing 1 Roche Protease Inhibitor tablet per 5 mL buffer, was added to each pellet to create a 20% (w/v) cell suspension. Cell lysates were prepared by bead beating and the final protein concentration was determined by Bradford assay. The quality of the isolated proteins was verified with a Coomassie Blue Stained gel.

Ketoisovalerate decarboxylase activity was assessed as follows. A reaction buffer was prepared at a final concentration of 0.05 M NaHPO₄*H₂O, 5 mM MgCl₂*8H₂O, and 1.5 mM Thiamin pyrophosphate chloride. The reaction substrate, α-keto-isovalerate (3-methyl-2-oxobutanoic acid, Acros Organics), was added where appropriate at 30 mM. Lysates were diluted in reaction buffer at a final protein concentration of 0.1 μg/μL. To 1.5 mL tubes, 50 μL of lysate (5 μg of protein) was mixed with 200 μL of reaction buffer with or without substrate. The reactions were incubated at 37° C. for 20 minutes, and the reactions were immediately filtered through a 2 μm filter plate. The filtered samples were diluted 1:10 in water, and 100 μL of the 1:10 dilution was mixed with 100 μL of derivatization reagent in a 0.2 ml thin-wall PCR tubes. Derivatization reagent was prepared by mixing 4 ml of 2,4-Dinitrophenyl Hydrazine (DNPH) in 15 mM in HPLC-grade Acetonitrile with 1 ml 50 mM Citric Acid Buffer, pH 3. The samples were incubated at 70° C. for 30 minutes. The samples were analyzed by HPLC.

Analysis of ketoisovalerate and isobutyraldehyde, derivatized with DNPH, was performed on a HP-1100 High Performance Liquid Chromatography system equipped with a Hewlett Packard 1200 HPLC stack column (Agilent Eclipse XDB-18, 150×4.0 mm; 5 μm particles [P/N #993967-902] and C18 Guard cartridge). Ketoisovalerate and isobutyraldehyde were detected using an HP-1100 UV detector (360 nm). The column temperature was 50° C. This method was Isocratic with 60% acetonitrile, 1% H₃PO₄ in Milli-Q water. Flow was set at 1.0 mL/min. Injection size was 10 μL and the run time was 10 minutes. KIVD activity is summarized in Table 12. As shown in Table 12, GEVO2347 contained approximately 33% more KIVD activity than did the negative, Pdc-minus control (GEVO1969), which was determined to be a statistically significant difference (t-test, p=0.045). The increase in KIVD activity in GEVO2347 correlates with the increase in isobutanol production, suggesting that cytosolic KIVD activity was limiting for isobutanol production in the Pdc-minus strains.

TABLE 12
KIVD Activity.

| | Genotype | | | | Transformant | Specific KIVD |
|---|---|---|---|---|---|---|
| Strain | PDC | ALS | KIVD | ADH | Designation | activity (U/mg) |
| GEVO1947 | + | − | − | − | N/A | 0.166 ± 0.029 |
| GEVO1969 | − | − | − | − | N/A | 0.061 ± 0.005 |
| GEVO2087 | + | mitochondria | mitochondria | cytosol | N/A | 0.216 ± 0.025 |
| GEVO2277 | − | mitochondria | mitochondria | cytosol | N/A | 0.058 ± 0.003 |
| GEVO2346 | − | mitochondria | cytosol | − | pGV1990 #4 | 0.059 ± 0.005 |
| GEVO2347 | − | mitochondria | cytosol | − | pGV1990 #5 | 0.080 ± 0.010 |
| GEVO2348 | − | mitochondria | − | − | pGV2015 #2 | 0.047 ± 0.007* |

*Only 2 replicates.

### Example 40

### High Total Titer with Mitochondrially Targeted ALS and KIVD in K. marxianus

GEVO2087 is a modified yeast biocatalyst that contains genes within the chromosome of the biocatalyst which encode a pathway of enzymes that convert pyruvate into isobutanol. When the biocatalyst GEVO2087 was contacted with glucose in a medium suitable for growth of the biocatalyst, at about 30° C., the biocatalyst produced isobutanol from the glucose. An overnight starter culture was started in a 250 mL Erlenmeyer flask with GEVO2087 cells from a freezer stock with a 40 mL volume of YPD medium consisting of 100 g/L glucose, 10 g/L yeast extract, 20 g/L peptone and at a culture OD600 of 0.05 to 0.1. The starter culture was grown for approximately 14 hrs in a 30° C. shaker at 250 rpm. Some of the starter culture was then transferred to a 2000 mL DasGip fermenter vessel containing about 1500 mL of YPD medium containing 150 g/L glucose initially to achieve an initial culture OD600 of about 0.1. The vessel was attached to a computer control system to monitor and control pH at 6.5 through addition of base, temperature at about 30° C., dissolved oxygen, and agitation. Initially, during the cell growth phase, the vessel was agitated with a fixed agitation of 600 rpm using a 10 sL/h air sparge until the OD600 was about 31. Cell growth continued for approximately 14 hrs, after which time, the agitation was decreased to 400 rpm with 10 sL/h airflow. The dissolved oxygen was approximately zero throughout this experiment. Continuous measurement of the fermentor vessel off-gas by GC-MS analysis was performed for oxygen, isobutanol, ethanol, and carbon dioxide throughout the experiment. Samples were aseptically removed from the fermenter vessel throughout the experiment and used to measure OD600, glucose concentration, and isobutanol concentration in the broth. At about 196 h into the experiment, the fermenter whole broth was removed from the fermenter, cells were separated from the broth using centrifugation at about 20° C. an 4000×g in 500 mL centrifuge bottles. The cell pellets were resuspended in fresh YPD medium that contained 150 g/L glucose and returned to the fermenter. A glucose feed of about 600 g/L glucose in DI water was used intermittently during the production phase of the experiment at time points greater than 14 h to maintain glucose concentration in the fermenter of about 100 g/L or above.

Isobutanol was recovered from this fermentation using methods that are described in US Patent Publication US 20090171129, which is hereby incorporated by reference in its entirety.

The distillate recovered in the experiment was strongly enriched for isobutanol. Distillate samples were analyzed by GC for isobutanol concentration. Isobutanol production reached a maximum at around 288 hrs with a total titer of about 21.6 g/L.

### Example 41

### Construction and Fermentation of Isobutanol Pathway Strains Derived from S. cerevisiae Using Different Genes for ALS and KIVD and Using Different Mitochondrial Targeting Sequences for Bs_AlsS

Two independent shake flask fermentations were performed. The first fermentation was performed in order to screen several strains with different mitochondrially targeted isobutanol pathways for isobutanol production, and to compare these strains to GEVO2072. All of the strains, including GEVO2072, have the pathway integrated at the PDC1 locus, the difference between the strains is the combinations of enzymes and mitochondrial targeting sequences used. For each new strain two clones were tested, each in a single shake flask.

The second fermentation was performed with GEVO2072 and GEVO2119, both of which were run in triplicate.

In the first fermentation, of the strains tested, two performed equivalent to GEVO2072 in isobutanol titer. These two strains, GEVO2120 and GEVO2121 were both constructed by integration of pGV1877 into GEVO1186, and they differ from GEVO2072 in that they have ARO10 in place of LlkivD. GEVO2122 thru GEVO2126 all produced approximately the same isobutanol titer of 0.5 g/L at 55 hours. GEVO2122 and GEVO2123 were both constructed by integration of pGV1878 into GEVO1186, and this pathway consists of ILV2, kivD, and ADH7. GEVO2124 and GEVO2125 were both constructed by integration of pGV1879 into GEVO1186, and this pathway consists of ILV2, ARO10, and ADH7. GEVO2126 was constructed by integration of pGV1892 into GEVO1186, and this pathway consists of ILV2, LlkivD, and ILV6*. ILV6* is a mutant of ILV6 that is not repressed by valine. The performance of these strains suggests that ALS is critical to the mitochondrial isobutanol pathway in GEVO2072, GEVO2120, and GEVO2121. All strains consumed approximately the same amount of glucose at all time points.

In the second fermentation, GEVO2072 again performed as expected and produced about 1 g/L of isobutanol by 55 hours. At 55 hours, GEVO2119 made approximately the same amount of isobutanol as GEVO2072, producing 0.94 g/L, and this value is within the error range of GEVO2072. Both strains had similar values for specific productivity and yield. Both strains consumed similar amounts of glucose and reached similar cell densities.

### Example 42

### Integration of a Mitochondrial Isobutanol Pathway into a PDC Minus S. cerevisiae Strain

To generate a Pdc-S. cerevisiae strain that expresses the mitochondrial isobutanol pathway, the Pdc-strain GEVO1584 was transformed with pGV1874 that was linearized using PvuI. The resulting Ura+ transformants, GEVO2166 and GEV2167, express mitochondrially-targeted ALS and KIVD proteins and a cytosolically expressed ADH7 protein.

GEVO1584 and the two transformants were grown in YPE to generate biomass before inoculation of YPD cultures at an OD of 1 to perform shake flask fermentations. None of the candidates produced isobutanol above background. None of the transformants produced ethanol above the background level produced by GEVO1584.

GEVO2166 and GEVO2167 were inoculated at a similar initial OD as GEVO1584 and grew approximately one generation after the transition to glucose, whereas GEVO1584 grew approximately 2 generations. None of the strains consumed all of the glucose in the initial culture (50 g/L). The strains' glucose consumption tracked with growth (e.g. GEVO1584 consumed approximately twice as much glucose and grew to an OD that was double compared to GEVO2166).

S. cerevisiae PDC minus strain GEVO1584 shows 6 times less KIVD in vitro activity than a K. marxianus Pdc minus strain GEVO1969. When Ll_kivd (codon optimized for E. coli) is overexpressed in these strains KIVD activity is 20-25 times increased compared to the parent strains (Table 13). The low KIVD activity in GEVO1584 provides one explanation why the corresponding mitochondrial isobutanol pathway carrying strain GEVO2302 did not produce isobutanol over background level. Overexpression of cytosolic KIVD in this Pdc-minus pathway strain might improve its productivity as was shown for K. marxianus.

TABLE 13
Specific KIVD activity in K. marxianus and S. cerevisiae Pdc-strains.

| Strain | KIVD over expression | Spec activity [U/mg] |
|---|---|---|
| GEVO1584 | No | 0.08 |
| GEVO2302 | Yes | 2.3 |
| GEVO1969 | No | 0.49 |
| GEVO2542 | Yes | 12.5 |

### Example 43

### Fermentation of a Mitochondrial Isobutanol Pathway Carrying Strain in Batch Fermenters

A preculture of 80 mL YPD with 50 g/L glucose was inoculated from a single colony and incubated at 250 rpm, 30° C. for 24 hours. The preculture was transferred to 2 fernbach flasks with 1000 mL YPD with 50 g/L glucose and incubated for 14 hours at 30° C. The starting OD600 of the fernbach flasks was 0.05 and the cultures were harvested at an OD600 of 4.4. The cultures were centrifuged and resuspended as a concentrate. Dasgip 300 mL fermenter vessels with 200 mL YPD+150 g/L glucose were inoculated with the concentrate to a starting OD600 of 5-6. The fermenters were run at a temperature of 30° C. The pH was controlled at 5.5 with 2N KOH and the air flow rate was 2.5 sL/h. The starting agitation was 400 rpm and the agitation was used to control the dissolved oxygen at 5%. One of the fermentation vessels (Vessel 5) was switched to anaerobic by sparging with nitrogen at 25 hours. The results of the fermentations are summarized in Table 14.

TABLE 14
Titer, yield and productivity reached in fermentations of GEVO2062.

| | | Vessel | Vessel | Vessel | Vessel |
|---|---|---|---|---|---|
| | | 5 | 6 | 7 | 8 |
| Max Titer | g I-BuOH/L | 1.93 | 2.73 | 2.34 | 2.49 |
| Yield | % theor. I-BuOH | 1.5% | 2.3% | 1.9% | 2.0% |
| Productivity | g I-BuOH/L/hr | 0.03 | 0.04 | 0.04 | 0.04 |
| Specific | g I-BuOH/OD/L/hr | 0.0019 | 0.0032 | 0.0027 | 0.0028 |
| Productivity | | | | | |

The foregoing detailed description has been given for clearness of understanding only and no unnecessary limitations should be understood there from as modifications will be obvious to those skilled in the art.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth and as follows in the scope of the appended claims.

The disclosures, including the claims, figures and/or drawings, of each and every patent, patent application, and publication cited herein are hereby incorporated herein by reference in their entireties.

1. A recombinant eukaryotic microorganism capable of producing isobutanol from a carbon source, said recombinant eukaryotic microorganism comprising an isobutanol producing metabolic pathway, wherein said metabolic pathway comprises enzymes catalyzing the conversions (a-e):
a) pyruvate to acetolactate;
b) acetolactate to dihydroxyisovalerate;
c) dihydroxy isovalerate to ketoisovalerate;
d) ketoisovalerate to isobutyraldehyde; and
e) isobutyraldehyde to isobutanol,
and wherein at least one of the conversions (a-e) occurs in the mitochondria.

2. The recombinant eukaryotic microorganism of claim 1, wherein the at least one conversion is (a) pyruvate to acetolactate.

3. The recombinant eukaryotic microorganism of claim 1, wherein at least two of the conversions (a-e) occur in the mitochondria.

4. The recombinant eukaryotic microorganism of claim 1, wherein at least three of the conversions (a-e) occur in the mitochondria.

5. The recombinant eukaryotic microorganism of claim 1, wherein at least four of the conversions (a-e) occur in the mitochondria.

6. The recombinant eukaryotic microorganism of claim 1, wherein all five of the conversions (a-e) occur in the mitochondria.

7. A recombinant eukaryotic microorganism capable of producing isobutanol from a carbon source, said recombinant eukaryotic microorganism comprising an isobutanol producing metabolic pathway, wherein said metabolic pathway comprises at least one of the enzymes selected from (a-f):
a) acetolactate synthase (ALS);
b) ketolacid reductoisomerase (KARI);
c) dihydroxy acid dehydratase (DHAD);
d) ketoisovalerate decarboxylase (KIVD);
e) alcohol dehydrogenase (ADH); and a
f) branched chain amino acid aminotransferase,
and wherein at least one of the enzymes (a-f) is overexpressed and targeted to the mitochondria.

8. The recombinant eukaryotic microorganism of claim 7, wherein the at least one enzyme is ALS.

9. The recombinant eukaryotic microorganism of claim 7, wherein at least two of the enzymes (a-f) are overexpressed and targeted to the mitochondria.

10. The recombinant eukaryotic microorganism of claim 7, wherein at least three of the enzymes (a-f) are overexpressed and targeted to the mitochondria.

11. The recombinant eukaryotic microorganism of claim 7, wherein at least four of the enzymes (a-f) are overexpressed and targeted to the mitochondria.

12. The recombinant eukaryotic microorganism of claim 7, wherein five of the enzymes (a-f) are overexpressed and targeted to the mitochondria.

13. The recombinant eukaryotic microorganism of claim 1, wherein said recombinant eukaryotic microorganism produces isobutanol at a specific productivity of at least about 0.003 g/L/h/OD.

14. The recombinant eukaryotic microorganism of claim 1, wherein said recombinant eukaryotic microorganism produces isobutanol at a titer of at least about 2.7 g/L.

15. The recombinant eukaryotic microorganism of claim 1, wherein said recombinant eukaryotic microorganism produces isobutanol at a total titer of at least about 21 g/L.

16.-17. (canceled)

18. The recombinant eukaryotic microorganism of claim 1, wherein said recombinant eukaryotic microorganism overexpresses BAT1 or BAT2.

19.-25. (canceled)

26. The recombinant eukaryotic microorganism of claim 1, wherein said recombinant eukaryotic microorganism is engineered to overexpress one or more genes selected from PCK1, PYC1, PYC2, MDH2, DIC1 and MAE1.

27. (canceled)

28. The recombinant eukaryotic microorganism of claim 1, wherein said recombinant eukaryotic microorganism is further engineered to express a transhydrogenase.

29. The recombinant eukaryotic microorganism of claim 28, wherein said transhydrogenase is localized to the cytoplasmic membrane.

30. The recombinant eukaryotic microorganism of claim 28, wherein said transhydrogenase is localized to the mitochondrial membrane.

31. The recombinant eukaryotic microorganism of claim 28, wherein said transhydrogenase is localized to the cytoplasmic membrane and the mitochondrial membrane.

32. The recombinant eukaryotic microorganism of claim 28, wherein said transhydrogenase is a mammalian transhydrogenase.

33. The recombinant eukaryotic microorganism of claim 28, wherein said transhydrogenase is a bacterial membrane bound transhydrogenase.

34. The recombinant eukaryotic microorganism of claim 28, wherein said transhydrogenase is a fungal transhydrogenase.

35. The recombinant eukaryotic microorganism of claim 34, wherein said fungal transhydrogenase is a transhydrogenase derived from Neurospora crassa.

36.-40. (canceled)

41. The recombinant eukaryotic microorganism of claim 1, wherein said recombinant eukaryotic microorganism is engineered to have reduced pyruvate decarboxylase (PDC) activity.

42.-43. (canceled)

44. The recombinant eukaryotic microorganism of claim 1, wherein said recombinant eukaryotic microorganism is engineered to have reduced glycerol-3-phosphate dehydrogenase (GPD) activity.

45.-53. (canceled)

54. The recombinant eukaryotic microorganism of claim 1, wherein said recombinant eukaryotic microorganism is a yeast recombinant microorganism.

55.-69. (canceled)

70. A method of producing isobutanol, comprising the steps of:
(a) providing a recombinant eukaryotic microorganism according to claim 1: and
(b) cultivating said recombinant eukaryotic microorganism in a culture medium containing a feedstock providing a carbon source until the isobutanol is produced.

71.-72. (canceled)
